(19) United States
(12) Patent Application Publication
(10) Pub. No.: US 2010/0113959 A1
(43) Pub. Date: May 6, 2010

(54) TRANSCRANIAL MAGNETIC STIMULATION (TMS) METHODS AND APPARATUS

(75) Inventors: Alvaro Pascual-Leone, Wayland, MA (US); Timothy Wagner, Cambridge, MA (US)

Correspondence Address: WOLF GREENFIELD & SACKS, P.C., 600 ATLANTIC AVENUE, BOSTON, MA, 02210-2206, US

(73) Assignee: BETH ISRAEL DEACONESS MEDICAL CENTER, INC., Boston, MA (US)

(21) Appl. No.: 12/224,770

(22) PCT Filed: Mar. 7, 2007
(86) PCT No.: PCT/US2007/005893

§ 371 Date: Jan. 25, 2010

Related U.S. Application Data
(60) Provisional application No. 60/779,847, filed on Mar. 7, 2006.

(51) Int. Cl. A61B 5/0478; A61N 2/02

(52) U.S. Cl. 600/544; 600/13

(57) ABSTRACT

In one aspect, a portable transcranial magnetic stimulation (TMS) device for delivering a TMS procedure is provided. The portable TMS device comprises at least one coil that, when energized, generates electromagnetic energy, a helmet adapted to fit a user's head and configured to hold the at least one coil in a predetermined position with respect to the user's head, and a port having at least one power connection coupled to the at least one coil, the at least one power connection adapted to connect the at least one coil to a power source capable of energizing the at least one coil, the port further comprising at least one data connection adapted to exchange data with at least one external component, the port being located on the helmet. In another aspect a method of positioning a coil with respect to a person's head to target a desired region of the person's brain with transcranial magnetic stimulation (TMS) is provided. The method comprises obtaining a dielectric property map of a portion of the brain, the dielectric map indicating a spatial distribution of at least one dielectric property over the portion of the brain, determining a location for the coil based, at least in part, on the dielectric property map, the location being such that when the coil is positioned at the location, magnetic energy generated by the coil is focused on the desired region, and positioning the coil at the determined location.

### RELATED APPLICATION

This application claims priority under 35 U.S.C. §119(e) to U.S. Provisional Application Ser. No. 60/779,847, entitled “TRANSCRANIAL MAGNETIC STIMULATION (TMS) METHODS AND APPARATUS,” filed on Mar. 7, 2006, which is herein incorporated by reference in its entirety.

### FIELD OF INVENTION

The present invention relates to transcranial magnetic stimulation (TMS), and more particularly, to TMS devices and methods of providing TMS therapy.

### BACKGROUND

Transcranial magnetic stimulation (TMS) provides a non-invasive procedure for generating magnetic fields to induce stimulating electric current to desired areas of the human body, typically targeted areas of the brain. In general, TMS procedures involve identifying a discrete region of the brain and focusing magnetic fields generated by one or more coils positioned proximate the head at a location that induces electric current in the identified region of the brain. The type and character of the magnetic field deposition, and the location of the targeted region of the brain typically depends on the type of therapeutic and/or diagnostic application that is to be achieved.

A growing understanding of TMS techniques has led to the development of numerous diagnostic and therapeutic applications in neurology, cognitive neuroscience, clinical neurophysiology, psychiatry, neurorehabilitation, etc. Repetitive Transcranial Magnetic Stimulation (rTMS) has been used as noninvasive treatment for a variety of medical conditions. Applications for rTMS include, but are not limited to, psychiatric disorders, such as depression, hallucinations, obsessions, and drug craving; neurologic diseases such as Parkinson's disease, dystonia, or epilepsy; rehabilitation of aphasia or of hand function after stroke; and pain syndromes, such as caused by migraine, neuropathies, low back pain, or internal visceral diseases such as chronic pancreatitis or cancer.

### SUMMARY OF THE INVENTION

Some embodiments according to the present invention include a portable transcranial magnetic stimulation (TMS) device for delivering a TMS procedure comprising at least one coil that, when energized, generates electromagnetic energy, and a helmet adapted to fit a user's head and configured to hold the at least one coil in a predetermined position with respect to the user's head.

Some embodiments according to the present invention include a portable transcranial magnetic stimulation (TMS) device for delivering a TMS procedure comprising at least one coil that, when energized, generates electromagnetic energy, a helmet adapted to fit a user's head and configured to hold the at least one coil in a predetermined position with respect to the user's head, and a port having at least one power connection coupled to the at least one coil, the at least one power connection adapted to connect the at least one coil to a power source capable of energizing the at least one coil, the port further comprising at least one data connection adapted to exchange data with at least one external component, the port being located on the helmet.

Some embodiments according to the present invention include a method of positioning a coil with respect to a person's head to target a desired region of the person's brain with transcranial magnetic stimulation (TMS). The method comprises obtaining a dielectric property map of a portion of the brain, the dielectric map indicating a spatial distribution of at least one dielectric property over the portion of the brain, determining a location for the coil based, at least in part, on the dielectric property map, the location being such that when the coil is positioned at the location, magnetic energy generated by the coil is focused on the desired region, and positioning the coil at the determined location.

### BRIEF DESCRIPTION OF THE DRAWINGS

FIGS. 1A-1E illustrate various components of a portable TMS device, in accordance with some embodiments of the present invention;

FIG. 2 illustrates a portable TMS device, in accordance with some embodiments of the present invention;

FIG. 3 illustrates a portion of a TMS device, in accordance with some embodiments of the present invention;

FIG. 4 illustrates a portable TMS device adapted to communicate over a network, in accordance with some embodiments of the present invention;

FIGS. 5A-5B illustrates a side-view and a bottom-view of a portable TMS device having a communications port, in accordance with some embodiments of the present invention;

FIG. 6 illustrates a flow chart of a method for positioning a TMS coil, in accordance with some embodiments of the present invention;

FIG. 7 illustrates a flow chart of another method for positioning a TMS coil, in accordance with some embodiments of the present invention;

FIG. 8 illustrates a portable TMS device having EEG feedback, in accordance with some embodiments of the present invention;

FIG. 9 illustrates a frameless stereotactic system for TMS positioning, in accordance with some embodiments of the present invention;

FIG. 10 illustrates plots of current density magnitudes as evaluated along parallel lines that penetrate a head model through tissues in skin, skull, CSF, gray matter, and white matter, in accordance with some embodiments of the present invention;

FIG. 11 illustrates electromagnetic current density fields revealed via a field solver with the underlying white matter architecture as revealed via DSI, in accordance with some embodiments of the present invention;

FIG. 12 illustrates that the effects of TMS on the targeted brain region depend critically on the frequency of stimulation and are different during the time of TMS application, in accordance with some embodiments of the present invention;

FIG. 13 illustrates a system for EEG-controlled TMS, in accordance with some embodiments of the present invention;

FIG. 14 illustrates a resistor model for a head system, in accordance with some embodiments of the present invention;

FIG. 15 illustrates a profile for a single pulse magnetic stimulator, in accordance with some embodiments of the present invention;

FIG. 16 illustrates non-dimensional pulse spectrum of typical TMS current pulse waveforms, in accordance with some embodiments of the present invention;

FIG. 17 illustrates a coordinate system used to formulate field components;

FIG. 18 illustrates a passive cable model, in accordance with some embodiments of the present invention;

FIG. 19 illustrates the Hodgkin and Huxley model including the voltage/time dependent sodium and potassium channels;

FIG. 20 illustrates a head model, in accordance with some embodiments of the present invention;

FIG. 21 illustrates plots of current density and current density vector components, in accordance with some embodiments of the present invention;

FIG. 22 illustrates plots showing maximum cortical current densities, in accordance with some embodiments of the present invention;

FIG. 23 illustrates plots of current densities along a line, in accordance with some embodiments of the present invention;

FIG. 24 illustrates cortical current distribution, in accordance with some embodiments of the present invention;

FIG. 25 illustrates cortical current and normal cortical current distributions, in accordance with some embodiments of the present invention;

FIG. 26 illustrates current densities along lines through maximum points in cortex, in accordance with some embodiments of the present invention;

FIGS. 27A and 27B display pre and post stroke gray matter conductance and pre and post stroke gray matter permittivity, respectively, in accordance with some embodiments of the present invention;

FIGS. 28A and 28B illustrate skin permittivity and conductivity, respectively, in accordance with some embodiments of the present invention;

FIGS. 29A and 29B illustrate white matter conductivity and white matter permittivity, respectively, in accordance with some embodiments of the present invention;

FIG. 30 illustrates a healthy head model, in accordance with some embodiments of the present invention;

FIG. 31 illustrates that the areas of least resistance in the stroke model were found along the edge of the infarction region, in accordance with some embodiments of the present invention;

FIG. 32 illustrates current density magnitudes and perturbations for various coil positions, in accordance with some embodiments of the present invention;

FIG. 33 illustrates current density magnitudes and perturbations for various coil positions, in accordance with some embodiments of the present invention;

FIG. 34 illustrates gray matter vector plots for the healthy head model in a particular coil position, in accordance with some embodiments of the present invention;

FIG. 35 plots the current density vector distribution in the CSF for the healthy head model and the stroke model with the coil in a particular coil position, in accordance with some embodiments of the present invention;

FIG. 36 illustrates gray matter vector plots for the healthy head model in a particular coil position, in accordance with some embodiments of the present invention;

FIG. 37 plots the % difference between the healthy head model and a stroke model vs. increasing coil distance from the stroke boundary, in accordance with some embodiments of the present invention;

FIGS. 38A and 38B illustrates changes in execution time and performance, respectively, for various tasks, in accordance with some embodiments of the present invention;

FIG. 39 illustrates changes in performance of the control group, in accordance with some embodiments of the present invention;

FIG. 40 illustrates the healthy head model and the model coordinate system, in accordance with some embodiments of the present invention;

FIG. 41 illustrates current density magnitudes: the current density magnitudes are plotted for the dorsal lateral prefrontal cortex (DLPC) and the motor strip (MS) coil position, in accordance with some embodiments of the present invention;

FIG. 42 illustrates graphical examples of the cortical current density behavior along the center evaluation line for the healthy head model, in accordance with some embodiments of the present invention;

FIG. 43 illustrates a healthy head model and stroke locations, in accordance with some embodiments of the present invention;

FIG. 44 illustrates cortical current densities for various electrode montages in the healthy head model, in accordance with some embodiments of the present invention;

FIG. 45 illustrates current density behavior through tissues, and more particularly, current density magnitude evaluated along an evaluation line in the healthy head model for a particular electrode montage, in accordance with some embodiments of the present invention;

FIG. 46 illustrates skin surface current density for an electrode montage, in accordance with some embodiments of the present invention;

FIG. 47 illustrates cortical current densities for various electrode montages in the healthy head model, in accordance with some embodiments of the present invention; and

FIG. 48 illustrates cortical current densities for the strokes illustrated in FIG. 43, in accordance with some embodiments of the present invention.

### DETAILED DESCRIPTION

For many applications, effective rTMS is facilitated by routine stimulation according to a prescribed schedule. For example, a particular therapeutic application may have the best results when repeated for several days (e.g., ten to twenty days) on a daily or even bi-daily regimen. Other applications may require more or less frequent stimulation and/or require a fewer or greater duration over which the stimulation is repeated. For example, the prescribed duration may vary from a couple of days, to several weeks, months or years. In some circumstances, the most effective treatment may involve an indefinite stimulation regimen. In particular, after the initial treatment (referred to as the induction phase), a patient may require (or respond more positively) by continuing with maintenance therapy for many months, and possibly indefinitely.

Conventionally, such rTMS procedures are achieved through prescribed visits from the patient to the hospital, doctor's office or laboratory where the TMS equipment is located. Such routine visits may be inconvenient and invasive to the patient's life, who may have work, family and/or other responsibilities that make following the prescribed regimen of office visits difficult, perhaps prohibitively so. Moreover, the availability of the doctor, holidays and/or other circumstances where an office visit is impossible may disrupt the proper execution of the treatment.

Applicant has appreciated that a generally portable TMS device may facilitate more convenient application of and completion of TMS treatment. For example, a TMS device adapted for home use may eliminate the need for the patient to make periodic or routine office visits to receive treatment. By enabling home use, rTMS procedures may be performed with reduced disruption and inconvenience to the patient, and may provide a more flexible and individualized environment for effective rTMS treatment. One embodiment according to the present invention includes a TMS helmet that can be kept with the patient and used at any desired location (e.g., at home, at a workplace, etc.).

The different therapeutic applications of TMS may require targeting different areas of the brain. Once the appropriate area has been identified, it may be important that the same brain area is targeted across multiple rTMS stimulations or sessions. Conventionally, rTMS is delivered via a hand-held or an articulated arm set by a physician or a properly trained technician so that the stimulation coil targets the desired part of the subject's brain. The specific area of the brain to be targeted is often determined using either scalp measurements (which are notably imprecise and introduce substantial inter-individual variability), or an MRI of the patient's brain in combination with a frameless stereotactic device.

After the identification of the brain area to be targeted, it may be important to ensure that the same brain region is targeted throughout the treatment (e.g., throughout successive sessions). This is typically achieved via monitoring using frameless stereotaxy and/or repeated adjustments by the physician or technician. Chairs have been developed that incorporate the TMS coils, but are problematic, first, because such devices are not portable, and, second, the patient must remain immobilized to prevent movement that might shift the relation of the TMS coils to the patient's head. Applicant has appreciated that a headpiece or helmet fitted to the patient's own head shape and that incorporates TMS coils positioned so as to target the desired brain region in the patient, would both facilitate portability and improve the consistency of coil positioning both during a session and between multiple sessions.

In conventional TMS therapy, identification of the brain area to be targeted as a result of a particular coil placement is often based on a concentric sphere model of the field of distribution of the induced magnetic fields and resulting current fields. Applicant has recognized that such models may be inaccurate, and subsequently result in application of magnetic fields that target (e.g., that are focused and/or stimulate) unintended areas of the brain. Applicant has identified that a more accurate determination of brain target location may be determined by considering tissue characteristics of the individual subject undergoing treatment. For example, brain lesions (e.g., strokes) may markedly disrupt induced fields within the brain. Moreover, anisotropy and/or inter-individual variability may cause changes in the induced fields that are not captured by conventional head-models. Such considerations may cause substantial inaccuracy in computing the target location, for example, reducing the spatial precision in determining focal areas, and/or reducing the efficacy of the treatment and potentially increasing risks and undesirable side-effects.

Applicant has developed methods for guiding the placement of coils by considering characteristics of the tissue within the brain. One embodiment according to the present invention includes determining, at least in part, the distribution of one or more dielectric properties (e.g., conductivity, permittivity, permeability, etc.) in a portion of the brain to facilitate accurate positioning of one or more TMS coils. For example, identified dielectric characteristics of the brain may be used to determine the current density distribution that would result from a particular coil placement. The procedure may be used to determine a coil placement that induces a desired current density to an intended portion of the brain.

In one embodiment according to the present invention, TMS coil placement guided by dielectric property determination may be incorporated into a generally portable TMS helmet, facilitating individualized treatment by, for example, correcting for distortions that brain lesions or other individual brain characteristics impose on the TMS induced currents. However, such coil placement methods may be used with any TMS device, as the aspect of the invention are not limited in this respect.

Even when precision in targeting a specific brain region is high, the locality of effects of TMS may remain suboptimal. However, diffusion spectrum imaging (DSI), can be used to reveal tissue characteristics in order to maximize the focal specificity of the effect of TMS on a given brain region. DSI-guided TMS may be integrated to provide an optimal system to allow increased precision with respect to stimulating the brain with TMS. These solutions may be incorporated into a portable helmet TMS device, or other TMS device, as the aspects of the invention are not limited in this respect.

In conventional TMS therapy, the level (strength) of the magnetic field, the duration of the stimulation, the polarity of the pulse, and the frequency of the stimulation are all selected by the physician or technician, and set according to treatment protocols that generally take some individual factors into consideration and are otherwise disease specific. This setting of stimulation parameters is often manually performed by setting knobs, switches, and/or computer menus on the research instrument prior to applying the rTMS to a specific area on the patient's head. Applicant has appreciated that software may be programmed with instructions to carry out a particular treatment program for a patient. The program may be executed by a portable TMS device to administer the treatment at the patient's home, office, etc.

In addition, Applicant has appreciated that stimulation parameters may be better set if dependant on recorded physiologic parameters of the state of the patient's brain, which may be accomplished using the patients on-line recorded electroencephalograph (EEG). Various TMS techniques guided by EEG are discussed in U.S. Pat. No. 6,571,123 ('123), entitled “Method and Apparatus for Recording an Electroencephalogram During Transcranial Magnetic Stimulation,” which is herein incorporated by reference in its entirety. One embodiment according to the present invention includes incorporating EEG techniques into a TMS helmet adapted for general portability.

Following below are more detailed descriptions of various concepts related to, and embodiments of, methods and apparatus according to the present invention. It should be appreciated that various aspects of the invention described herein may be implemented in any of numerous ways. Examples of specific implementations are provided herein for illustrative purposes only.

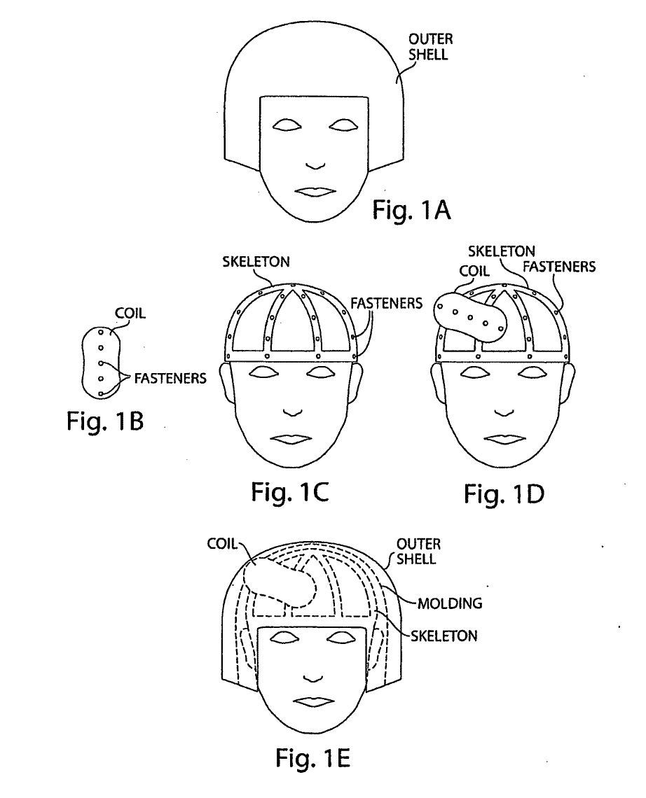

FIGS. 1A-E illustrate a headpiece, in accordance with one embodiment of the present invention. The headpiece includes an outer shell (shown in FIG. 1A) that houses various components that together facilitate performing one or more TMS procedures on an individual. As discussed above, placement of TMS coils varies depending on the procedure and the individual. For example, different treatments target different portions of the brain. In addition, individual patients have different sized heads, which may need to be considered in the construction of an individualized headpiece or helmet.

FIGS. 1B-1E illustrate one embodiment of a headpiece that can be individualized for a particular patient's treatment. FIG. 1B illustrates a TMS “figure-eight” coil similar to coils ubiquitously used in TMS procedures. However, the TMS coil includes a number of fasteners (e.g., snaps) that allow the coil to engage with an inner portion or skeleton of the headpiece that has been individualized for a particular patient's head. The fasteners may be of any number and of any type, as the aspects of the invention are not limited in this respect.

FIG. 1C illustrates an inner portion, referred to as a skeleton, according to one embodiment of the present invention. The skeleton may be made, for example, from a moldable plastic, or other material that can be shaped to an individual's head. Alternatively, the skeleton may simply come in a number of predetermined sizes that would allow the skeleton to fit a wide variety of head sizes. The skeleton may be formed from a number of strips, each strip including any number of fasteners adapted to engage with the fasteners on the TMS coil.

A physician or trained operator may determine the location on the head that the coil should be placed to carry out the intended treatment. The coil may then be fastened into place by engaging one or more (preferably two or more fasteners to prevent rotation) of the fasteners located on the coil and the fasteners located on the skeleton. It should be appreciated that any number of strips and any number of fasteners on the strips and/or coil may be used to ensure that the desired location has available fasteners to hold the coil at the desired. For example, any number cross strips may be added with fasteners to achieve a desired granularity with respect to the number of possible positions for the coil.

Alternatively, the location of the coil may be determined beforehand and the skeleton formed in accordance to make sure the precise intended location has available snaps for positioning the coil. For example, after the intended position of the coil has been determined, fasteners and/or strips may be positioned and arranged to form a skeleton that can engage the TMS coil at precisely the intended location. FIG. 1D illustrates the coil fastened to the skeleton at a position computed to carry out a particular treatment on an individual.

After the TMS coil has been fastened to the skeleton, the outer shell may be positioned over it to secure the coil and provide a protective barrier so that the patient cannot disturb the coil during and between treatments. In one embodiment, the outer shell contains a layer of moldable plastic, for example, material of the type used to make dental impressions or used in athletic mouthpieces. The outer shell may be heated so that the moldable plastic layer or molding becomes soft and pliant. The skeleton, with the attached coil may then be pressed into the molding. When the molding cools and hardens, the coil will be implanted in the molding and held firmly in place with the individualized and/or custom-fit skeleton. The hardened molding provides additional security against movement of the coil and provides an interface between the outer shell and the inner skeleton to form an integrated and personalized TMS headpiece.

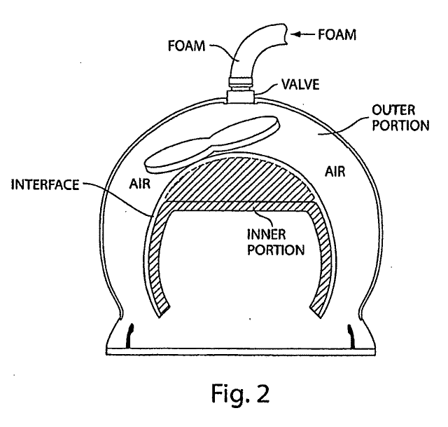

It may be advantageous to be able to reuse an outer portion of a headpiece (including the TMS coil) and have a disposable inner portion custom fit to a patient's head. FIG. 2 illustrates such a headpiece, in accordance with another embodiment of the present invention. The headpiece in FIG. 2 includes an inner portion that may be of any material that can be generally custom fit to an individual's head, such as foam rubber, moldable plastic, or any of the various padding materials used in athletic helmets such as football or motorcycle helmets. The headpiece also includes an outer portion formed from a generally rigid material having a hollow inner core. The inside of the outer portion and outside of the inner portion are designed to interface with one another, for example, as the padding in a football helmet interfaces with the hard outer shell.

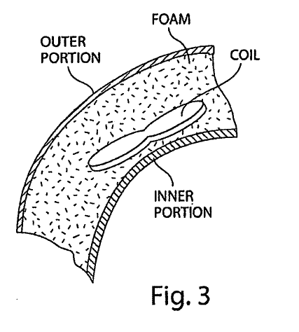

The headpiece also includes a valve adapted to connect to a hose or other foam applicator. Once the coil has been correctly positioned within the outer portion, foam may be applied through the valve to fill the open space in the hollow outer shell to hold the coil firmly in place. FIG. 3 illustrates a cross-section of the headpiece after the foam as been added. The foam may be a material that hardens to hold the coil in place, or may be any suitable material for securing the coil. In an alternative embodiment, the valve may be adapted to engage with a vacuum pump. After the coil has been positioned, the air may be pumped from the hollow shell to form a vacuum. The outer shell may be made from a material such as a plastic, that will collapse in a vacuum to firmly hold the coil in place (e.g., as in done in vacuum packing).

In another embodiment, the outer portion includes a number of moveable tracts to which the coil is attached. The tracts are arranged to give the coil a desired number of degrees of freedom and may be adapted to lock into place once the desired position has been reached. Any number of methods for positioning and securing one or more RF coils to a headpiece may be used, as the aspects of the invention are not limited in this respect.

As discussed above, there are treatments for a variety of different conditions and disorders. The treatments may vary in the duration of the stimulation, frequency of treatment, frequency and power level of the emitted fields, etc. In conventional TMS therapy, the variety of treatment types do not pose a difficulty because the patient must visit the hospital or doctors office, where the appropriate procedure may be performed by the doctor or trained operator. However, various aspects of the invention are directed to portable headpieces that may be used by the patient at home. Accordingly, at least some of the know-how of the doctor and/or operator may need to be incorporated into the headpiece.

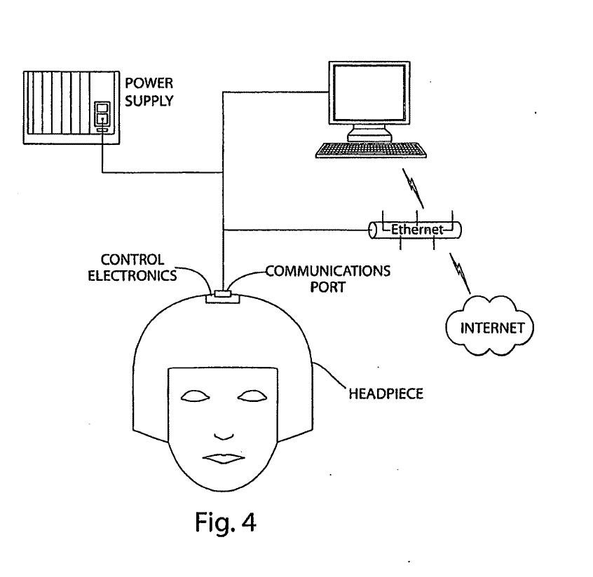

FIG. 4 illustrates a headpiece in accordance with one embodiment of the present invention. The headpiece illustrated in FIG. 4 includes a communications port having one or more electrical connections. For example, communications port may have a connection that allows the headpiece to connect to a local PC, to an Ethernet port and/or the Internet. In addition, the communications port may have a power connection that allows the headpiece to be connected to a power supply capable of generating power sufficient to drive the TMS coil to effect the desired TMS treatment. The headpiece may also include control electronics for controlling the application of power and the timing of the treatment. The control electronics may be hardware, firmware, software or a combination thereof, as the aspects of the invention are not limited in this respect.

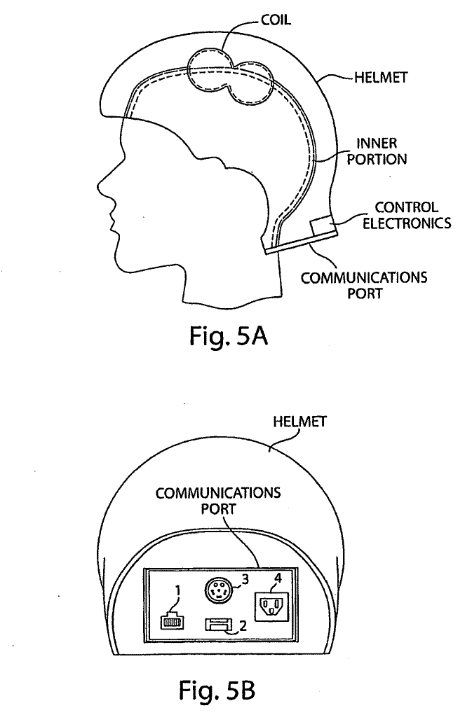

FIG. 5 illustrates a headpiece adapted for communications, in accordance with another embodiment of the present invention. The headpiece illustrated in FIG. 5 may include any of the components, positioning and/or securing mechanisms described above or any other positioning and securing means suitable for providing a headpiece that can be custom-fit for a particular patient's TMS treatment. FIG. 5A illustrates a side-view of the headpiece, including a communications port and control electronics integrated into the back portion of the headpiece. FIG. 5B shows the headpiece viewed from the bottom to better illustrate the communications port and control electronics.

The communications port includes a standard Ethernet connection 1 so that the headpiece may be connected to the Internet or to a local computer. The communications port also includes a Universal Serial Bus (USB) port 2 that can be used to connect the headpiece to standard personal computers or other peripheral devices, as discussed in further detail below. The communications port also includes a power connection 3 that can be used to connect to a power supply adapted for, or capable of, generating power sufficient to energize the coil at any of the standard TMS power levels used to provide stimulation. In addition, a standard power connection 4 may be used to connect to, for example, 60 Hz wall power to power, for example, any cooling mechanisms that the headpiece may require.

The Ethernet and/or USB port allows the headpiece to be connected to one or more programs configured to control the particular treatment of the patient in possession of the headpiece. For example, software installed on a local PC and programmed specifically for the patient's treatment may be provided to the control electronics to administer TMS sequences appropriate for the corresponding treatment. In addition, control electronics may also report back to the local PC about status or other information (such as verifying that the patient completed a session) that can be monitored remotely (e.g., via the internet) and adjusted if necessary by a physician without requiring the patient to make an office visit.

In one embodiment, a physician determines what should be the treatment for a particular patient and inputs that information to a program configured to administer treatments according to information provided by the physician, e.g., duration of each session, number of sessions per day/week/month, power level, pulse sequences, and/or patient information, diagnostic information etc. The customized program may be saved on a portable storage device such as a jump drive or flash drive, or other computer readable medium adapted to connect to, for example, the USB port of the headpiece. The control electronics may include one or more processors adapted to execute instructions saved on the portable storage medium. Accordingly, a physician can provide a customized treatment program that can be delivered via a portable storage device and connected to the headpiece to administered the saved treatment programs.

The headpiece may also be adapted to only operate via a control program (e.g., particular duration and session frequency, power levels, etc.). In this way, the patient cannot misuse the headpiece and administer TMS sessions that have not been approved and prescribed by the doctor. The headpiece may cease to operate after the treatment duration has expired, and may, for example, have an internal clock by which it will only operate according to the programmed treatment schedule. In addition, password information, fingerprint, voice and/or retinal analysis may be used so that only the targeted patient may use the device.

In addition, diagnostics can be obtained from the headpiece and transmitted to the physician to monitor the treatment. In one embodiment, EEG feedback is used to customize and/or optimize the treatment based on the electrical information of the patients brain. For example, stimulation parameters may be set depending on the real-time physiologic parameters of the state of the patient's brain, thus guiding the patient's treatment according to the patient's recorded EEG, as discussed in further detail below. Other monitoring procedures can be done by the physician by real-time data obtained from the headpiece and transmitted over a network, e.g., the internet, via the communications port.

As discussed above, proper placement of a TMS coil may be an important factor in the effectiveness of TMS treatment. The identification of the brain area targeted by the placement of the TMS coil is conventionally performed using a concentric sphere model of the field distribution of the induced magnetic fields and resulting current fields. However, these models do not consider the tissue characteristics, anisotropy and inter-individual variation. Applicant has recognized that the dielectric properties (e.g., conductivity, permittivity, permeability, etc.) may disrupt the induced fields resulting from the TMS coil. As a result, the TMS coil may be positioned to stimulate the incorrect or unintended regions of the brain, limiting the efficacy of the treatment and increasing the risk of undesirable side-effects.

Applicant has appreciated that identification of the brain area targeted by a TMS coil that incorporates tissue characteristics, may improve the precision of TMS coil placement. In one embodiment of the present invention, tissue conductivity characteristics obtained from a subject's MRI are used to determine the placement of the TMS coil to stimulate a desired region of the brain. In another embodiment, one more dielectric properties of brain tissue are obtained from direct measurements of the subject's brain and used to determine the placement of the TMS coil to stimulate a desired region of the brain.

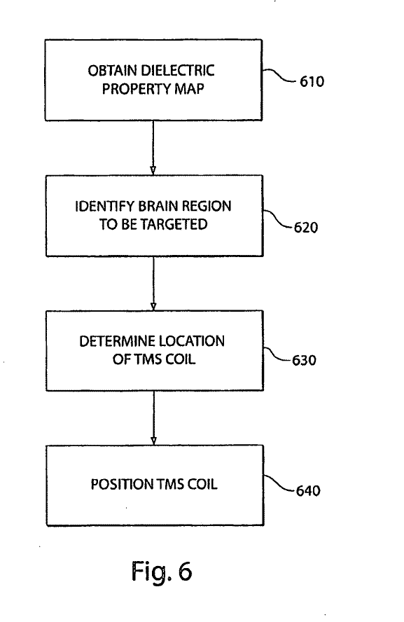

FIG. 6 illustrates a method of positioning a TMS coil, in accordance with one embodiment of the present invention. In step 610, a dielectric property map of a subject's brain, or portion of the subject's brain is obtained. The term map refers herein to any information that associates a location in space with a value of one or more properties. Accordingly, a dielectric property map may be information that associates brain locations with one or more values of dielectric properties at the corresponding location. Various methods are known to obtain dielectric properties, such as from existing tissue property tables obtained from direct measurement, from MRI measurements of the subject's brain, various impedance mapping techniques, etc. Any one or combination of methods may be used to obtain a dielectric property map of the subject's brain or portion of the brain.

In step 620, the region of the brain to be targeted is determined. As discussed above, the region of the brain to be targeted typically depends on the type of treatment the subject is undergoing. In particular, the region of the brain to be targeted will depend largely on the condition that is being treated. Many types of conditions have been identified that may be treated by TMS, and the brain region to be targeted depends, at least in part, on the portion of brain responsible or associated with the condition being treated.

In step 630, the placement of the TMS coil is determined based on the region of the brain to be targeted and the dielectric property map. As discussed above, Applicant has identified that the dielectric properties of brain tissue affect the magnetic field distribution. As a result, to determine the precise placement of the TMS coil, changes in the magnetic fields due to the dielectric properties of the brain may need to be considered.

Applicant has developed methods of using the dielectric property map to compute the location of TMS coil placement to stimulate the intended region of the brain. Various methods of determining TMS coil placement based, at least in part, on one or more dielectric properties of brain tissue are described in detail in the Appendix. In particular, a model of the magnetic field distribution in the dielectric environment of the subject's brain may be used to compute the correct location of the TMS coil to stimulate the intended targeted area of the patients brain. The methods provided in the Appendix are merely exemplary, and any method of computing TMS coil placement based, at least in part, on one or more dielectric properties may be used, as the aspects of the invention are not limited in this respect.

Applicant has appreciated that direction dependent changes in conductivity (i.e., conductivity anisotropy) have an impact on the predicted site and magnitude of the maximum cortical current density. In the presence of anisotropies, as seen in both healthy and pathologic brain tissue, relatively large conductivity dependent changes occur in the vector components of the induced current densities throughout the brain tissue, most prominently at the gray matter-white matter interface. Applicant has recognized that such direction dependent changes in conductivity can be discriminated via DSI imaging. DSI additionally provides a method to map principal white matter tracts in the brain and to resolve cyto- and myelo-architectonics of the cortex. This provides a method to investigate the orientation specificity of TMS, and allows comparison of TMS with electromagnetic models of the brain that include interactions with neuronal sub-populations in gray matter and subcortical white matter resolved with DSI.

Accordingly, Applicant has developed methods of improving guidance of TMS coil placement by including information about the direction dependent changes in conductivity, for example, derived from DSI measurements. Accordingly, field models used to determine coil placement from one or more dielectric properties of a portion of the brain may be extended to incorporate conductivity anisotropy and fiber tract information to improve the TMS coil placement algorithms.

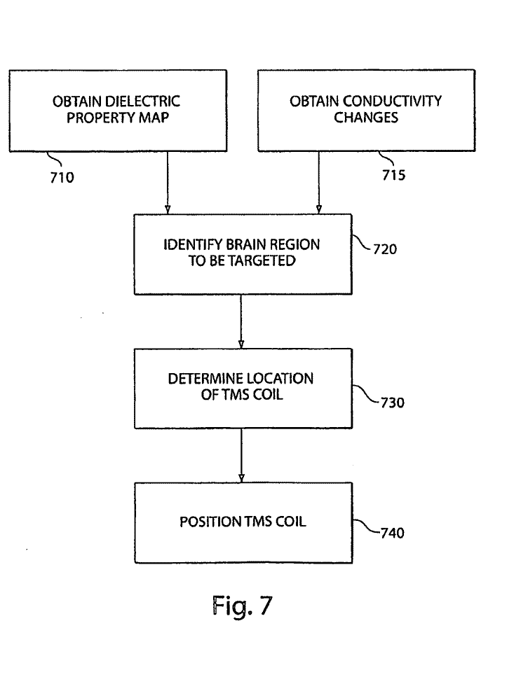

FIG. 7 illustrates a method of positioning a TMS coil, in accordance with another embodiment of the present invention. The method illustrated in FIG. 7 may be similar to the method illustrated in FIG. 6. However, in step 615, conductivity anisotropy measurements and/or fiber tract information is obtained from one or more DSI images. Accordingly, in step 730, both the dielectric property information and the conductivity anisotropy measurements and/or fiber tract information are used to determine the appropriate location of the TMS coil. Various methods of using conductivity anisotropy and/or fiber tract information, for example, obtained from DSI images is discussed in further detail in the Appendix.

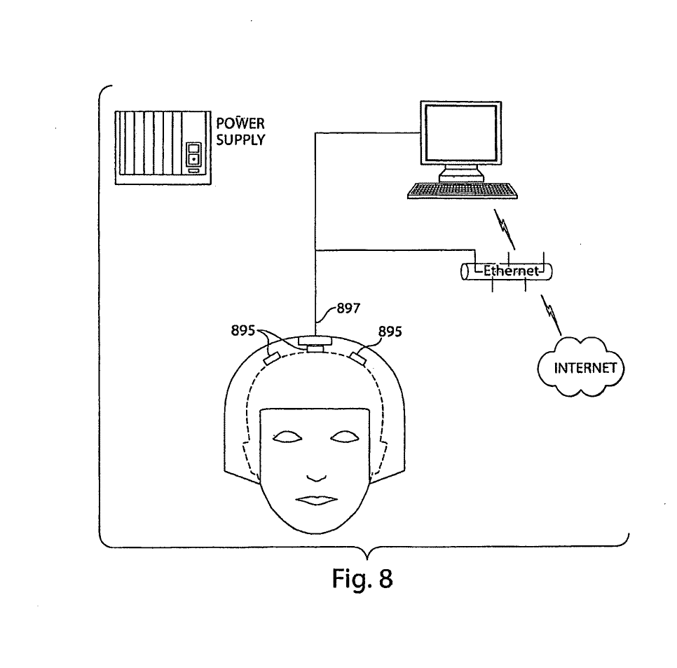

FIG. 8 illustrates a portable helmet having EEG probes incorporated into the helmet, in accordance with another embodiment of the present invention. In particular, electrodes 895 are positioned within the helmet to contact the subject's head. EEG signals are transmitted over communications means 897 to control electronics and/or over the network to a remote location. The EEG signals may be automatically processed to adjust the TMS protocol based on the current state of the subject's brain, or the EEG signals may be analyzed remotely by a clinician who may provide control signals back to the helmet to optimally control the TMS protocol.

As discussed in the incorporated '123 patent, combined EEG and TMS may be used to maximize the efficacy of TMS treatments. Applicant has appreciated that the temporary state of activation of the subject's brain may impact both local and distant effects of TMS stimulation. Accordingly, by monitoring brain activity via EEG, the TMS protocol may be optimized real-time based on the subject's current brain state. The effects of TMS depend on the parameters of stimulation and on the specific brain region targeted. Applicant has appreciated that the impact of TMS spreads, from the directly targeted brain region along cortico-cortical and cortico-subcortical connections to influence a distributed cortico-subcortical, bi-hemispheric neural network.

Applicant has appreciated that the efficacy of TMS treatment may be improved by tailoring the TMS protocol (e.g., the stimulation parameters) based on EEG information. In particular, one or any combination of the level (strength) of the magnetic field, the duration of the stimulation, the polarity of the pulse, and the frequency of the stimulation may be adjusted based on the current state of the subject's brain as indicated by the EEG signal provided by electrodes 895 incorporated into the portable helmet illustrated in FIG. 8.

Below are provided various studied conducted by the Applicant that present finding related to various embodiments according to the present invention. Requirements recited in the following examples and applications of the various aspects of the invention pertain only to the particular embodiment being described and do not express limitations on the invention as a whole. The data, features, components and implementations in the following studies are mentioned merely to provide examples of the various aspects of the present invention. However, the aspects of the invention are not limited to the descriptions in the following studies.

In addition, the intended benefits discussed in the following studies are not requirements or limitations of or on the invention, but rather mention an intended effect or benefit of certain embodiments of the present invention, though certain embodiments may not have the intended effect and/or benefit. In addition, any described shortcomings or stated requirements should be construed as pertaining only to a particular embodiment only, and not to the invention as a whole. The description and illustrations presented in the following studies are by way of example only.

As discussed above, TMS is a non-invasive procedure that utilizes magnetic fields to create electric currents in discrete brain areas. TMS involves discharging the energy stored in a bank of capacitors through a coil of copper wire that is held over the subject's scalp. The current pulse flowing through the coil generates a rapidly fluctuating magnetic field that penetrates the scalp and skull virtually unimpeded and induces a current in the cerebral cortex below the coil. The strength of the induced current is a function of the time rate of change of the magnetic field, the tissue electromagnetic properties, and the relative coil to head position. The physiologic response is caused by current flow in the cortical tissue, which leads to neuronal depolarization.

TMS has a number of applications in clinical neurophysiology and neurology, many examples of which were discussed in the foregoing. Trains of repetitive TMS (rTMS) of appropriate frequency, intensity, and duration can lead to transient increases or decreases in excitability of the affected cortex that last beyond the duration of the train itself. rTMS has been found to be a promising noninvasive treatment for a variety of medical conditions. The number of applications continues to increase and there are a large number of ongoing clinical trials investigating the use of TMS for a variety of diseases.

Therapeutic utility of rTMS has been claimed in the literature for psychiatric disorders, such as depression, acute mania, bipolar disorders, hallucinations, obsessions, schizophrenia, catatonia, post-traumatic stress disorder, or drug craving; neurologic diseases such as Parkinson's disease, dystonia, tics, stuttering, tinnitus, spasticity, or epilepsy; rehabilitation of aphasia or of hand function after stroke; and pain syndromes, such as those caused by migraine, neuropathies, low back pain, or internal visceral diseases like chronic pancreatitis or cancer. Therefore the potential significance of rTMS is huge, affecting a large number of patients with debilitating conditions. However, precise targeting of the brain is crucial in order to achieve the desired effects and avoid side effects.

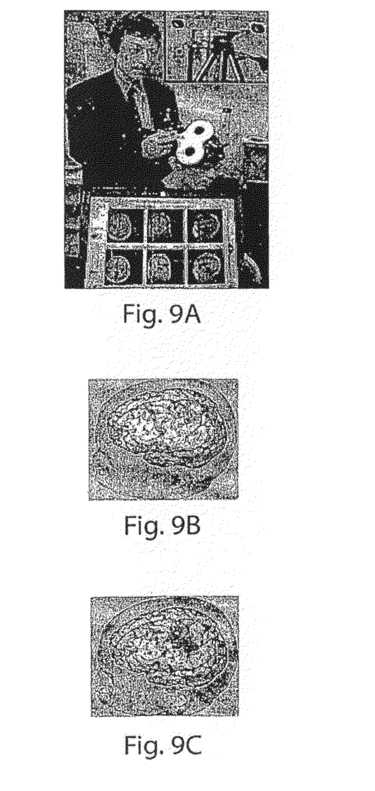

The current standard for defining the location of neural stimulation during TMS is image guided frameless stereotaxy. The frameless stereotaxic systems rely on subject MRI data and coil geometry to digitally track the coil position relative to subject's head and register the predicted stimulation location in MRI space. FIG. 9, image A illustrates the use of a frameless stereotactic system for TMS that is one of several commercially available systems that use the subject's anatomical brain MRI information to guide the placement of the TMS coil on the subject's head, and track in real time the targeted brain region. Functional information can be overlaid on the anatomical MRI and thus be used to define the placement of the TMS coil.

Illustrations B and C of FIG. 9 demonstrate the region of the brain targeted over the course of an rTMS session of 1600 stimuli when scalp markings are used to try to keep the TMS coil steady and precisely targeting a given brain region. Illustration B demonstrates the much greater consistency and precision when the information of the frameless stereotactice system is employed. Displayed in both cases are the site of intercept of the main vector of the induced current with the brain surface. Using the frameless stereotactic system (B) resulted in significantly lower variability in the induced motor potentials. The stimulation point is predicted from theoretically derived free space electromagnetic field solutions based on the coil geometry, essentially assuming the stimulation area to be directly under the coil location where the current is greatest within the coil.

The use of such frameless stereotactic systems represents a major improvement on earlier methods that predicted the targeted brain region on the basis of scalp markers. The use of a frameless stereotactic device provides a reliable means to assure that the main vector of the induced current targets reliably the same brain region throughout a stimulation session and thus minimizes the variability of the induced effects. However, commercially available frameless stereotactic systems completely ignore the electromagnetic interaction between the stimulating fields and the tissues that comprise the physical site of stimulation, and provide no information as to the actual stimulating current distribution induced within the subject's cortex.

The effects of tissues on the stimulating field and the predicted site of stimulation have been analyzed. Computer assisted design (CAD) models have been designed by Applicant based on anatomical MRI subject data and the electrical properties of skin, skull, CSF, gray matter, and white matter under TMS stimulation frequencies. Numerous systems were analyzed by Applicant based on varied anatomical MRI data, pathological abnormalities, and tissue electrical properties. From these studies, Applicant has recognized that the standard predicative methods for ascertaining the stimulation location may be erroneous and may need to be fine-tuned to include the electromagnetic tissue interactions.

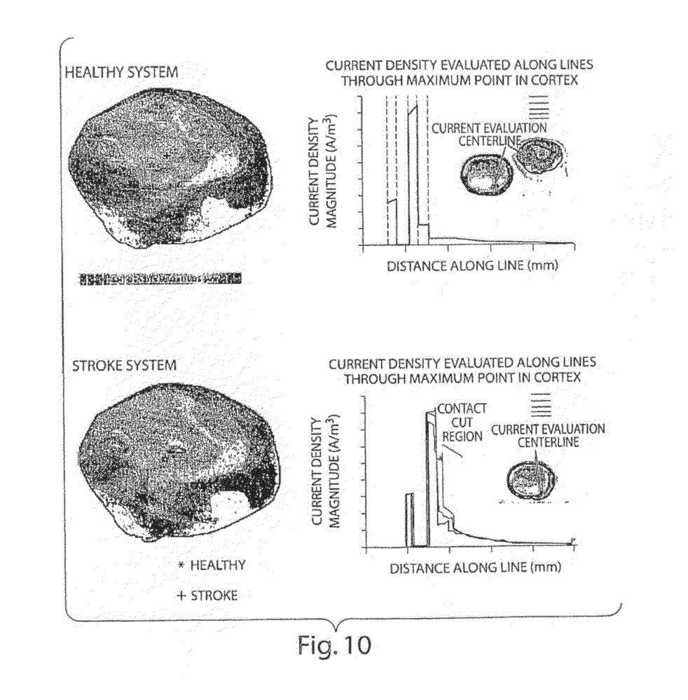

The shortcomings of standard predictive methods are most apparent when one compares solutions from a healthy head system to those of the same head system that includes the effects of a cortical stroke. For instance, in FIG. 10, the induced current density solutions for a healthy head and stoke system are plotted on the cortical surfaces of their respective models. In the healthy head system, the maximum cortical density, of 2.9 A/m², was found directly under the point where the current was greatest in the stimulating coil as would be predicted by standard methods. However, in the stroke model the location of the maximum current density was shunted to the border of the infarction site (1 cm away from the predicted location in the healthy case) and found to increase in magnitude at the infarction edge (with a value of 4.0 A/m²). The CSF provided a shunting path for the stimulating current away from the expected site effectively focusing the current towards the infarction boundary.

Furthermore, the current density magnitudes show little consistency at the region of the stroke damage. In FIG. 10, the current density magnitudes are shown as evaluated along parallel lines that penetrate the model through all the included tissues in each system (skin, skull, CSF, gray matter, and white matter). The evaluation lines were placed to intersect and surround the maximum current density location in the normal case (every +/−1 mm for 5 mm; to highlight the position of the evaluations lines, a transverse slice of the current density for each model is included with the location of the centerline indicated). The current density magnitudes showed little variation around the location of maximum induced current in the healthy case. However, current density magnitudes show erratic stair step jumps in the region of the stroke (over 30% at the region of the infarction boundary) and do not follow a consistent pattern indicative of the healthy case.

Therefore, the predicted site of the stimulation with TMS is not in agreement between the stroke and normal case. These problems are not limited to the case of stroke, but do in fact also appear in considering other pathologies (for example cortical malformations, tumors, vascular malformations, etc.). In fact, brain atrophy alone, on the basis of the increased cerebro-spinal fluid-filled spaces (and thus the increase in current shunting) leads to significant distortions of the induced brain currents. Therefore, it has been appreciated by Applicant that conventional methods for ascertaining the stimulation location may be fine-tuned to include the electromagnetic tissue interactions.

Direction dependent changes in conductivity can be discriminated via DSI imaging, and Applicant has shown that these changes can have an impact on the predicted site and magnitude of the maximum cortical current density. In the presence of anisotropies, as seen in both healthy and pathologic brain tissue, we show large conductivity dependent changes in the vector components of the induced current densities throughout the brain tissue, but most prominently at the gray matter-white matter interface. DSI additionally provides a method to map principal white matter tracts in the brain and to resolve cyto- and myelo-architectonics of the cortex. This provides a method to investigate the orientation specificity of TMS, and allows us to compare TMS with electromagnetic models of the brain that include interactions with neuronal sub-populations in gray matter and subcortical white matter resolved with DSI.

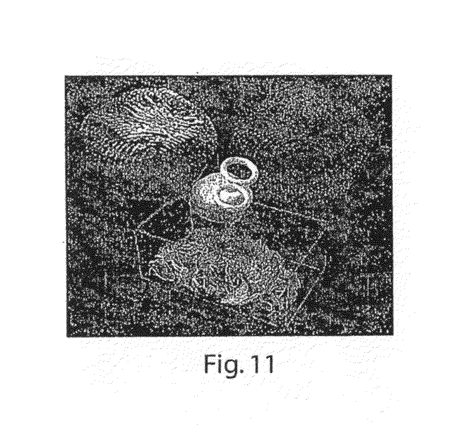

FIG. 11 illustrates an example of the electromagnetic current density fields revealed via the field solver with the underlying white matter architecture as revealed via DSI. Therefore, Applicant has developed improved MRI based field solver and incorporate DSI anisotropy and fiber tract information into our field solver to improve functional guidance for the TMS coil placement.

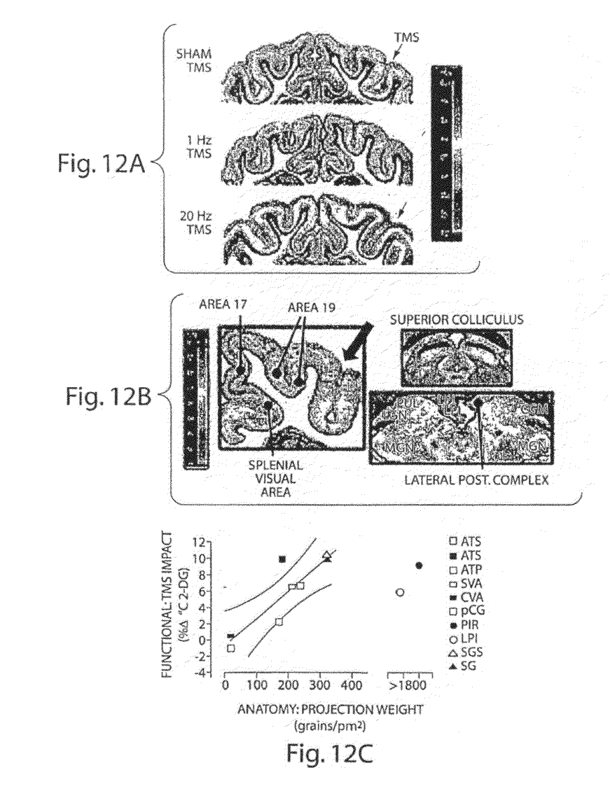

The effects of TMS depend on the parameters of stimulation and on the specific brain region targeted. However, it is important to recognize that the impact of TMS spreads, from the directly targeted brain region along cortico-cortical and cortico-subcortical connections to influence a distributed cortico-subcortical, bihemispheric neural network. In humans, effects of TMS distant to the target site can be documented through studies that combine TMS with positron emission tomography (PET), functional magnetic resonance imaging (fMRI) or electroencephalography (EEG). Applicant has used animal studies, which provide the opportunity of much greater control of stimulation condition and data analysis with much greater detailed information to learn about these network effects. FIG. 12 illustrates findings across a number of studies that have allowed to reach three main conclusions:

First, the effects of TMS on the targeted brain region depend critically on the frequency of stimulation and are different during the time of TMS application (ON-LINE) and thereafter (OFF-LINE). There is inter-individual variability to this, but fundamentally: (a) during the rTMS there is suppression of ongoing brain activity (induction of a ‘virtual lesion’) and the degree of suppression increases with increasing stimulation frequency; (b) after rTMS (off-line) having been exposed to slow (1 Hz) rTMS results in lasting suppression while having been exposed to fast (≧20 Hz) rTMS leads to a lasting facilitation through shifts in intracortical inhibition and facilitation.

Second, there are highly specific distant effects of rTMS to a given brain region, which depend precisely on anatomical connectivity patterns and are correlated with the strength of these connections. These insights allow the prediction, on the basis of knowledge on brain connectivity, of the impact of TMS on neural networks, and ultimately on behavior. Third, work comparing findings in animals at various levels of anesthesia or diverging levels of cortical activation strongly suggests a critical link between the network effects of TMS (and thus the behavioral impact) and the temporary state of activation of the targeted brain region at the time of stimulation. This suggests the need for real-time monitoring of the state of brain activity in the targeted brain area when TMS is being applied. Such insights led us to develop a system to be able to guide TMS with real-time EEG in humans.

TMS-induced neuronal activity spreads beyond the directly stimulated area to anatomically connected sites and thus TMS ultimately induces a modulation of a specific, cortico-subcortical, bihemispheric neural network. Behavioral effects of TMS over a given brain area reflect how the distributed neural network (and the rest of the brain) reacts and compensates for the transient ‘virtual lesion’ during task execution. In other words, the behavioral effects of TMS critically depend on anatomical and functional connectivity of the stimulated area, on the excitatory and inhibitory interplay between target area and connected sites while subjects carry out a given task, on the orchestration of serial and parallel processes across the regions operating in concert for task execution, and on the possibility to tap on functions whose neural bases were left unaffected by TMS.

Therefore, precise control of the behavioral effects of TMS in a given individual may include: (1) precise and consistent targeting of a defined brain region, and (2) timing the stimulation and setting the stimulation parameters so as to guide activity in the targeted brain region and its connected neural network in a predictable and desired fashion. Applicant has developed methods to precisely and consistently target a defined brain region and a specific individual subject. Timing the stimulation and setting its parameters so induce a defined modulation of activity in a distributed neural network may be facilitated by online monitoring of the brain activity.

Applicant has developed an electroencephalographic (EEG) device and protocol that allows recording of electrophysiological signals generated by the human brain during transcranial magnetic stimulation (TMS) despite the TMS-induced high-voltage artifacts. Hardware components that facilitate include slew-rate limited preamplifiers to prevent saturation of the EEG system due to TMS. The protocol involves artifact subtraction to isolate the electrophysiological signals from residual TMS-induced contaminations. In a series of studies, Applicant has illustrated the TMS compatibility of the protocol for studies employing single-pulse TMS designs, as well as those utilizing repetitive TMS.

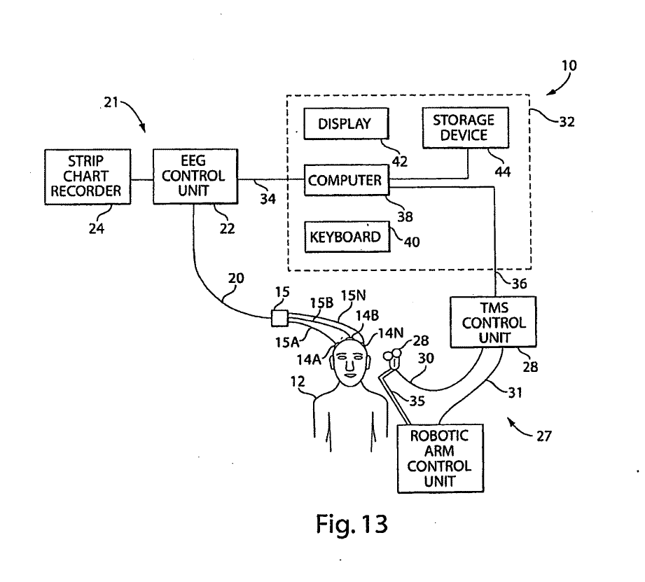

Combined EEG-TMS techniques provide neuroscientists with a unique method to test hypothesis on functional connectivity, as well as on mechanisms of functional orchestration, reorganization, and plasticity. The system is also of potential use for EEG monitoring during repetitive TMS, when TMS parameters fall close to the recommended safety guidelines. Furthermore, EEG guidance of the TMS parameters can provide a means to optimize the timing of the TMS on the basis of the subject's temporary state of brain activity and thus maximize the achieved behavioral impact. FIG. 13 illustrates a system for EEG-controlled TMS, described in U.S. Pat. No. 6,571,123, which is herein incorporated by reference in its entirety.

In summary, TMS provides a valuable tool to modify activity in a targeted brain region non-invasively and influence activity along a distributed cortico-subcortical neural network so as to induce specific behavioral effects in normals and desirable therapeutic effects in patients with a variety of neuropsychiatric conditions. The effects of TMS depend on the stimulation parameters and the interaction between the applied stimulation and the ongoing brain activity. Precise definition of the site of stimulation may be crucial to assure predictable and consistent behavioral effects. Commercially available frameless stereotactic systems provide means for online monitoring of the targeted brain region and thus maximization of consistency in brain targeting. However, tissue characteristics critically influence the distribution of the TMS-induced current in the human brain and thus sophisticated field solver solutions have to be implemented to be able to reliably predict where the current maxima (and thus the site of stimulation) will be induced for a given stimulation coil and a given coil placement.

Guiding coil placement with DSI-brain imaging and co-registering the DSI information and the field-solver solutions of the TMS-induced currents, allows for maximization of TMS focality and efficacy by accounting for tissue anisotropy and taking into consideration directional specific effects of TMS. Finally, online monitoring of EEG and controlling TMS delivery (timing and stimulation settings) by the momentary EEG activity, provides a means to optimize the TMS impact onto the ongoing brain activity and thus control the specificity and maximize the magnitude of the behavioral effects.

TMS is a promising therapeutic tool for a wide range of highly disabling and prevalent neuropsychiatric disorders. Some embodiments of the present invention may enable safer, more effective, more comfortable and accessible, and more cost-effective TMS treatment by allowing stimulation settings to be tailored to the individual needs, and for the patient to self treat themselves at home as guided by a clinician via telecommunication routes.

Transcranial Magnetic Stimulation (TMS) is a non-invasive procedure that utilizes magnetic fields to induce stimulating electric currents in discrete brain areas. A growing understanding of this technique has led to the development of numerous diagnostic and therapeutic applications. Therapeutic utility of rTMS is likely in a number of psychiatric disorders, neurologic diseases, rehabilitation, and pain syndromes. However, there is a need to improve on the efficiency, safety, and convenience of rTMS.

Common to all therapeutic application of rTMS is the fact that stimulation has to be applied repeatedly for consecutive days (generally 10 to 20 days) in daily or even bi-daily sessions. Under current methodology and practice, this means that patients have to go to the doctor's office or laboratory daily. In addition, following such an ‘induction phase’ there is generally the need for maintenance therapy, so that for example in the case of depression, patients will require weekly to monthly sessions possible forever. This creates a definite inconvenience, and while it may be possible to structure the office visit so as to derive some therapeutic benefit from it, the possibility of delivering the rTMS at the patients' own home is appealing. For example, given the need to visit a doctor's office or laboratory, weekends and holidays cannot be effectively covered and only once per day treatment is often practical.

However, rTMS treatment for medical conditions such as epilepsy and depression may be more effective if delivered 2×, 3×, or 4× per day, or more appropriately administered such as after getting up in the morning or before going to bed at night to maximize interaction with circadian factors. It may also be better for rTMS treatment effectiveness to be delivered for a longer period of time or for short periods of time but more frequently. Therefore, self-delivery of rTMS by the patient in his home environment may be far more effective and certainly would provide a much more flexible and individualizable protocol:

For each therapeutic application rTMS has to be applied to slightly different brain areas, and it is obviously important that the same brain area is targeted across rTMS sessions. Currently, the delivery of the rTMS is via a hand-held or an articulated arm set by a physician or a properly trained technician so that the stimulation coil targets the desired part of the subject's head (and brain). The specific area of the brain to be targeted is determined using either scalp measurements (which are notably imprecise and introduce substantial inter-individual variability), or the patient's own brain MRI via a frameless stereotactic device.

However, identification of the brain area to be targeted at the beginning of the rTMS session is not enough. In addition, throughout the treatment session it is critical to assure that the same brain region is targeted. This may require on-line monitoring using frameless stereotaxy and repeated adjustments by a physician or technician. The development of a chair that incorporates the TMS coils is not sufficient since the patient would need to be completely immobilized to prevent movement that might shift the relation of the TMS to the patient's head. Therefore, the development of a helmet that is fitted to the patient's own headshape and that incorporates the TMS coils placed so as to target the desired brain region in the patient, may provide an improvement over currently existing systems.

The identification of the brain area targeted by TMS is currently at best based on concentric sphere models of the field of distribution of the induced magnetic fields and resulting current fields. Such models fail to fully consider tissue characteristics, anisotropy, and interindividual variability. Brain lesions, particularly strokes, markedly disrupt the induced fields. Such effects can limit the efficacy of TMS and increase its risks and undesirable side-effects. Therefore, the current methods for ascertaining the stimulation location need to be fine-tuned to include the electromagnetic tissue interactions in order to refine the controlled targeting of desired brain regions with TMS and improve current applications.

Applicant has developed methods and apparatus for guiding TMS coil placement on a subject's head by an on-line model of the TMS current density distribution based on the tissue conductivity characteristics of the subjects' MRI. This method can be implemented on enhanced frameless stereotactic systems or incorporated onto novel systems, such as the proposed portable, helmet-like TMS stimulator. This will allow correcting for the distortions that brain lesions or individual brain configuration characteristics impose onto the TMS induced currents. Such distortions result in lack of spatial precision of the TMS and hence limit its therapeutic potential. An independent but related problem is that of the focal precision of TMS. Even when precision in targeting a specific brain region is high, the locality of effects of TMS remains suboptimal. However, diffusion spectrum imaging or DSI, can be used to reveal tissue characteristics in order to maximize the focal specificity of the effect of TMS on a given brain region. This new methodology of DSI-guided TMS will be integrated to provide an optimal system to allow precise stimulation of the brain with TMS.

As currently applied, the level (strength) of the magnetic field, the duration of the stimulation, the polarity of the pulse, and the frequency of the stimulation are all selected by the physician or technician, and set according to treatment protocols that generally take some individual factors into consideration and are otherwise disease specific. This setting of stimulation parameters is manually performed by setting knobs, switches, and/or computer menus on the research instrument prior to applying the rTMS to a specific area on the patient's head. Ideally, it would be optimal to be able to set the stimulation parameters depending on on-line recorded physiologic parameters of the state of the patient's brain. This can be accomplished guiding the TMS by the patient's on-line recorded EEG.

Provided below is the theoretical development of the use of dielectric properties of the brain to guide TMS placement, and the effects on localization of anisotropy and other heterogeneous regions of current density distribution, and empirical studies verifying the use of dielectric properties to guide TMS placement to better target a desired area. The discussion begins with a description of DC stimulation and TMS simulation, and efforts to model the dielectric properties of regions of the brain.

Currently DC stimulation is applied via patch electrodes (surface areas from 25-35 mm²) placed on the scalp surface. Currents usually range in magnitude from 0.5-1 mA and are applied from seconds to minutes. The electrodes can be anything from saline soaked cotton to specifically designed sponge material mixed with conductive gel. There is no complex circuitry comprising the stimulators as numerous configurations exist for constant current sources and current isolators are not needed because the stimulators are battery driven.

In order to examine the current strengths injected into the cortex transcranially we present an intuitive resistor model of the head system with a 7×5 cm anode above the M1 and a 7×5 cm cathode above the contralateral orbital. To calculate the resistances of the tissue, it is assumed that the currents can flow either parallel to the surface of the tissues or axially through the tissues and that the tissue resistances can be estimated as rectangular resistors (Resistance=Length/(Area×σ)) (See FIG. 1.3). The length of the parallel tissue resistors were determined by assuming that the current flowed along the shortest distance from anode to cathode.

Thus for the parallel skin resistor, the resistor length would be 6 cm (the circumferential distance from the M1 to the contralateral orbital along the scalp of a 38 year old man). It was assumed that the current flowed uniformly from electrode to electrode and thus the area could be determined from the thickness of the tissue region and the dimension of the electrode assumed normal to the current flow (determined by the shortest distance). Thus for the skin, the area of the parallel resistor would be 7 cm×0.6 cm, the cross length of the electrode on the skin and the thickness of the skin. For simplicity, the circumferential length was considered to be 6 cm for each of the tissues.

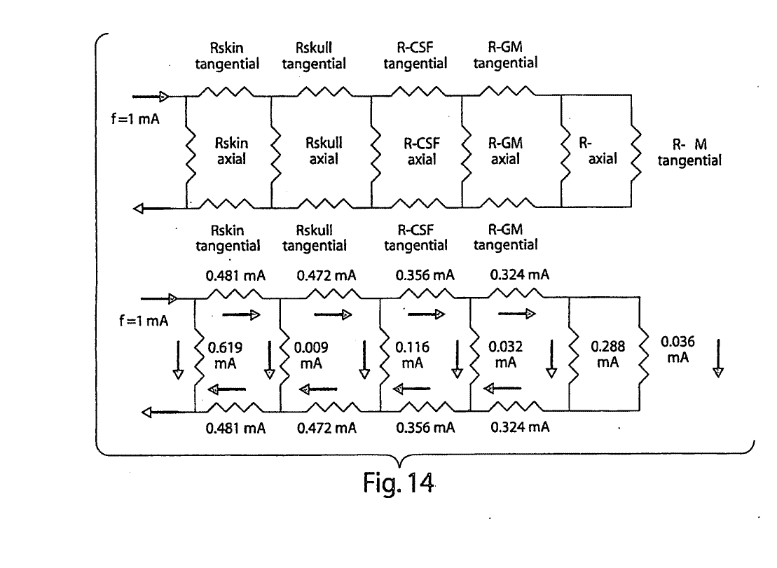

For axial resistance, the electrode area was used as the resistor area and the thickness of the tissue was considered the resistor length. For the white matter, a distance of 6.5 cm was estimated for the distance from electrode to electrode. The values used in the model are displayed in FIG. 14, and tabulated in Table 1 below:

TABLE 1

| Tissue | Conductivity | Thickness | R Parallel | R Axial |
|---|---|---|---|---|
| Skin | 0.47 | 0.006 | 304 | 3.6 |
| Skull | 0.01 | 0.005 | 17100 | 142.9 |
| CSF | 1.75 | 0.003 | 173.2 | 0.51 |
| Gray Matter | 0.28 | 0.005 | 621.1 | 5.2 |
| White Matter | 0.38 | 0.045 | 451.1 | 56.4 |

With the circuit as solved in FIG. 14, the tangential current magnitude in the cortex would be 31.6 μA and the axial current magnitude would be 0.324 mA. Using the areas of the resistors to determine the current densities of each current component, the axial and tangential current densities in the cortex are 0.093 and 0.090 A/m² respectively. These magnitudes are in the same order of magnitude as current densities that alter the level of neural excitability.

Magnetic stimulators typically consist of two main components: a capacitive high voltage (400V− to more than 3 kV) high current (4 kA to more than 20 kA) charge-discharge system and a magnetic stimulating coil that produces pulsed fields of 1-4 Teslas in strength with durations of approximately a millisecond for single pulse stimulators and a quarter of a ms for rapid stimulators. The charge-discharge system is composed of a charging unit, a bank of storage capacitors, switching circuitry, and control electronics. Without the switching circuitry and control electronics, the circuit is essentially a parallel RLC circuit.

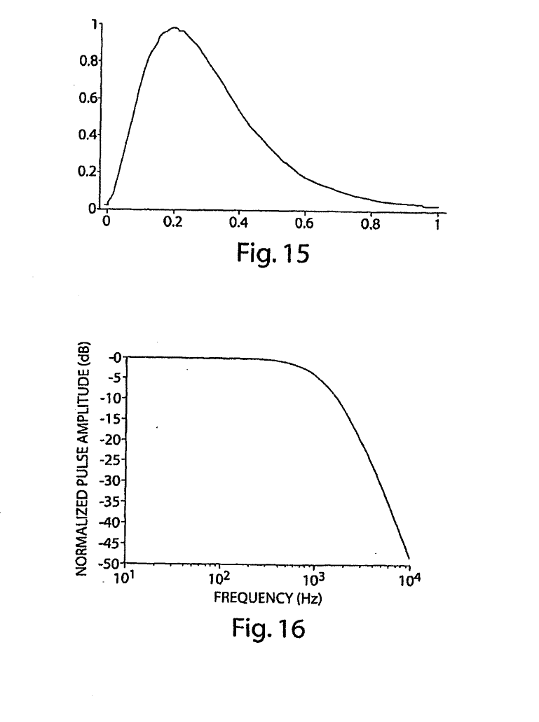

In magnetic stimulators the under damped condition is generally preferred, where R and L are both set to the lowest practical values to minimize heating. In non-repetitive machines reverse charging or ringing in the circuit is prevented by placing a shunt diode across the capacitor, thereby increasing the current decay time and eliminating reverse currents as shown in FIG. 15. With repetitive machines the same essential circuitry remains except modifications are made to the switching system. Clinical stimulators can provide rates up to 60 Hz, yet 20 Hz is not normally exceeded for fear of seizure induction.

Of importance is the fact that the main power components of typical current sources are below 10 kHz. This can be seen by noting that the current source (see FIG. 15) associated with typical single pulse magnetic stimulators can be well fitted by the equation:

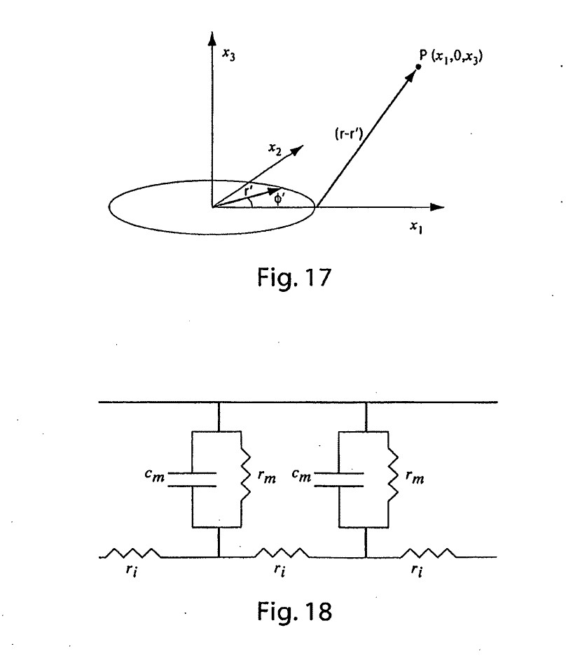

I(t)=(Iₘₐₓe²/T²)t²e^{-2t/T}u(t) (1)

where T≈200 μs. The equation allows for the pulse spectrum to be evaluated in closed form:

$$P ( ω ) = {\int }_{- ∞}^{+ ∞} I ( t ) {e}^{jω t} d t = \frac{( \frac{2 {I}_{max} {e}^{2}}{{T}^{2}} )}{{( 2 / T + jω )}^{3}} \qquad ( 2 )$$

where ω=2πf. FIG. 16 illustrates non-dimensional pulse spectrum of typical TMS current pulse waveforms. The importance of this fact will become apparent in later sections, as it will be assist in justifying the use of quasistatic approximations in the modeling process. For a typical rapid stimulator a typical output current source is slightly more complicated, however the main power component of typical stimulators is seen at around 5 kHz with ringing at the higher harmonics that drops off at more then 60 dB/decade.

Another hardware component of magnetic stimulators is the current carrying coil. Design of the coil may be critically important because it is the only component that comes in direct contact with the subject undergoing stimulation and the coils shape directly influences the induced current distribution and, thus, the site of stimulation. The most common coils used in the clinic are single circular loop or figure of eight shaped. They are constructed from tightly wound copper coils, which are adequately insulated and housed in plastic covers along with feedback temperature sensors and safety switches. Typical coil inductances range from approximately 15 μH to approximately 150 μH, with coil diameters ranging from 4 to 9 cm with anywhere from ten to twenty turns. The figure of eight coil is constructed from two single circular loop coils with inverse current orientations aligned side by side. Although other coil shapes exist, most are just offshoots of the simple circular coils. It should be appreciated that any coil or group of coils may be used, as the aspects of the invention are not limited in this respect.

There are a number of free space field considerations. The magnetic field distribution, H, of a simple circular coil can be calculated via the magnetic vector potential, A, with the use of the current density, J. Noting that:

∇²A=−μJ (3)

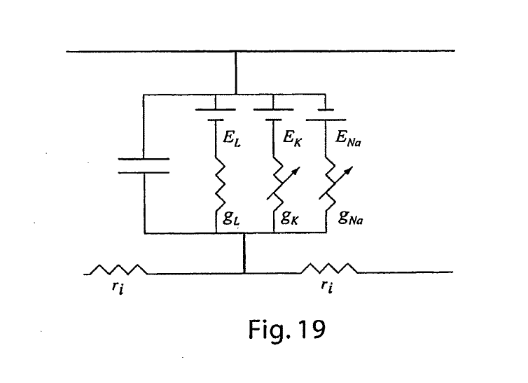

we can use the superposition integral to solve for the magnetic vector potential:

$$A = \frac{μ}{4 π} \int \frac{J ( {r}^{′} )}{\vert r - {r}^{′} \vert} d {V}^{′} \qquad ( 4 )$$

where the prime coordinates (i.e. r′) refer to coordinates along the current source, and the unprimed coordinates (i.e., r) refer to the observation point as illustrated in the coordinate system of FIG. 17. If the radius of the loop is a, J (r′) dV′=I adφ′, x₁′=a cos φ′, x₂′=a sin φ′, and x₃′=0. If we solve the magnetic vector potential at the point P, x₂=0, x₁=ρ (the radial coordinate in the cylindrical coordinate system) and:

$$\begin{matrix}\vert r - {r}^{′} \vert = \sqrt{{( {x}_{1} - {x}_{1}^{′} )}^{2} + {( {x}_{2} - {x}_{2}^{′} )}^{2} + ( {x}_{3} \cdot } \\ = \sqrt{{( ρ - a cos {φ}^{′} )}^{2} + {( a sin {φ}^{′} )}^{2} + {( {x}_{3} )}^{2}} \\ = \sqrt{{ρ}^{2} + {a}^{2} + {x}_{3}^{2} - 2 a ρco s {φ}^{′}}\end{matrix} \qquad 5 )$$

Also note at this point (x₂=0), that the \hat{x}₁ components will cancel due to symmetry conditions, so we can write:

$$A ( r ) = \frac{μ}{4 π} {\int }_{0}^{π} \frac{2 I cos {φ}^{′} a d {φ}^{′}}{\sqrt{{ρ}^{2} + {a}^{2} + {x}_{3}^{2} - 2 a ρco s {φ}^{′}}} {\hat{x}}_{2} \qquad ( 6 )$$

Finally noting, at an arbitrary point, where x₂≠0, finding the \hat{x}₂ component of A is equivalent to finding the φ component. With this fact and the appropriate switch of variables:

$${sin}^{2} α = {cos}^{2} {φ}^{′} = \frac{1 + cos ( 2 {φ}^{′} )}{2} , α = \frac{π - {φ}^{′}}{2} , \\ d {φ}^{′} = - 2 d α , k = \sqrt{\frac{4 ρ a}{{x}_{3}^{2} + {( ρ + a )}^{2}}}$$

we have:

$$A ( r ) = \frac{μ I a}{π} {\int }_{0}^{\frac{π}{2}} \frac{( 2 {sin}^{2} α - 1 ) d α}{( 2 / k ) \sqrt{ρα} \sqrt{1 - {k}^{2} {sin}^{2} α}} \hat{φ} \qquad ( 7 )$$

A_{φ} can be solved for with the use of K (k) and E (k), complete elliptical integrals of the first and second kind, i.e.:

$$K ( k ) = {\int }_{0}^{\frac{π}{2}} \frac{d α}{\sqrt{1 - {k}^{2} {sin}^{2} α}}$$

$$E ( k ) = {\int }_{0}^{\frac{π}{2}} \sqrt{1 - {k}^{2} {sin}^{2} α} d α$$

as:

$${A}_{φ} = \frac{μ Ia}{2 π \sqrt{ρ a}} [ ( \frac{2}{k} - k ) K ( k ) - \frac{2}{k} E ( k ) ] , {A}_{o} \qquad ( 8 )$$

With A_{φ} determined the magnetic field components can easily be determined as:

$$\begin{matrix}{H}_{ρ} = - \frac{1}{μ ρ} \frac{\partial ( ρ {A}_{φ} )}{\partial {x}_{3}} + \frac{1}{μ ρ} \frac{\partial ( {A}_{{x}_{3}} )}{\partial φ} \\ = - \frac{1}{μ} \frac{\partial ( {A}_{φ} )}{\partial {x}_{3}} \\ = \frac{{Ix}_{3}}{2 π ρ \sqrt{( {( ρ + a )}^{2} + {x}_{3}^{2} )}} ( - K ( k ) + \frac{[ {a}^{2} + {ρ}^{2} - {x}_{3}^{2} ] E ( k )}{{( a - ρ )}^{2} + {x}_{3}^{2}} )\end{matrix}$$

$${H}_{x 1} = {H}_{ρ} cos φ$$

$${H}_{x 2} = {H}_{ρ} sin φ$$

$$\begin{matrix}{H}_{x 3} = - \frac{1}{μ ρ} \frac{\partial ( {A}_{ρ} )}{\partial φ} + \frac{1}{μ ρ} \frac{\partial ( ρ {A}_{φ} )}{\partial ρ} \\ = - \frac{1}{μ} \frac{\partial ( ρ {A}_{φ} )}{\partial ρ} \\ = \frac{I}{2 π \sqrt{( {( ρ + a )}^{2} + {x}_{3}^{2} )}} ( K ( k ) + \frac{[ {a}^{2} + {ρ}^{2} - {x}_{3}^{2} ] E ( k )}{{( a - ρ )}^{2} + {x}_{3}^{2}} )\end{matrix}$$

Most importantly, notice that the magnetic vector potential and the magnetic field can easily be calculated for the figure of eight coil (or any combination of circular coils) by using the superposition principle to sum together two simple coil solutions with their origins shifted. And of particular importance in the figure of eight coil is the fact that the magnetic field sums along adjacent coil paths and that both the H field and induced electric field, E, are highest along this point. For a more detailed discussion of these topics see my earlier thesis, which analyzed this in greater detail.

There are few published accounts modeling the fields produced transcranially during tDCs, and of those produced, none capture the contemporary electrode schemes and current densities used for present day clinical stimulation. In 1967, Rush and Driscoll produced a three sphere model of stimulation, but it suffered from computational limitations. Grandori et al produced a model to ascertain the effects of multiple electrodes, finding the ability to focus currents and limited effects of surrounding tissues. However, their results conflicted with the only other contemporary model of tDCs (1993). They produced an MRI based model of transcranial DC stimulation, finding anywhere from 7 to 2 times greater current density magnitude in the skin than in the cortex, but a clear lack of focusing. The differences in the modeling results are indicative of the varied source choices that the authors choose for their studies.

Although more TMS models have been developed compared to the number of tDCs models, there are still relatively few due to the newness of the technique. To date, the models all fall into one of three categories: simplified analytical solutions, numerical solutions to quasistatic forms of Maxwell's equations, or those which make use of a solution method referred to as ‘lead field’ theory, which is solution method based on the reciprocity theorem oftentimes used in Magnetoencephalogram (MEG) and Magnetocardiogram (MCG) studies.

Few simplified analytical solutions exist that do not make use of lead field theory. The unexpected results of an earlier numerical simulation that modeled the induced currents resulting from placing a stimulating coil above an infinite half plane, provided an argument for the absence of electrical field components tangential to the bounding surface for both an infinite half plane conductor and for perfect sphere conductor models representing the human head. These results were shown to be a general result, regardless of the shape or orientation of the stimulating coil.

The argument was based on the fact that for stimulation conditions they concluded that:

∇²E≈0 (13)

where E is the electric field inside the conductor representing the head. Their argument was initially developed for ramp shaped current sources, but was extended to general stimulation conditions, because in the sinusoidal steady state:

∇²E=−ω²εμE+jωμσE≈0 (14)

where both the ωμσ component (∝ inverse (skin depth)²,

$$δ = \sqrt{\frac{2}{ω μ σ}} )$$

and ω²εμ become negligible for the modeled tissue properties from 0 to 10 kHz. In the half plane case, it can easily be seen that the normal component of the electric field has to be equal to zero at the tissue free space interface (assuming the conductivity of the outside region is equal to zero). If we decide to rewrite (14) by making use of the Cartesian coordinate system we can write scalar equations for each of its individual components:

∇²E₁ₓ=0, ∇²E_{y}=0, ∇²E_{z}=

Error! Reference source not found.)

and by assuming the normal component is in the z direction it can be stated that E₃=0 throughout the volume of interest because “if a scalar satisfying Laplace's equation is constant over a closed surface, S, enclosing a volume, V, it has the same constant value throughout V.” A similar argument has been put forward for spherical conductors by showing that the radial component of the electric field should be equal to zero throughout. While the argument was analogous to the half plane case the mathematics are quite a bit more complex due to the fact that the spherical form of ∇² cannot be written easily in scalar form because the Laplacian operates on the unit vectors in the spherical coordinate system.

Thus, in order to prove that there is not a radial component to the electric field, E may be expressed in terms of a vector potential that could be expressed in terms of spherical harmonics. It has been concluded that radial currents in true head geometries, represented by the spherical model, should be minimized relative to the tangential fields, the degree to which depends on the deviation from a true spherical shape.

A model has been formulated predicting the resulting TMS fields based on a solution to the Biot Savaru law in an unbounded space. The model included solving for the electric field, E, by solving the equation:

$$E = - \frac{\partial A}{\partial t} - ∇ Φ \qquad ( 15 )$$

where the magnetic vector potential, A, could be determined via the Biot-Savart law and the scalar potential, Φ, was set to zero, as the calculations were made for an unbounded space in the absence of free charge at TMS frequencies. It was indicated that the gradient of the scalar potential could be accounted for, had the model accounted for more realistic conditions, by solving for the distribution of charge accumulation at conductive boundaries.

Heller and Hulsteyn reviewed the general physics of stimulation and reviewed the general limitations of TMS focality. Under the frequencies of stimulation, they concluded that “no component of the electric field can have a three dimensional local maximum inside any region of constant conductivity”, “the same is clearly true for a spatial derivative of any component and also for the magnitude of the electric field”, and finally the “maxima for all such quantities must be found on a boundary where the conductivity jumps in value.” Both facts result from the Laplacian of the electric field being equal to zero under simplified stimulation conditions.

In 1988 Ueno and his group at Kyushu University in Fukuoka, Japan produced a finite element model of magnetic brain stimulation based on a magnetic diffusion type model developed from earlier hyperthermia studies. The group was consequentially credited with developing the figure of eight coil based on this and subsequent work. However, their early finite element model provided only a particular solution to the proposed problem and did not take boundary conditions into account.

In 1992, Roth et al, produced their three-sphere model of the TMS field distributions and today it is still one of the most accepted models. The model depicts the human head as being composed of a constant conducting sphere representing the human brain, a concentric surrounding shell of constant conductivity representing the skull, and an outermost layer of constant conductivity representing the scalp. This model is similar to one developed in 1969 by Rush and Driscoll for electroencephalogram (EEG) studies.

The three-sphere model accounted for the electrical field induced by stimulation and the secondary field that results from the build up of charge at the conductive boundaries. Their model was developed without the magnetic diffusion equation, but with the realization that at the frequencies of brain stimulation the secondary magnetic fields due to the induced currents will be negligible. Similar to Tofts, they solved (16). Thus, they solved for the electric field in terms of the vector potential due to the coil current source and the Laplacian field. The model used boundary conditions that both the scalar potential and the normal component of the current density (the displacement currents were set equal to zero) were continuous across the interfaces. The group used their model to compare the properties of electrical and magnetic stimulation in a follow up study.

In general, they found an absence of radial electric field components and limited effects of the symmetrically modeled skull and scalp on TMS. However, the majority of the groups work pertains to modeling peripheral nerve stimulation and the cellular mechanisms of stimulation, where they are credited with developing a model for the physics of magnetic peripheral nerve stimulation based upon a modified active cable type model which depicts the spatial derivative of the electric field in parallel to the nerve fiber as responsible for activation.

In 1992, Esselle and Stuchly calculated numerical solutions to (16) assuming the stimulating coil was positioned above an infinite half plane and for a cylindrical volume conductor by first calculating analytical solutions for the volumes by assuming an infinitely short current element and then numerically summing together the results for the components that compose actual stimulating coils. In 1992, De Leo et al, generated the first model that accounted for the asymmetry of the brain and surrounding layers with a finite element model that represented the geometry and conductivity values of the system based on MRI data [54]. They solved for discretized forms of:

$$\begin{matrix}{∮}_{c} E \cdot d 1 = - \frac{\partial }{\partial t} \int B \cdot d S & ( 16 ) \\ ∮ σ ( r ) E \cdot d S = 0 & ( 17 )\end{matrix}$$

and noted that their method's results were in good agreement with those results generated by solving for (16) such as those obtained by Roth et al. However, due to computational constraints they were initially relegated to solving the problem in just two dimensions. In 1995 they extended their work to a third dimension.

More recently in 1996, Scivill, Barker, and Freeston produced a finite element model of the spinal cord that took into account the tissue asymmetries and for the first time the cerebral spinal fluid (CSF). The CSF, which is approximately 4 to 5 times higher in conductivity than the other surrounding tissues of the brain and spinal cord, was shown to effectively attenuate the magnitude of the current density induced in the spinal cord. In 1997, Ueno and his group, now at the University of Tokyo generated a model that accounts for changes in conductivity and the nature of tissue inhomogenities. Using a square model with two dissimilar conductors they have clearly concluded that the conductive boundaries have a constraining effect on the induced current distributions. In the area of magnetic heart stimulation Mouchawar et al, developed a finite element model which included the effects of inhomogeneous conductivities. Following their lead Wang and Eisenberg generated a finite element model and tested it on numerous half plane configurations taking into account the effects of anisotropic conductivities by solving for:

$$∇ \cdot ( \overline{\overline{σ}} ∇ Φ ) = - ∇ \cdot ( \overline{\overline{σ}} \frac{\partial A}{\partial t} ) \qquad ( 18 )$$

with the boundary condition n·J=0 at the enclosing interface and the conductivity, σ̅, represented in tensor form. By accounting for the complex nature of the conductivity they solved for a homogeneous isotropic slab, an inhomogeneous isotropic slab, and a homogeneous anisotropic slab. They determined that the surface charge found from (16) could only be determined after solving for the electric field. These and the other models explained were also reviewed in.

In 2001, Applicant showed the clear implications of tissue heterogeneities and realistic tissue geometries on the induced fields in MQS systems. An additional pathological model was presented which raised many questions and showed that disturbances in the cortical anatomy could lead to alterations in the induced current densities. In 2002, Strazynski et al implemented a more realistic head model and solved the problem with a T-omega solution method, but used a single conductivity for the entire model [59]. In 2003, Nadeem et al presented one of the most comprehensive models of TMS in terms of the actual head model geometry and tissue compartmentalization, but the authors appear to have ignored the tissue permittivities during their use of the three dimensional impedance solution method.

However, even with the shortcomings, other researchers should attempt to follow their lead in including as many tissues as possible in their models. In 2003, Miranda et al studied the effects of heterogeneities and anisotropies in a three-sphere model, clearly showing the important implications of these tissue properties during stimulation. This more recent work underscores the need for the development of more complete computational models to account for the complexities that arise with asymmetric geometries and tissue irregularities.

The final method for analyzing TMS field distributions is to make use of reciprocity and ‘lead field’ theory. Lead field theory was developed in conjunction with interpreting MCG results and more recently with MEG data. As early as 1965 Baule and McFee applied lead field theory to spherically symmetrical volume conductors and proved that radially directed current sources could never generate externally detectable magnetic fields—this is the so called ‘silent zone’ in MEG studies. In 1991 Cohen and Cuffin applied this result to TMS, invoking reciprocity to show that no externally generated magnetic source can produce a radial directed current in a spherical conductor, in agreement with Branston and Togs. Using the results from their earlier studies Cohen and Cuffin modeled current distributions that would be produced by a coil above an infinite half space and obtained results in agreement with other infinite half space models. Another application of lead field theory has been in an evaluation of MEG studies, where Iramina and Ueno showed that the presence of tissue inhomogenities allows for the presence of externally detectable magnetic fields produced by internal radial directed currents.

TMS modeling has advanced significantly since its introduction, but more complete models are necessitated by the non-symmetric nature of the human head and the varied electrical properties of the system. With an increased understanding of the TMS field distributions the mechanism of TMS can be more completely resolved. An understanding of how tissue inhomogenities influence the field is tantamount when predicting stimulation sites for subjects suffering from various pathologies (i.e., strokes, tumors, and trauma) that shift the head even further from the simplified three concentric sphere-model normally used.

With so much still unknown about the electromagnetics of TMS and tDCs it is unfortunate that even less is known about the neural mechanics of activation. Most of models of neural stimulation are mathematical extensions of the Hodgkin and Huxley model. Of these, the one that is most accepted and cited for TMS is the Roth peripheral nerve model—a modified active cable type model. This model is similar to peripheral nerve models of electrical stimulation.

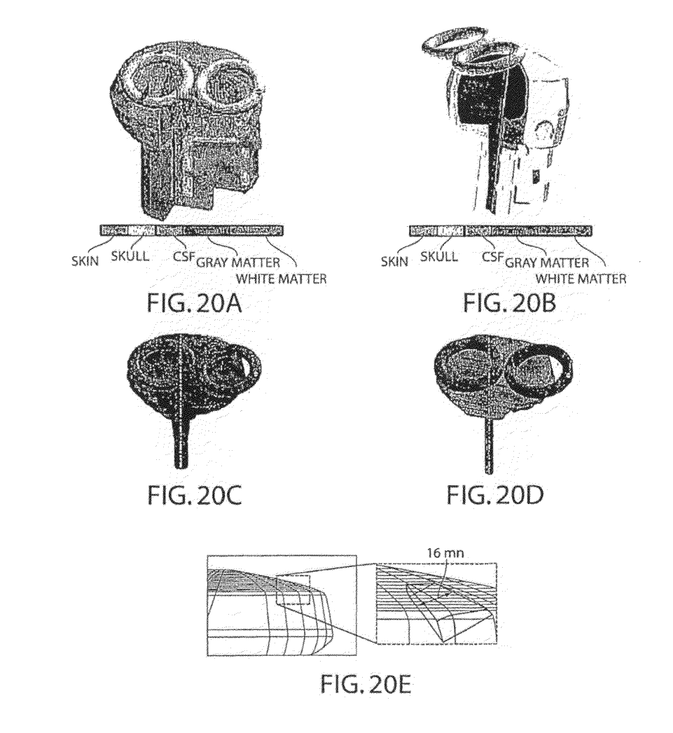

The passive cable model, as illustrated in FIG. 18, is the foundation for the Roth model, is based on classic transmission line theory, where the transmembrane potential, V, can be represented by the following equation:

$${λ}^{2} \frac{{\partial }^{2} V}{\partial {x}^{2}} - V = τ \frac{\partial V}{\partial t} \\ where \\ λ = \sqrt{\frac{{r}_{m}}{{r}_{i}}} , \qquad ( 19 )$$

τ=cₘrₘ, rₘ is the membrane resistance times a unit length, cₘ is a membrane capacitance per unit length, and rᵢ is the axoplasm resistance per unit length. The passive cable model in (19) can be altered by adding an activating function to represent and external current source, as would be seen during electrical stimulation, or the induced electric field, as would be seen during TMS. Here we see the equation altered to include a TMS source:

$${λ}^{2} \frac{{\partial }^{2} V}{\partial {x}^{2}} - V = τ \frac{\partial V}{\partial t} + {λ}^{2} \frac{{\partial }^{2} A}{\partial x \partial t} \\ where \\ - \frac{\partial A}{\partial t} \qquad ( 20 )$$

represents the induced electric field. This is similar to electrical stimulation models that include activating functions. This model predicts the electrotonic conduction along the membrane when the activating function would be below the neural threshold.

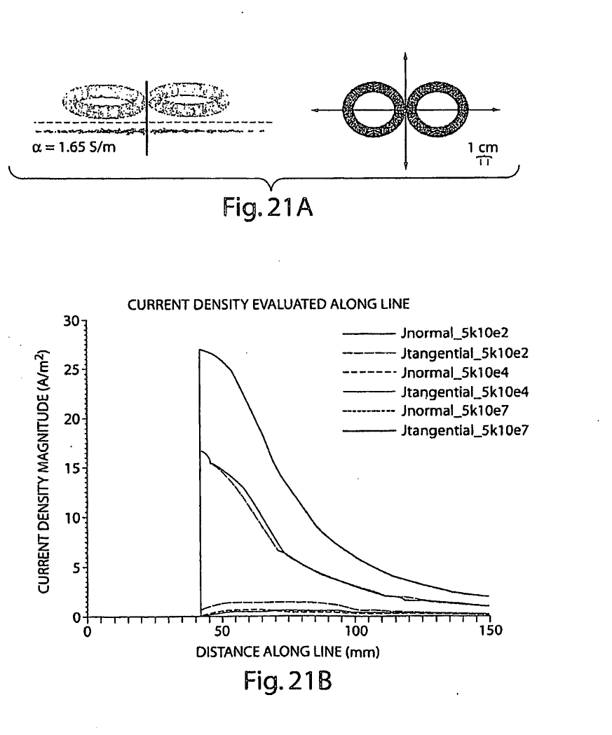

To further increase the detail of the model, one could include the active properties of the axon by implementing the Hodgkin and Huxley model, as illustrated in FIG. 19, to incorporate the neuron's active membrane properties. The Hodgkin and Huxley model includes the voltage/time dependent sodium and potassium channels represented by g_{K} and g_{Na} (conductances per unit area for sodium and potassium), the static leakage channels represented by g_{L}, and the Nernst potential for the sodium, potassium, and leakage ions represented by E_{Na}, E_{K}, and E_{L}. With these additions, the final equation of Roth's model is:

$${λ}^{2} \frac{{\partial }^{2} v}{\partial {x}^{2}} - {g}_{L} ( {E}_{L} - V ) - {g}_{Na} ( {E}_{Na} - V ) - {g}_{K} ( {E}_{Na} - V ) = {C}_{m} \frac{\partial V}{\partial t} + {λ}^{2} \frac{{\partial }^{2} A}{\partial x \partial t} \\ where λ = \sqrt{\frac{1}{2 π {a}^{2} {r}_{i}}} , \qquad ( 21 )$$

a is the axon radius, and Cₘ is given as capacitance per unit area. In the field of electrical stimulation, much work has been done by Rattay and others that shows similar results.

According to the Roth model, the site of neural stimulation (the initiation of action potentials) is found where the spatial derivative of the induced electric field is maximum. One result of this fact is that the coil hot spot of a figure of eight coil does not correspond to the optimal site of peripheral nerve stimulation. The predictions based on the model have been compared to experimental results—agreeing clearly in some cases and less so in others. Finally, it should be clear that this model pertains to long peripheral nerves only and there is no justification to extend this model to cortical neurons. In fact if the same field parameters used for this model were used for cortical neurons the spatial gradients of the electric field would be negligible due to the cortical neurons short length.

Another cable type model which more, accurately depicts cortical neurons is the cable model developed by Nagarajan. It is similar in many respects to Roth's model; yet it begins to account for the smaller size, branching, and terminal endings found in cortical neurons. Nagarajan incorporates boundary type equations into his model, much like what is done for open and closed circuit transmission lines, to model the terminal endings. With this model there are two activation functions, one due to the boundary fields and one due to the induced electric field gradient along the neural fiber axis (as in Roth's model). With this increased complexity, the spatial derivative of the induced electric field is not the primary factor in predicting the activation site as it was in the Roth model, but the field effects at the boundary dominate.

So, in the Nagarajan cable model the excitation site is located at the axon terminals (bouton locations) or at the cell body where the neural axon begins, but no field quantity is solely predictive of the neural site of stimulation or strength of excitation. In the field of electrical stimulation, McIntyre and Grill, Durand, and Rattay have all produced similar models and have found similar results (dependent on the stimulus waveform). The electrical stimulation work of McIntyre and Grill and Rattay is of particular relevance as they have begun to integrate field model solutions with their cable models to give a more complete model of stimulation (and to examine the stimulus waveform's effects on stimulation).

There have been few attempts to explain the biophysical mechanisms of TMS stimulation, however Kamitani et al, have recently generated a model to offer insight into the physiology of stimulation. With a realistic cell model that took into account the dendritic aborization, synaptic inputs, and the various densities of the sodium, potassium (slow and fast channels), and calcium channels they were able to show a few key results; most notably that the induced current within the neurons was directly related to the electric field along the neuron path. Without a synaptic background magnetic stimulation rarely reached threshold, while with a background of synaptic inputs magnetic stimulation brought about burst firing followed by an extended silent period. Bursting was brought about by an influx of Ca²⁺ ions followed by the opening of Ca²⁺ dependent K⁺ channels. Such a result could be the cause for the long-term effects of TMS. With tDCs, there are no relevant biophysical models of altered membrane excitability due to the weak DC currents that the author is aware of.

With both tDCs and TMS, neural studies have been completed to explore the effects of the fields on the cortices and the neural elements. In 1956, Terzuolo et al. studied the effects of DC currents on neural preparations and the relative current to axonal orientation. They found that currents as low as 3.6×10⁻⁸ injected across preparation region could change the frequency of firing, even though they did not directly initiate an action potential. And as mentioned above, in the 1960s Bindman showed that currents as low as 0.25 μA/mm² applied to the exposed pia via surface electrodes (3 μA from 12 mm² saline cup on exposed pia surface) could influence spontaneous activity and the evoked response of neurons for hours after just minutes of stimulation in rat preparations.

And Purpura et al. (1964), showed similar effects in cat preparations for currents as low as 20 μA/mm² from cortical surface wick electrodes ranging in area from 10-20 mm². In 1986, Ueno et al completed work with neural preparations and time changing magnetic fields to ascertain their effects on action potentials, yet flaws with the experimental design limit the relevance of the work as a resistor was impaled into the neural preparation throughout the experiments. In 1990, McCarthy and Hardeem conducted a number of experiments with neural preparations and pulsed toroids, and came to the controversial conclusion that capacitive, not inductive currents, were the cause of magnetic stimulation. They however used sources which were outside of the power spectrum of typical stimulators and thus it is difficult to evaluate their work in terms of clinical TMS stimulation. Unfortunately, these experiments have generated more questions than answers and have done little to shine light on the cellular mechanisms of stimulation.

In terms of the network activity, little is known regarding either technique. Currently only one single network model of TMS exists, but it is hampered by fundamental errors in assumptions on the field dynamics of TMS (for example, it is assumed that the area of stimulation is confined to a cortical region of less than 5 mm in diameter and as has been shown in earlier modeling studies this is not the case [88]). Yet, it is clear from animal experiments that rTMS has an obvious network effect and stimulation effects are physiologically not confined to one brain site [89]. However, no quantitative model has been developed that clearly explains the role that rTMS plays in altering cortical, and thus network, excitability. The network effects of tDCs have been similarly under explored.

Regrettably, there is no clear understanding of the true biophysical electromechanics of TMS or tDCs stimulation. With TMS the models that exist clearly bring up many relevant issues, but unfortunately they have not been tested on a cellular level due to the technical difficulties associated with the process. Until a methodology, which will not be corrupted by the field artifact, is implemented then analogies between microstimulation and TMS will be all that researchers have to rely on. With tDCs, little work has been done to ascertain the cellular effects of the weak currents. This is unfortunate because the technological hurdles that exist with TMS are not present with tDCS. Hopefully future work will shed more light on both processes.

Both TMS and tDCs share many similar uses as both modalities allow investigators to modulate the level of cortical excitability, however TMS has an additional utility in that it can be used to actively stimulate neurons and thus map cortical function and indirectly assess the level of cortical excitability by means of evoked potentials. Below, the varied uses will be explained in separate sections; including sections on TMS as tool for cognitive neuroscience research, TMS as a clinical diagnostic tool, and detailing tDCs and TMS for the treatment of neuropathologies. Particular detail will be provided in explaining the use of TMS in assessing the level of cortical excitability by presenting an entire cognitive experiment because many of the topics presented in the section will be revisited throughout the thesis. The other topics will only be presented superficially as many reviews and textbooks exist that comprehensively cover the information.

As a research tool, TMS can be used for brain mapping to establish the link between cortical location and function, and thus augment conventional neuroimaging techniques. This work is similar to the work done by Wilder Penfield in the 1940's where he mapped out functional regions of the brain during epileptic surgery by stimulating the exposed cortex directly with an electrical source, but now with TMS functional regions can be mapped out without the need of surgery and direct cortical contact.

More recently, TMS has also found an increasing use in the study of higher cognitive functions including the study of language, visual processing, and memory. For instance in the study of language, it has been shown that TMS can induce anarthria, speech arrest, in patients; in the future this could be a valuable tool in determining the language dominant hemisphere prior to brain surgery. Repetitive TMS has been shown capable of modulating the levels of cortical excitability at times beyond the duration of rTMS itself, depending on the intensity, frequency, and duration of stimulation oftentimes the after effects are termed “reversible lesions”. Taking advantage of the lasting effect of rTMS, Kosslyn et al demonstrated the requirement of area 17 during depictive visual imagery. In terms of memory processing, Grafman et al, used the after effects of rTMS to modulate subject performance in a simple recall task and showed the potential of rTMS as a tool to study memory function. Below, a study focused on assessing alterations in cortical excitability brought on via subconscious processing of self awareness is presented and reviewed.

In this study, the unconscious modulation of corticospinal excitability was probed with transcranial magnetic stimulation (TMS). In previous experiments in our laboratory, it was shown that the presentation of pictures containing elements of one's own face can alter the level of motor cortex excitability [100]. Motor evoked potentials (MEP) from the first dorsal interosseus (FDI) muscle were increased when TMS was applied to the right motor cortex during presentation of a picture that contained elements of the subject's face. Modulation of MEP magnitudes have been hypothesized to reflect the hemispheric level of cortical excitability. And thus, TMS provides researchers with a tool to gauge the level of cortical excitability during stimulation.

This task makes use of a technique known as backwards masking. Essentially, when a visual image of short duration (target, shown for 30 ms) is immediately followed by the presentation of a second visual stimulus (mask, shown for 90 ms), conscious awareness of the target image can be prevented.

Fifteen right-handed, healthy subjects participated in this study, which was approved by the local institutional review board. Written informed consent was obtained from all subjects. Handedness was defined by the Edinburgh Inventory. Participants were randomly assigned to one of two conditions: (i) covert, in which the subject's face was masked (8 subjects); (ii) overt, in which the subject's face was not masked (7 subjects).

Subjects viewed a sequence of 192 pairs of greyscale images containing a target face (30 ms) immediately followed by a mask (60 ms), presented on a computer screen at a distance of 100 cm. A fixation point was shown for 1000 ms, followed by the target and mask stimuli.

A blank screen followed the mask and the next trial was initiated after the participants had verbally classified the face they had perceived (i.e. the mask) as belonging to either group 1 or 2, according to a chart they had previously studied. The target image consisted of the subject's own face (self) on half of the trials. The remaining half consisted of pictures of unknown individuals (others), which were also used as masks (four different pictures randomly presented). A picture of the subjects' face was obtained prior to the experiment under the pretense that it would be used in the image set of other participants. In the overt condition, the target and mask images were switched, such that all target images were of unknown faces while half of the masks consisted of images of the subjects' own face. In this experiment, subjects had to detect the presence of a small triangle on the perceived image. After completion of all stimulus presentations, they were asked to report their subjective feelings about the stimuli and were explicitely asked whether they had seen parts of their own face at any point during the experiment.

During each stimulus presentation, a single TMS pulse was applied to the right or left motor cortex at various intervals (100, 200 and 400 ms) following presentation of the target picture. TMS was delivered with a commercially available 70 mm figure-of-eight coil and a Magstim 200 transcranial magnetic stimulator (Magstim Company, Dyfed, UK). The stimulation coil was placed flat on the subjects' scalps over the optimal position for induction of MEPs of maximal peak-to-peak amplitude in the contralateral target muscle (first dorsal interosseus muscle; FDI). This optimal scalp position was defined following recommended guidelines in each study subject at the beginning of the experiment.

Two surface electrodes were placed on the belly and tendon of the right and left FDI to collect MEPs. The EMG signal was amplified (to 1.0 mV) and filtered (band pass 20-1000 Hz) using a Dantec counterpoint electromyograph (Dantec, Skovlunde, Denmark). The signal was digitized using PowerLab 16S (AD Instruments Limited, Hastings, UK) with a sampling rate of 2 kHz and stored on a computer for off-line analysis. The collected MEPs were rectified and the area under the curve was calculated. TMS intensity was set at approximately 110% of motor threshold (MT), which was defined as the minimal intensity of stimulation capable of inducing MEPs >50 μV peak to peak amplitude in ≧6/10 consecutive trials.

Sixteen MEPs were collected for each time interval (100, 200 and 400 ms), hemisphere (left and right), and condition (self and other) for a total of 192 MEPs. The left and right hemispheres were stimulated separately with the order counterbalanced across subjects. Intervals and conditions were varied randomly. In order to minimize the inherent variability of TMS-induced motor evoked potentials, especially across hemispheres, raw MEP values were transformed into a “self” ratio (self/other*100). The effect of masked pictures on corticospinal excitability was evaluated with a two-way repeated measures analysis of variance (ANOVA), with ‘INTERVAL’ (100 ms vs 200 ms vs 400 ms) and ‘HEMISPHERE’ (left vs right) as factors.

In the covert condition, subjects correctly identified the mask in 76% of the trials. There was no difference in performance whether the subject's face or a stranger's face was masked (Self: 80%, Other: 79%; t₇=0.54, P=0.60). In the overt condition, subjects' performance at detecting the triangle was 100%. None of the subjects reported seeing any part of their own face in the covert condition. Furthermore, when three subjects were explicitely instructed to detect the presence of a masked self image in the same paradigm used in the main experiment, performance was near chance (51%±8). We can therefore conclude that the masking paradigm prevented conscious perception of the target faces.

In the covert condition, mean MT was 50.0% of maximum stimulator output in the right hemisphere and 49.9% in the left hemisphere. There was no significant difference between the two values (t₇=0.74, P=0.94). Stimulation intensity was set at an average of 57% and 58% for the right and left hemispheres, respectively (non-significant difference; t₇=−0.57, P=0.59). In the overt condition, mean MTs were 52.0% and 53.4% for the right and left hemispheres, respectively (N.S.; t₆=−1.2, P=0.28). Stimulation intensity was set at 60.3% and 62.7% for the right and left hemispheres, respectively (N.S.; t₆=−0.96; P=0.37).

In the covert condition, a repeated measures ANOVA revealed a significant main effect of HEMISPHERE [df (1.14); F=7.13; P=0.01; FIG. 2]. In 7 of the 8 subjects, the self ratio was higher in the right than in the left hemisphere, and was above 100. This indicates that MEPs were modulated by the unconsious processing of a self-image. When all intervals were collapsed, the mean self ratio was 112.04 for the right hemisphere and 95.44 for the left hemisphere. There was no significant effect of INTERVAL (P=0.36) and the interaction was also non-significant (P=0.82). No significant effects were detected for the overt condition.

A discussion of self awareness and the cognitive implications of this study will not be discussed here as they are outside the scope of this application. But rather, the excerpts from the publication were included to depict how TMS was implemented as a tool to assess the level of cortical excitability. Note that level of cortical excitability was determined by comparing the strength of the neural response, indirectly measured via EMG recordings during activation of specific muscles, to the level of the machine output—which reflects the power output and the strength of the magnetic field for a given machine. Thus within subjects, TMS provides a means to assess the relative level of cortical excitability (note that there is a large variability in MEP thresholds across subjects). This technique is the standard in TMS based cognitive neuroscience experiments. In future parts of this thesis, we will come back to this concept of cortical excitability.

TMS also has a great number of diagnostic uses, related to the analysis of TMS evoked potentials. It can be used in the evaluation of various neuropathologies (e.g. multiple sclerosis, amyotrophic lateral sclerosis, and spinal cord injury). For example in such cases, Motor Conduction Times (MCT) measurements could be made to test the integrity of the neural pathways under examination. Delayed conduction times would be expected in the presence of numerous pathologies, and have been shown conclusively in many cases. Additionally, certain pathologies (related to various motor disorders) have characteristic MEP shapes, and a trained physician can thus use TMS to diagnose certain disorders.

Both rTMS and tDCs offer clinicians the ability to modulate the level of cortical excitability. As explained above, with rTMS one can alter the level of cortical excitability past the duration of stimulation using 1 Hz TMS stimulation to inhibit and 20 Hz TMS stimulation to excite the cortical tissue. With tDCs, one has the same ability to raise and lower the level of cortical excitability based on the current polarity (and the location of the scalp electrodes). Thus, neuropathologies that are characterized by abnormal levels of cortical excitability make candidates for tDCS or TMS treatment. For instance following a stroke, it is thought that there is an imbalance in the level of excitability between the two hemispheres brought about by the ischemic event. In theory, one hemisphere is hyper-excited and the other over-inhibited due to a released intra-cortical inhibition. Thus, with either technique one could theoretically bring the hemispheres back into balance by raising or lowering excitability of the effected hemispheres.

The remainder of this application will continue to explore TMS and tDCs. It is divided into three sections. Section one will present a healthy head model of TMS and an experiment quantifying the dielectric response of the tissues exposed to stimulation as foundations for the rest of the manuscript. Section two will focus on TMS and neuropathology, covering stroke and atrophy from a theoretical viewpoint and assess the use of TMS for neurorehabilitation via clinical experiments. Section Three will focus on tDCs, repeating the analysis that was completed for TMS (both for the healthy human head and the pathology cases). Finally, the application will compare the modalities.

Section 1

The two experiments covered in this section focus on TMS in the healthy human head to provide a basic understanding on which the remainder of the TMS experiments will be based. The two experiments entail an FEM modeling study of a healthy human head and an impedance analysis of the head tissues measured in-vivo in an animal model. The electromagnetic and electrophysiological principles that are addressed in the FEM healthy head model serve as a conceptual foundation for the remainder of the thesis and serve as a comparative basis for the pathologic situations addressed later. The impedance analysis will be used to evaluate the electromagnetic models included in this application.

Despite the widespread use of TMS, much is still to be learned about the underlying electromagnetic field distributions, and particularly the induced current density. Phantom, animal, and in vivo human studies have been conducted to explore the induced current distributions. These studies have all provided important information; however, current technical limitations preclude the complete characterization of the electromagnetic field distributions via this type of experimentation and necessitate the development of theoretical studies. Numerous theoretical models have been developed to provide scientists and physicians with a view of the electromagnetic field distributions generated in biological tissue during TMS. Nevertheless, many of the commonly accepted results of earlier field models need further evaluation and issues relating to the electrical properties of biological tissue have just begun to be explored.

The majority of models to date have all been based on “infinite half-planes” and perfect spheres. One of the implicit results of these models is the absence of electric field components normal to the cortical tissue interface. From this, many researchers have drawn the conclusion that fields normal to the cortex will be minimized in the human head and neurons are preferentially stimulated which run parallel to the cortical tissue interface. However, this prediction is the result of the simplified symmetrical geometries used in these models. For example, Branston and Tofts proved the absence of electric fields normal to the bounding surface of both an “infinite half-plane” and spherical conductor with limited current sources (ramp shaped). Heller and Hulsteyn later extended their argument to include all current source types of TMS. Cohen and Cuffin came to this same conclusion by applying the reciprocity theorem to the results of earlier MEG studies. Yet we know of no published accounts that test for the presence of currents normal to the cortex in more realistic head geometries; thus, this unproven assumption is still pervasive in TMS research.

Another concept that requires further exploration is that tissue boundary layers surrounding the cortex have a limited influence on the final stimulating field. Although this assumption has merit for simplified symmetrical models, it has become evident that removing or ignoring layers of the system will provide incomplete or inaccurate results and that tissue changes in the system will alter the induced fields. For instance, Scivill, Barker, and Freeston produced a finite element model of spinal cord stimulation that took into account the tissue asymmetries and provided a more complete model that included the cerebral spinal fluid (CSF), which had been earlier ignored. The CSF, which has a conductivity that is approximately 4 to 5 times higher than the surrounding tissues, was shown to effectively shunt the induced currents away from the spinal cord. Ueno and Liu generated a model that accounts for changes in conductivity and the nature of tissue inhomogenities.

Using a square model with two dissimilar conductors they showed that the conductive boundaries have an altering effect on the induced field distributions. Similarly, Miranda et. al. generated a three sphere model that clearly showed that both heterogeneities and anisotropies clearly influence the final TMS fields. Even with these important results we are unaware of any studies that explore this effect in more realistic geometries with multi-tissue systems. It is also clear that both the conductivity and permittivity of the tissues in the head are dispersive, yet no work has been published on the dispersive effects of biological tissues on TMS.

Taking these issues into account, the initial goals of this study were to develop a more realistic TMS head model that would allow: a) the investigation of tissue boundary effects on the induced current densities; b) an analysis of the symmetry conclusions drawn from previous studies specifically testing for the presence of radial currents; c) the investigation of how local perturbations in the tissue geometry can alter the resulting current distributions in a multi-tissue head geometry; and d) an account of the dispersive properties of tissue and a means to test how changes in tissue permittivity can affect the induced current distribution.

A new sinusoidal steady state finite element model (FEM) was developed using the Ansoft 3D Field Simulator software package with the eddy current solver. Initially an MRI guided three-dimensional CAD rendering of the human head was generated using the Ansoft package. The MRI (Siemens Magneton Vision 1.5 T scanner saved in Analyze format: 256×256×160, 1 mm³ voxel size) of a thirty-eight year male with no neurological abnormalities was used to guide the generation of the Ansoft Macro code for the initial CAD model geometry. The model was generated to include the skin, skull, cerebral spinal fluid (CSF), gray matter, and white matter. The average thickness of the tissues varied considerably with anatomical position but in the tested regions proximal to the stimulation source they roughly followed a 4-5 mm skin thickness, a 5-10 mm skull thickness, a 2-3 mm CSF thickness, a 4-5 mm gray matter thickness, and a 130 mm distance from ear to ear in the white matter.

The tissue conductivities, while somewhat attenuated below 1 kHz stimulation frequencies, were considered essentially constant for the frequencies of TMS (the power spectrum of typical stimulators is composed of components less than 10 kHz) and assigned the mean value from multiple references; skin at 0.465 S/m, bone at 0.010 S/m, CSF at 1.654 S/m, gray matter at 0.276 S/m, and white matter at 0.126 S/m. The actual low frequency permittivity value of biological tissues is still an area of ongoing research; however, it is believed that alpha dispersion occurs in biological tissues within the frequency range of TMS. Many researchers have experimentally addressed the issue and others have developed models of the process and reviewed dispersion in biological material.

While the trend of increasing permittivity with decreased frequency is seen throughout the literature there is a limited consensus on the value of the relative permittivity magnitudes in biological tissues where values in excess of 10⁷ and as low as 10² have been reported in the TMS frequency spectrum. Thus, the tissue permittivity values were set as variables and we tested values of the magnitude of 10² εₒ, 10⁴ εₒ, and 10⁷ εₒ with differences between the tissues following the trends in the literature, primarily theorized to be caused by cellular organization and counterion diffusion effects, as shown by the mean conductivity values and relative permittivity values in Table 2 below.

TABLE 2

| | | Relative | Relative Permittivity | Relative Permittivity |
|---|---|---|---|---|
| | Mean | Permittivity Scheme | Scheme | Scheme |
| Tissue | Conductivity (S/m) | 10² εₒ (F/m) | 10⁴ εₒ (F/m) | 10⁷ εₒ (F/m) |
| Skin-Scalp | 0.465 | 1.2 × 10² εₒ | 1.2 × 10⁴ εₒ | 1.2 × 10⁷ εₒ |
| Bone-skull | 0.010 | 0.8 × 10² εₒ | 0.8 × 10⁴ εₒ | 0.8 × 10⁷ εₒ |
| Cerebral | 1.654 | 0.60 × 10² εₒ | 0.60 × 10⁴ εₒ | 0.60 × 10⁷ εₒ |
| Spinal Fluid | | | | |
| Gray Matter | 0.276 | 1.2 × 10² εₒ | 1.2 × 10⁴ εₒ | 1.2 × 10⁷ εₒ |
| White Matter | 0.126 | 1.2 × 10² εₒ | 1.2 × 10⁴ εₒ | 1.2 × 10⁷ εₒ |

As stated, there is still considerable debate about the low frequency permittivity values of the tissues; thus, this author chose to allow for the inclusion of the high values as predicted by existing alpha dispersion theories. If the extreme values of current alpha dispersion theories are valid, σ can be much less than ωε or on the same order of magnitude at low frequencies; or equivalently the charge relaxation times of the tissue (relaxation time=ε/σ) can be of the same order of magnitude or greater than the time scale of the stimulating current source. For instance, if the relative permittivity of the gray matter is 10⁷ and the conductivity is 0.276 S/m then its charge relaxation time is approximately 0.32 ms, which is comparable in magnitude to the time scale of the source terms above 1 kHz. Thus, the displacement currents cannot be ignored when evaluating these situations and one would have to solve:

The Ansoft package numerically solves the problem via a modified T-Ω method. The solution process was set with a Neumann boundary condition at the edge of the problem region. The source was modeled as a figure of eight coil with two 3.5 cm radius windings made of a single turn of 7 mm radius copper wire. The current level and frequency of stimulation were set as variables to allow for future reconstruction of the transient solution via Fourier analysis and the testing of tissue dispersive effects. The copper was modeled as a perfect conductor (currents constrained to the surface) with the permittivity set to εₒ, conductivity of 5.8×10⁷ S/m, and a magnetic permeability of 1μₒ.

The source coil was located approximately over the motor cortex and tangential to the scalp interface with the inclusion of an insulating layer (a 7 mm free space gap was included between the tissue and copper core to account for the insulating layer found in commercially available coils). Solutions were obtained for the source set at 5 kHz with a 1.8×10³ A peak current (5.65×10⁷ A/s, rate of change of the peak current with time) and for the source set at 100 Hz with a 9×10⁴ A peak current (5.65×10⁷ A/s). The 5 kHz frequency component of the source was chosen based on the Magstim Rapid Stimulator.

This device is commonly used in the medical environment and has a peak frequency component at approximately 5 kHz in its power spectrum. The 100 Hz component was included for a comparison of the dispersive effects between frequencies, as the alpha dispersion effects are more pronounced at lower frequencies (i.e., the permittivity values are inversely related to frequency). The model solutions will be referred to by both their source frequency and permittivity magnitude. Thus, the 100 Hz10²εₒ solution would refer to the solution with a 100 Hz source frequency and a 10² relative permittivity magnitude.

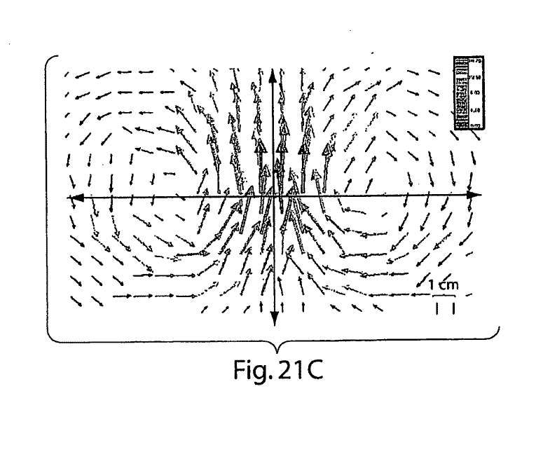

A model was also constructed to make a preliminary investigation of how local modifications of the cortex can perturb the induced current densities. A 22 mm long 18 mm wide cut in the cortex was replaced with CSF and placed at the location of the expected coil hot spot as shown in FIG. 20, plot E. This model was solved with 10², 10⁴, and 10⁷ tissue permittivity values and with a 5 kHz 1800 A peak current source (5.65×10⁷ A/s). Because this change from the initial model geometry could represent an infarction site resulting from a stroke or other pathologies, we will refer to this as the pathological model in the following discussion (i.e., the Pathological 10²εₒ solution will refer to this model solved for the 10²εₒ permittivity level).

To assess the validity of our solutions we also constructed a model where the cortical cut was made in the opposite hemisphere (i.e., on the opposite side of where the stimulating coil and the cortical hot spot were located). We will refer to this as the mirror model, as the cortical cut was made in the reflected hemisphere, and use the same naming convention as earlier (i.e., Mirror 10²εₒ will refer to this model solved for the 10²εₒ permittivity levels).

During the simulations the jawbone and nasal bone were excluded from the solution region for computational efficiency. The magnitude of the magnetic field was substantially attenuated at their locations and their exclusion had negligible effects on the solution (based on preliminary analytical solutions and numerical simulations). Had we been modeling stimulation in the inferior area of the frontal lobe, this simplification would not have been merited and the details could not be excluded. We will explore this further in the results and discussion. The problem region for the head model was defined as a 1953×1982×3031 mm box where the head took up less than 5% of the total space (with a free space background; i.e., permittivity of free space, permeability of free space, and a conductivity of 0 S/m).

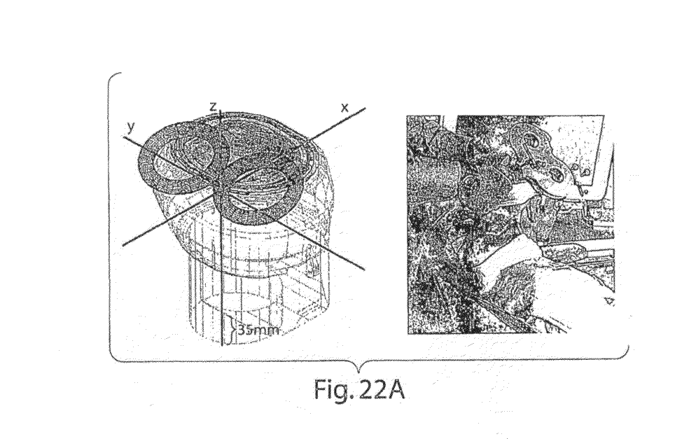

A single conductor “infinite half-plane” study was also completed to further assess the validity of the solution process. A geometry was constructed with a large square box (1 m³) of conductive material (1.65 S/m) and tested with 5 kHz source at 1.8×10³ A peak current (5.65×10⁷ A/s). A figure of eight coil (3.5 cm radius and 7 mm copper core radius) was positioned 10 mm above the conductive interface, directly in the center of the box face, and rotated at a 20 degree angle to the interface, such that the source current had x, y, and z components as shown in FIG. 21, plot A. The relative permittivity of the conductive system was tested for values of 10² εₒ, 10⁴ εₒ, and 10⁷εₒ.

The Ansoft FEM solver was set to follow an adaptive iterative process with the convergence limits determined by the energy error in the system. The Ansoft package defines the error of the individual tetrahedron as ∇·B and the total time averaged magnetic field energy for the system as

$$\frac{1}{4} \int \hat{B} \cdot \hat{{H}^{\hat{}}} \partial V .$$

During each solution pass the solver calculates the total time average energy of the system, the total error of the system, and the energy error of the system based on the energy of the B field components where ∇·B≠0 (reported as a percentage of the total energy). Following each pass, the solution is adapted automatically by refining the mesh and adjusting the individual tetrahedra with the largest energy error—this is done by replacing the tetrahedra with the largest error with multiple smaller ones or by adjusting the size of the tetrahedra in the area of the error. The criterion for model convergence was defined as an energy error below 2.5% and every model tested converged by this definition. Where the results are reported as magnitudes, they indicate the magnitude of the sinusoidal steady state current density in the units of A/m², unless otherwise noted.

Where the results are reported at phase, the phase angle will be explicitly given (i.e., since analyses were performed in the sinusoidal steady state, we explicitly define the phase angle). However, we do not in general explore the phase differences between solutions here, as they have no direct bearing on the reported results. But, they would be necessary in order to reconstruct a transient result, the details of which are addressed in the discussion.

One of the results of the “infinite half-plane” model that was discussed in the first section is the absence of currents normal to the interface at any point within the volume regardless of the geometry or orientation of the stimulating coil. This is a general analytical result for symmetrical structures and was one of the initial tests used to assess the validity of the modeling process at hand. To verify this result we calculated the magnitude of the current density components both tangential and normal to the planar interface. The 10²εₒ and 10⁴εₒ solutions converged to approximately the same result (i.e., the results that are reported here differed by less than +/−2.5% from solution to solution). For all of the solutions (10²-10⁷εₒ), the magnitude of tangential current density components was substantially larger than the normal component.

In FIG. 21, plot B, the tangential and normal current density components are plotted along a line perpendicular to the conductive interface placed through the figure-of-eight coil's center and through the point of the maximum normal current component in the half plane, the location of which can be seen in FIG. 21, plot A. The maximum tangential current density magnitude was 16.7 A/m² for the 10²-10⁴εₒ solutions and 27.1 A/m² for the 10⁷εₒ solution. The maximum normal current density component was 0.42 A/m² for the 10²-10⁴εₒ solutions and 1.2 A/m² for the 10⁷εₒ solution. In FIG. 21, plot C, the current density vectors for the 10⁴εₒ solution are plotted at phase, with the phase angle set to 0 degrees, along a transverse plane 1.0 cm from the interface (the plane is shown in FIG. 21, plot A).

In this model the symmetry effects were evident as predicted by other authors and the permittivity effects only became evident for the 10⁷εₒ solution, which can be predicted based on the charge relaxation time of the material. Even when the displacement current reached non-negligible levels, the symmetry conditions still dominated and the magnitude of the tangential components was much larger than the normal component of the induced current density. However, the magnitude of the current density nearly doubled in this case compared to the others and this correlated well with the increase in the magnitude of the complex conductivity of the material (here the complex conductivity is defined as σ*=σ+jωε).

Although many conditions could be tested with this model, one of the main goals was to evaluate the induced current distributions in the tissue and the way in which the anatomical asymmetries and differing tissues affect the currents. The 100 Hz10²εₒ, 100 Hz10⁴εₒ, 5 kHz10²εₒ, and 5 kHz10⁴εₒ solutions converged to the same result (i.e., the results that are reported here differed by less than +/−2.5% from solution to solution). For all of the solutions except the 5 kHz10⁷εₒ solution the maximum current density was found in the CSF, the tissue layer with the largest conductivity.

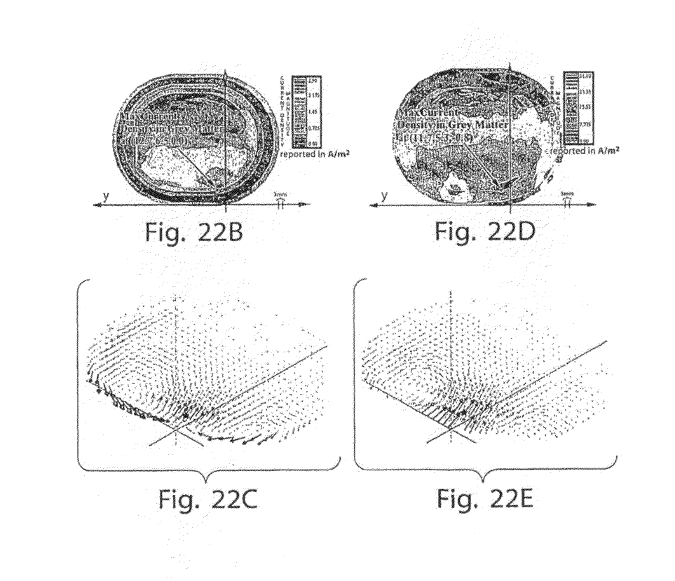

For the 5 kHz10⁷εₒ solution the maximum current density was found in the skin, the tissue with the largest magnitude of complex conductivity at 5 kHz. The maximum current density was the 17.5 A/m² centered at (8.7,4.2,0.8) for the 100 Hz10²εₒ, 100 Hz10⁴ε_{O}, 5 kHz10²εₒ, and 5 kHz10⁴εₒ solutions, 17.7 A/m² centered at (9.6,4.9,1) for the 100 Hz10⁷εₒ solution, and 39.5 A/m² centered at (5.7,6.0,4.0) for the 5 kHz10⁷εₒ solution (the coordinate system is defined in FIG. 22, plot A in 3-dimensions and uses the units of millimeters. The coordinate system is further highlighted in 2-dimensions in FIG. 22, plot B and D.).

For every solution the maximum current density in the gray matter was found along the CSF/gray matter interface. The maximum cortical current density was 2.9 A/m² centered at (12.7,6.5,0) for the 100 Hz10²εₒ, 100 Hz10⁴εₒ, 5 kHz10²εₒ, and 5 kHz10⁴εₒ solutions, 3.05 A/m² and centered at (12.5,6.4,−0.1) for the 100 Hz10⁷εₒ solution, and 31.1 A/m² centered at (11.7,5.3,−0.8) for the 5 kHz10⁷εₒ solution (FIG. 22, plot B and D). Note that the approximate 10-fold increase in cortical current density for the 5 kHz10⁷εₒ solution correlated to the approximate 10-fold increase in the magnitude of the complex conductivity of the tissue. The reported values are tabulated in Table 3 below.

TABLE 3

| | Passes to | | Energy Error |
|---|---|---|---|
| Model | Convergence | Tetrahedra | (%) |
| 100 Hz10² | 6 | 148473 | 0.86 |
| 100 Hz10⁴ | 6 | 131204 | 1.31 |
| 100 Hz10⁷ | 6 | 148370 | 0.58 |
| 5 kHz10² | 6 | 168140 | 1.40 |
| 5 kHz10⁴ | 6 | 148824 | 0.97 |
| 5 kHz10⁷ | 7 | 156720 | 0.21 |
| Pathological 10² | 6 | 170115 | 1.58 |
| Pathological 10⁴ | 6 | 170303 | 1.67 |
| Pathological 10⁷ | 6 | 170061 | 1.29 |

The magnitude and vector orientation of the current density was analyzed throughout the model in transverse, sagital, and coronal planes. The current density magnitude is plotted in FIG. 22, plot B and D on the plane of maximal cortical current density for the 5 kHz10⁴εₒ and 5 kHz10⁷ εₒ solutions. To highlight the magnitude of the cortical current in these figures, the color scale maximum is set at the maximum of the cortical level for each case, the currents in the more superficial layers that exceed this value are cut off at the maximum. For each of the solutions the vector orientation showed the greatest variation along the transverse planes following a figure-eight path with the greatest irregularity at the tissue boundaries.

FIG. 22, plot C and E show vector plots of the current densities on the identical transverse slices for the 5 kHz10⁴εₒ and 5 kHz10⁷εₒ solutions. The vector plots graphically indicate the magnitude of the current density vectors through both the size and color of the vector arrows and were generated at phase with the phase angle set to 0 degrees. For every solution, current density components were found normal to the CSF/gray matter interface, the relative values of which will be addressed in the next section.

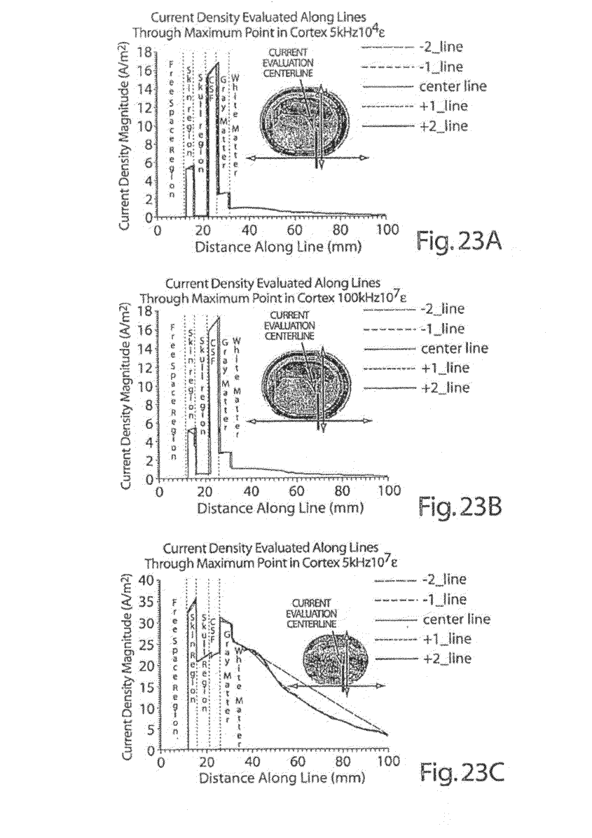

Stair step jumps in the current density magnitude were seen at the tissue boundary interfaces in every solution. In the 100 Hz10²εₒ-5 kHz10⁴εₒ solutions the jumps in current density correlated to the conductive changes in the tissue, as illustrated in FIG. 23, plots A and B. However, in the 5 kHz10⁷εₒ solution the stair step jumps were less clear and appeared to be primarily influenced by the differences in the tissue permittivity and secondarily by the conductivity when the permittivities were equal (as can be seen at the gray matter/white matter interface), as illustrated in FIG. 23, plot C. The current density magnitudes were slightly higher in each of the tissues for the 100 Hz10⁷εₒ solution compared to the 100 Hz10²εₒ, 100 Hz10⁴εₒ, 5 kHz10²εₒ, and 5 kHz10⁴εₒ solutions; but the overall contribution from the displacement current was minimal except in the skull where the current density magnitude jumped from a maximum of 0.15 A/m² to 0.47 A/m² (see FIG. 23, plot B). The 5 kHz10⁷εₒ solution varied considerably from the other results and the current density magnitude was higher in all of the tissues in this model due to the contribution from the displacement current.

For each of the individual solutions, the current densities showed a consistent behavior around the cortical hot spot. In FIG. 23, plot A, the current density magnitudes are shown as evaluated along parallel lines that penetrate the model through the location of maximum cortical current density and pass through all the included tissues for the 5 kHz10⁴εₒ solution. The lines were placed to intersect and surround the maximum cortical current density location in the solution (every +/−1 mm on the y-axis for 5 mm). The location of the center evaluation line, shown in FIG. 23, plot A, is located in the transverse plane where the maximum cortical current density is found. It runs through the point of maximum current density in the cortex on a path parallel to the x-axis in the figure (from (−13,6.5,0) to (87,6.5,0) according to the coordinate system defined in FIG. 22, plot A).

The other evaluation lines were placed in parallel to the center-most-line every +/−1 mm along the y-axis. In FIG. 23, plot B, the current density magnitudes are shown as evaluated along the lines for the 100 Hz10⁷εₒ solution. The evaluation lines are shifted −0.1 mm on the y axis and −0.1 mm on the z axis to account for the altered location of the maximum cortical current density. In FIG. 23, plot C, the current density magnitudes are shown as evaluated along the lines for the 5 kHz10⁷εₒ solution. The location of the evaluation lines is shifted −1.2 nun on the y axis and −0.8 mm on the z axis to account for the altered location of the maximum cortical current density. The same consistency was seen along the z-axis for approximately 4 mm in each case.

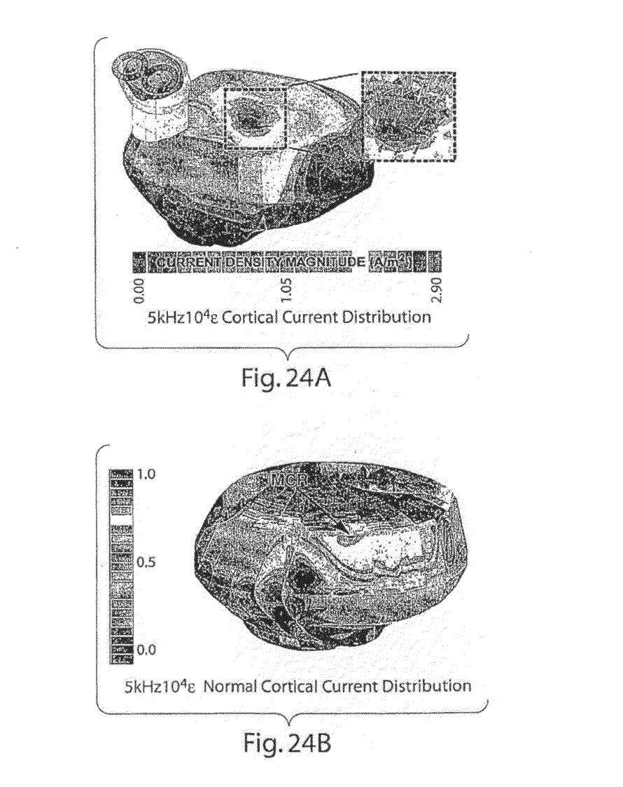

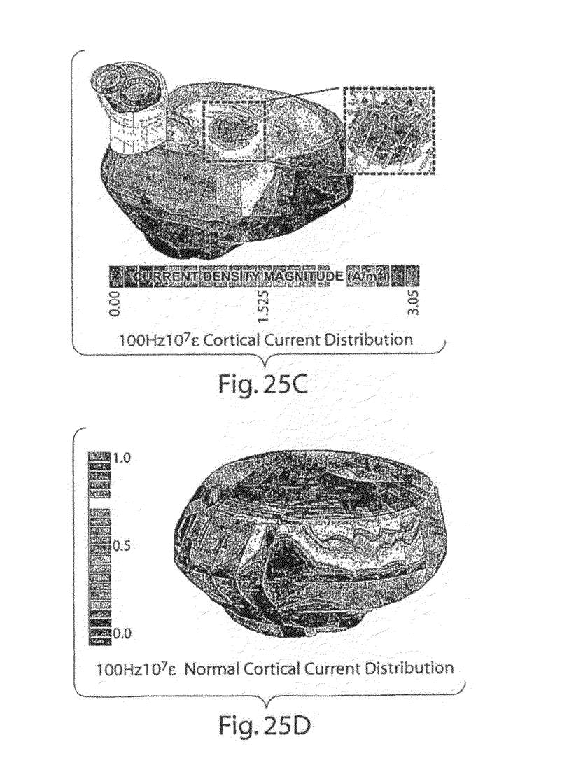

Current density components normal to the CSF/gray matter interface were found in every solution evaluated (see FIG. 24, plot A and FIG. 25, plot C for the examples of the 5 kHz10⁴εₒ and 100 Hz10⁷εₒ solutions). The relative magnitude of the current density component normal to the cortical interface was calculated by taking the dot product of the surface normal with current density along the gray matter interface and dividing this result by the current density magnitude. The results are reported as a ratio of the magnitude of normal current density to the magnitude of the current density, with a maximum magnitude of 0.72 for the 100 Hz10²εₒ, 100 Hz10⁴εₒ, 5 kHz10²εₒ, and 5 kHz10⁴εₒ solutions (at a location where the current density magnitude was 1.57 A/cm², a value of 0.54 of the maximum cortical current density), 0.65 for the 100 Hz10⁷εₒ solution (at a location where the current density magnitude ranged from approximately 1.43 A/cm² to 1.56 A/cm², a value of 0.46 to 0.51 of the maximum cortical current density), and 0.63 for the 5 kHz10⁷εₒ solution (at a location where the current density magnitude ranged from approximately 14.0 A/cm² to 15.6 A/cm², a value of 0.45 to 0.50 of the maximum cortical current density).

The 100 Hz10²εₒ, 100 Hz10⁴εₒ, 5 kHz10²εₒ, and 5 kHz10⁴εₒ maximum ratio was localized to an area of approximately 46.8 mm² at (3.6,21.6,−9.3), 19.9 mm away from the cortical hot spot. In the region of maximum cortical current density the maximum value of the ratio was approximately 0.31. The maximum ratio area was less localized and smaller in magnitude on the gray matter surface for both the 100 Hz10⁷εₒ and 5 kHz10⁷εₒ solutions. In the region of maximum cortical current density, the maximum value of the ratio was approximately 0.28 for both of the solutions. FIG. 24, plot B and FIG. 25, plot D depict the current density magnitude and vector plot in magnified view on the surface of the gray matter for the 5 kHz10⁴εₒ and 100 Hz10⁷εₒ solutions. FIG. 24, plot B and FIG. 25, plot D plot the ratio on the surface of the gray matter for the 5 kHz10⁴εₒ and 100 Hz10⁷εₒ solutions (the 5 kHz10⁷εₒ solution was very similar to the 100 Hz10⁷εₒ solution).

The induced current densities were evaluated for the pathological model solutions. The Pathological 10²εₒ and Pathological 10⁴εₒ solutions converged to approximately the same result. For these solutions, the maximum current density induced in the tissue was still in the CSF, but it was found to be 1.3 A/m² higher and 14.9 mm away from where it was found in the analogous non-pathological cases. It was centered at (22.8,6.4,5.2) with a magnitude of 18.8 A/m². In the Pathological 10⁷εₒ solution, the maximum current density induced in the tissue was found in the skin, centered at (5.5,5.7,4.1), with a value of 39.5 A/m². This was of the same magnitude and 0.4 mm away from where it was found in the 5 kHz10⁷εₒ solution. In the region of the cortical hot spot, the Mirror pathological models converged to results analogous to the equivalent non-pathological models (i.e., those without cortical modifications). We will not discuss them further here, except to say that cortical modifications outside of the region of the coil focus did not significantly alter the currents in the region of maximum cortical current density in our model (i.e. the above results were not a mathematical anomaly caused by randomly altering the model geometry).

In each of the pathological solutions, the maximum cortical current density was still found at the CSF/gray matter interface but the location and magnitude changed in each case compared to the analogous non-pathological solutions. The magnitude increased and the position was displaced towards the edge of the cortical cut. In the Pathological 10²εₒ and Pathological 10⁴εₒ solutions, the cortical current density was 1.1 A/m² higher and 9.6 mm away from the location where it was found in the analogous non-pathological models. It was centered at (21.4,7.6,4) with a magnitude of 4.0 A/m². In the Pathological 10⁷εₒ solution, the maximum cortical current density was 0.9 A/m² higher and found 10.1 mm away from the location in the 5 kHz10⁷εₒ solution. It was centered at (20.1,8.6,3.8) with a value of 32.0 A/m².

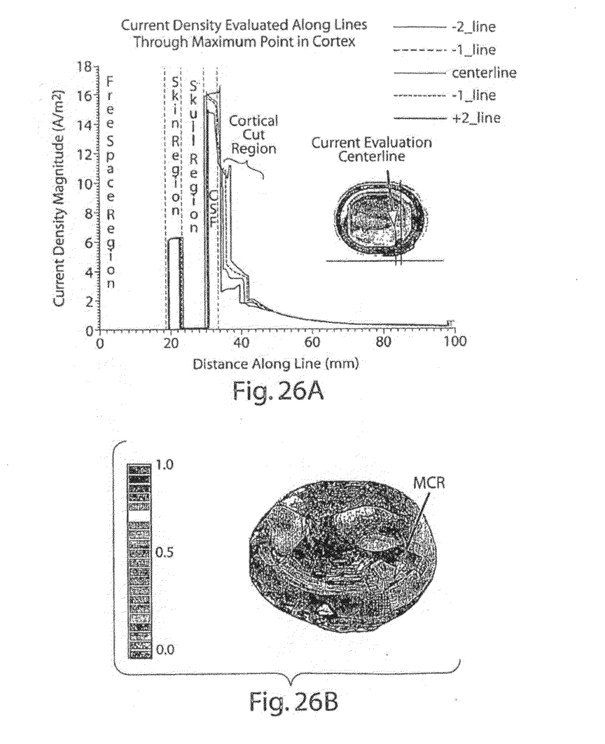

Stair step jumps in the current density occurred at each tissue boundary. However, there was inconsistency in the current density behavior at the location of the cortical pathology and the uniformity that was seen in the non-pathological cases (i.e., FIG. 23, plot A) was not present. In FIG. 26, plot A, the current density magnitudes are shown as evaluated along parallel lines that penetrate the model through the included tissues for the Pathological 10⁴εₒ solution. The line scheme is the same as that explained earlier and the centerline passes through the point of maximum cortical current density in the altered cortex.

The maximum values of the ratio of the normal to current density magnitudes in the Pathological solutions were higher in the region of the cortical cut compared to the analogous non-pathological solutions. For the Pathological 10²εₒ and Pathological 10⁴εₒ solutions, the maximum normal current ratio was 0.88, localized to an area of approximately 6.5 mm² centered at (16.4,−10.4, −4.1), 20.4 mm away from the cortical hot spot (see FIG. 26, plot B); the current density magnitude at this location was approximately 2.6 A/m², a value of 0.63 of the maximum cortical current density. In the region of maximum cortical current density, the maximum value of the ratio was approximately 0.36. For the Pathological 10⁷εₒ solution, the maximum normal current ratio was 0.73 localized to an area of approximately 14 mm² centered at (16.8,−9.3, −4.2), 19.9 mm away from the cortical hot spot; the current density magnitude at this location was approximately 18.4 A/m², a value of 0.58 of the maximum cortical current density. In the region of maximum cortical current density, the maximum value of the ratio was approximately 0.26. The results are tabulated in Table 3.

The foregoing describes the analysis of a realistic head model based on an MRI derived geometry integrated with variable tissue electric properties. The model provides evidence for the existence of currents normal to the cortical interface, demonstrates the effect of the tissue boundaries on the induced current, and allows one to test the predictions of alpha dispersion theory.

Whereas previous models, based on simplified geometries, predict the absence of currents normal to the cortical interface our model suggests that these currents do in fact exist and in some cortical regions are the dominant vector component. For the conditions modeled, normal current components accounted for approximately 30% of the current density in the region of the cortical hot spot. From this study, it appears that the ratio of the magnitude of normal current density to current density is maximal at locations that are sharply angled relative to the coil face. Thus, the geometry appears to be the determining factor in the generation of normal currents, which is logical, as geometrical simplifications were responsible for their absence in prior models.

This was most notable in the pathological solutions, where ratio values as high as 0.88 were centered on the surface of the cortical cut nearly perpendicular to the coil face. In the head models, the magnitude of the ratio decreased somewhat when displacement currents were present. Though somewhat difficult to generalize, the trends in the normal component magnitude depend both on the relative tissue to coil geometry and the electrical properties of the tissues. Therefore, theories that suggest that neurons that run parallel to the cortical interface are preferentially stimulated, based upon the premise that normal fields are negligible, need to be reassessed.

Applicant's model also provided data illustrating the tissue layer boundary's influence on the induced current densities. The conductivity of the tissue was the determining factor for the magnitude of the current density in all but the 5 kHz10⁷εₒ solutions (see FIG. 23, plots A and B for the 5 kHz10⁴εₒ and 100 Hz10⁷εₒ solutions). The capacitive effects of the tissues were negligible in these solutions, and the ratio of cortical current density to source strength was similar to that of the model of Roth. A value of (5.13×10⁻⁸ A/m² in the cortex)/(1 A/s source) was calculated in Applicant's model and a value of (1.13×10⁻⁷ A/m² in the cortex)/(1 A/s source) in Roth's model. The results were less clear-cut for the 5 kHz10⁷εₒ solutions (see FIG. 23, plot C), where the stair step jumps in the current density magnitude were primarily influenced by the permittivity of the tissue.

But in the case where the tissue permittivity values at the interface were equal, the conductivity influenced the change in the current density magnitude. This was the case at the gray matter/white matter interface, where the current density magnitude decreased in the white matter (the tissue with the lower conductivity) even though the gray and white matter shared the exact permittivity value. In the 5 kHz10⁷εₒ solution, we found a maximum cortical current density of 31.1 A/m² and a ratio of cortical current density to source strength of (5.51×10⁻⁷ A/m² in the cortex)/(1 A/s source). If the extreme values of alpha dispersion theory prove to be accurate, the current densities in the cortex will be higher than previously expected.

Additionally, it was found that the induced current densities were altered in the region of the cortical hot spot when the underlying geometry and tissue properties were modified. We can rule out this finding as a mathematical anomaly, because the Mirror Pathology solutions (where the model was modified at a region far removed from the cortical hot spot) showed no such field perturbation and were well characterized by the analogous 100 Hz10²εₒ-5 kHz10⁷εₒ solutions. Thus, our model has confirmed the results of earlier half plane studies, but now in a multi-tissue system that was more indicative of a true head geometry.

Fields were perturbed in the pathological solutions, relative to the non-pathological solutions, because the induced fields must satisfy a new set of boundary conditions in the region of the cortical hot spot. In our model we replaced the cortical cut with CSF, which provided a different conductive path for the induced currents away from the maximum current density location in the non-pathological model. The location of the maximum current density was shunted to the border of the cortical cut and found to increase in magnitude at the cut edge in the Pathological models. Furthermore, the current density magnitudes showed little consistency at the region of the cortical cut. Both of these results are relevant for TMS stimulation in populations with brain abnormalities, where the site of activation could be significantly modified. Experimental and modeling data suggests that the site of activation is predicted by the electric field in the cortical neurons.

For instance Nagarajan and Durand found for “short axons with sealed ends, excitation is governed by the boundary field driving function which is proportional to the electric field”. And Maccabee et. al., state “excitation at the terminations take place at much lower thresholds and it occurs at a site within the peak electric field” as does excitation at fiber bends of corticospinal and other neurons (note that this is different from the activation site of long (relative to the coil dimensions) straight neurons in the peripheral nervous system, which is predicted well by the first spatial derivative of the induced electric field). Applicant's data thus suggest that the site of activation can be altered when the geometry of the cortex is modified in the area of maximal cortical current density. For example, in the area of stroke recovery the stimulating field would be perturbed at the pathology site of patients with large infarctions and the location of stimulation could differ from that anticipated.

Finally, Applicant's model provided a means to test the predictions of alpha dispersion theories. For all but the 5 kHz10⁷εₒ solutions, the displacement currents were negligible. This could be predicted based on the tissue charge relaxation times discussed earlier. The time scale of the 5 kHz source (2×10⁻⁴ sec) was less than or on the same order of magnitude as the tissue charge relaxation times for the 10⁷εₒ tissue permittivity values, as illustrated in Table 4 below.

TABLE 4

| Relative | Conductivity | | | |
|---|---|---|---|---|
| Permittivity | (S/m) | | Relaxation time | |
| Magnitude-in terms | Low | High | constant(sec) | |
| of εₒ (F/m) | (skull) | (CSF) | Skull | Skin |
| 10² | 0.010 | 1.654 | 8.85e−8 | 5.37e−10 |
| 10⁴ | 0.010 | 1.654 | 8.85e−6 | 5.37e−8 |
| 10⁷ | 0.010 | 1.654 | 8.85e−3 | 5.37e−5 |

Thus in the 5 kHz10⁷εₒ solutions, the magnitude of the cortical current density was approximately one order of magnitude larger due to the contribution from the displacement current, but interestingly not far in location from the analogous solutions with negligible displacement current. Additionally, the results already discussed (i.e., normal currents to the cortical interface, tissue boundary constraints, and field perturbations) were still seen with the existence of displacement currents.

There is a consensus on an inverse trend of increased permittivity with decreased frequency and a clear distinction between the different tissues, but the overall permittivity magnitudes remains an area of active research. Most of the data would indicate that the tissue permittivity values are of the order 10⁴-10⁵εₒ above 1 kHz and that the extreme values in excess 10⁷εₒ are only found at frequencies below 100 Hz. From this, it is possible to conclude that displacement currents have no bearing on TMS. However this is stated with reservation, as magnitudes in excess of 10⁷ have recently been reported for in vivo measurements of the permittivity of muscle (which often show similar dispersive properties to tissue in the head and brain).

Moreover, most reported tissue permittivity values come from excised post mortem tissue, even though it has become evident that alpha dispersion effects becomes less prevalent after death and the permittivity values generally decrease. Thus, if these high permittivity values prove to be true, it would be necessary to reevaluate the quasistatic approximation used in prior field models and implement solutions which account for the displacement current in systems like the one proposed. Furthermore, this poses an entire new set of questions concerning the mechanisms of neural activation.

The FEM model developed herein can be used to address many of the early difficulties that existed in TMS modeling. Current density vector and magnitude plots were developed, highlighting the effects of the conductive boundaries. Tissue conductivity proved to be the main determinant of the current density magnitudes when the displacement current was negligible. With displacement currents, the permittivity was the main determinant of the current density magnitude. Additionally, the existence of displacement currents was shown to increase the maximum cortical current density by an order of magnitude if the extreme values of permittivity reported by some researchers prove to be accurate.

For all the solutions, currents normal to the cortical interface were evident; thus models that state that tangentially oriented neurons are preferentially stimulated, based upon the premise that fields normal to the interface are negligible, should be re-examined. Finally, the modification of the cortical geometry was shown to perturb the stimulating fields such that targeting of the cortex could be impaired in non-normal populations.

Additionally, the low frequency permittivity values may be more relevant than earlier models have suggested. If such high magnitudes of permittivity are truly seen in biological systems, than displacement currents have a role in TMS. In order to address these issues Applicant presents in-vivo measurements of the tissue conductivity and permittivity from surgical animals. And to coincide with the other issues raised above Applicant provides a further study related to how pathologies can alter these fundamental electrical tissue properties.

As discussed in the foregoing, there remains some debate as to the actual low frequency tissue permittivity and conductivity values that are exposed to TMS fields. It is believed that alpha dispersion occurs in biological tissues within the frequency range of TMS. Many researchers have experimentally addressed the issue and others have developed models of the process and reviewed dispersion in biological material. While the trend of increasing permittivity with decreased frequency is seen throughout the literature there is a limited consensus on the value of the relative permittivity magnitudes in biological tissues where values in excess of 10⁷ and as low as 10² have been reported in the TMS frequency spectrum. Thus, Applicant has recorded the low frequency conductivity and permittivity values in a living animal to ascertain the proper values.

To measure values, a set of medical grade stainless steel micro forceps were adapted to create the recording probe. The contact area (200-800 μm², as controlled by a micro-positioner) was coated with roughened platinum, to minimize the surface polarization effects. The contact separation was controlled by a contact screw, and locked to 50 μm during the tissue recordings. The remaining electrode surface was insulated.

Impedance measurements were made with an HP4192 A Impedance Analyzer. The contact area of the recording probe was fixed by the micropositioner and the contact screw. With the dimensions set, the permittivity and conductivity could be determined from the measured resistance and capacitance, this method of ascertaining the fundamental tissue values has been implemented by other researchers in both in-vitro and in-vivo studies. Recordings were taken from 10 Hz to 10 KHz, sweeping the log scale, for this experiment (note recordings were made into the MHz range but are not included in this document because they are outside of the frequency range of relevance of the thesis, but they will be published in a forthcoming document).

One adult female cat (3.4 kg, 5 yrs) was used for this study. The cat was initially anesthetized with 2.2 cc of Pentobarbitol. During the experiment, pentobarbital was applied relative to the metabolic function of the animal to maintain complete anesthesia. Heart rate (137-195 beats/min), respiratory rate (9-23 strokes/min), and rectal temperature (35.22-36.8 C) were all monitored and maintained within fairly close limits for the duration of the recording session which lasted approximately 18 hours post induction.

The head was then secured in a non-metallic stereotaxic frame (Kopf, Tujunga, Calif.). The animal's tissues were systematically exposed and recorded from. An average of 12 tissue recordings at separate locations were made per tissue (however with white matter, just 5 total recordings were made due to the difficulty in surgically exposing the tissue). The tissues recorded were the skin, gray matter, and white matter. Difficulties arose in recording from the CSF and it was not included in this study. During the last 6 hours of the experiment a hemorrhagic stroke was induced in the gray matter and systematically recorded from at 3 hours 30 minutes and 4 hours 30 minutes post infarct. Gray matter areas outside of the infarction zone were also recording from for comparison.

The tissues showed similar dispersion profiles as had been reported by other researchers, however the magnitudes were not the same as those reported in the literature (showing permittivities approximately 1 order of magnitude higher than those reported in the literature in the range of 1 to 10 kHz and different overall conductivities).

The conductivity and permittivity data are included in tabular form in the Appendix of Provisional Application Ser. No. 60/779,847, which was previously incorporated in its entirety, as Tables 3.1, 3.2, 3.3, and 3.4. In Table 3.5 in the Appendix of the '847 application, the Gray Matter values are provided for the tissue 3 hrs 30 min post stroke and in Table 3.6, in the Appendix of the '847 application, the Percentage Difference between the 3 hr 30 min post stroke recording and pre stroke recording is provided. In Table 3.7, in the Appendix of the '847 application, the Gray Matter values are provided for the tissue 4 hrs 30 min post stroke and in Table 3.8, in the Appendix of the '847 application the Percentage Difference between the 4 hr 30 min post stroke recording and pre stroke recording is provided.

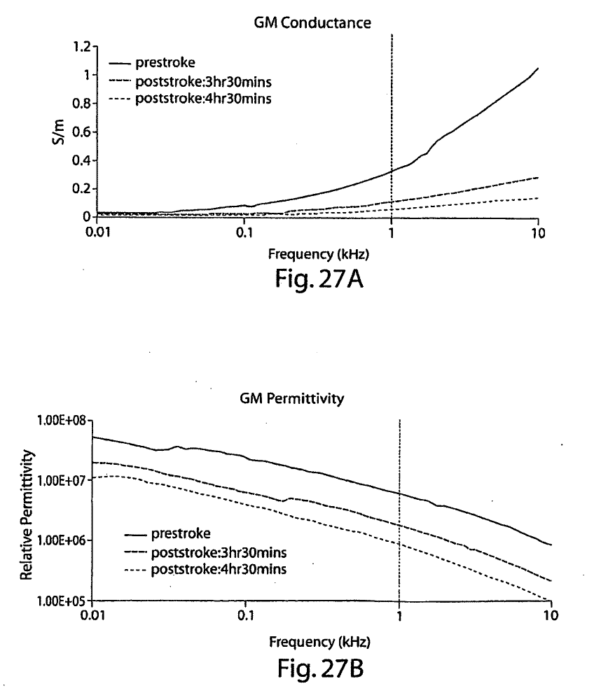

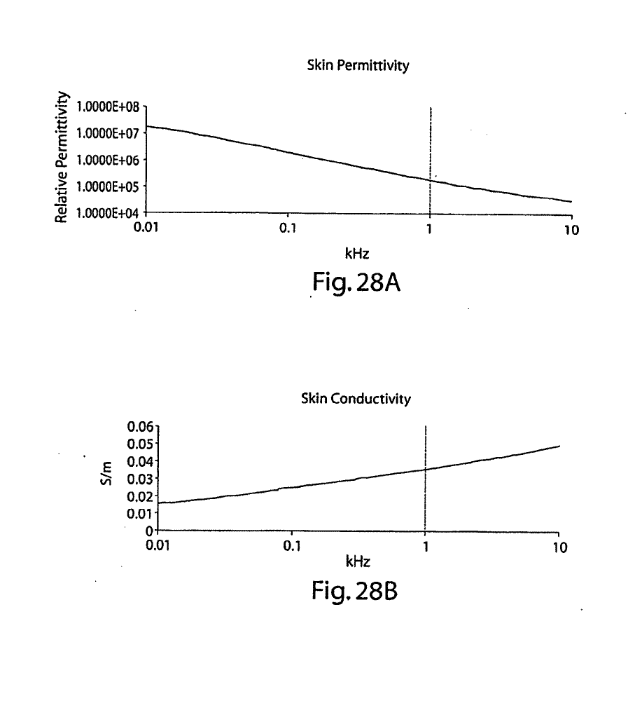

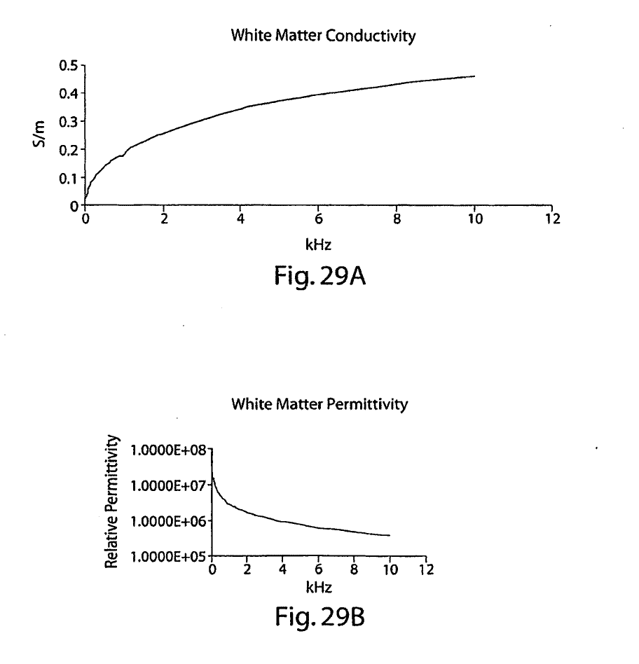

FIG. 27A displays the pre and post stroke gray matter conductance. FIG. 27B displays the pre and post stroke gray matter permittivity. The gray matter values outside of the infarction zone did not differ from the pre-stroke recording average by more than +/−5%. FIGS. 28A and 28B illustrate skin permittivity and conductivity, respectively. FIGS. 29A and 29B illustrate white matter conductivity and white matter permittivity, respectively.

As is evidenced by the results, the in-vivo values of the healthy tissue that were recorded in these experiments differed greatly from both published values and the values in the infarcted tissue. The infarcted tissue however was closer in magnitude to the published values in the literature. This could prove to be a significant fact, providing evidence that all prior values recorded from in-vitro tissue samples were inaccurate.

The permittivity values reported in the healthy tissue show less attenuation in the overall permittivity values as reported in excised tissue and the conductivities conversely increase more rapidly than reported in the literature. Overall, this results in values that are approximately 1 to 2 orders of magnitude higher in permittivity at 5 kHz. For instance, at 5 kHz we measured 1.76×10⁶ times the permittivity of free space, but the Brooks Air Force database reports values of approximately 3×10⁴ times the permittivity of free space.

In the infarcted tissue, the recorded values progressively diverged from those recorded in the healthy tissue. Both the permittivity and conductivity decreased with increasing time following the cerebral event. This is key, as most reported tissue permittivity values come from excised post mortem tissue, even though it has become evident that alpha dispersion effects becomes less prevalent after death and the permittivity values generally decrease. Our results once again confirm this prediction and for the first time show the results in an in-vivo situation.

Numerous researchers have proposed theories about the astronomically large permittivity levels. In 1957, Schwan modeled tissues as suspensions of charged particles and began explaining tissue effects in a way similar to researchers studying low frequency high amplitude permittivity values measured in electrolyte solutions and related the effects to counterion double layer effects. In 1987, Grosse and Foster presented a simplified model of these electrolyte effects and developed simplified equations of the permittivity that depend solely on diffusion of ions surrounding the charged particles in the solutions (dependent on the debye length, radius of the charged particles, and the angular frequency). Foster subsequently extended these concepts to tissue alpha dispersion effects. An alternate explanation was proposed by Dissado, whereby the cells of tissue (represented as capacitors and resistors in fractal networks) could be shown to generate extreme tissue values of permittivity.

As for TMS, large questions are raised by these results. If these reported values are indicative of in-vivo human tissue values, then the displacement current clearly has to be included during TMS modeling studies and a capacitive component needs to be included in the cellular models of stimulation. There is a very real possibility that displacement currents are a factor during TMS, and as such we will discuss its impact on analysis of the stroke and atrophy models in the later chapters modeling TMS stimulation. Ultimately, the inclusion of displacement currents will not change any of the overall trends of stimulation (location, perturbation effects, current density orientation) but rather the overall magnitude of the current densities. As such, the inclusion of the displacement currents may have the most direct impact on the neural cellular models of stimulation.

Since its inception, TMS has been widely used as a tool in stroke research for diagnostic, prognostic, and even therapeutic applications. TMS was first used in stroke patients in 1990. Since that time, several studies have been published using TMS as a diagnostic tool, prognostic tool and investigative tool for stroke pathophysiology. In addition, the modulatory characteristics of rTMS render it a realistic and promising technique to treat brain dysfunction after stroke.

However, to date, there has been no systematic study of the effects that strokes can have on perturbing the currents induced by TMS in the neural tissue, despite the well-known fact that after a stroke, numerous physiologic changes occur in the brain tissue, which can alter its electrical response properties. Necrotic brain tissue in the infarction region is phagocytized by inflammatory cells and replaced by a cerebral spinal fluid (CSF) filled network of astrocytes and glial fibers. The degree to which the infarction region fills with CSF is dependent on the degree of damage. This CSF influx represents a six-fold increase in conductance in the infarction region and a drastic modification to the tissue geometry and conductive matrix of the region. In TMS, similar changes in tissue geometry and electromagnetic properties have been shown to alter the induced stimulating currents in both phantom and modeling studies.

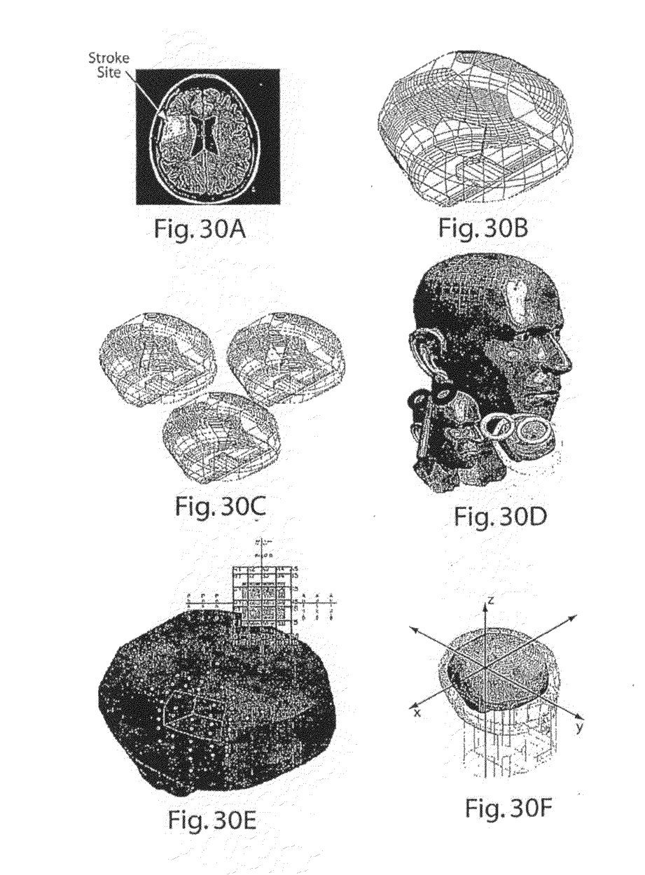

Multiple MRI derived finite element head models were constructed and evaluated for various stimulation orientations to address the effects that strokes can have on perturbing the induced stimulating TMS current. According to some embodiments, an initial sinusoidal steady state finite element model (FEM) was developed using the Ansoft 3D Field Simulator software package with the eddy current solver. We used an MRI guided three-dimensional CAD rendering of the human head to solve for the currents induced in the cortex during magnetic stimulation. We refer to this as the healthy head model, as illustrated in FIG. 30, and discussed in the foregoing. This model was generated to include the skin, skull, CSF, and cerebral tissue.

The tissue conductivities, while somewhat attenuated below 1 kHz stimulation frequencies, were considered essentially constant for the frequencies of TMS (the power spectrum of typical stimulators is composed of components less than 10 kHz). The greatest variability existed in the referenced values for the skin conductivity, most likely due to unaccounted anisotropies; however, all the tissues were considered isotropic and homogenous for this model. Thus, the tissue conductivities in the model were assigned the mean value from multiple references; skin at 0.465 S/m, bone at 0.010 S/m, CSF at 1.654 S/m, and cerebral tissue at 0.276 S/m (see Table 4.1 and Table 4.1-S, references included in table).

As discussed in chapter 3, the actual low frequency permittivity value of biological tissues is an area of ongoing investigation. Nevertheless, there is limited consensus on the exact value of the relative permittivity magnitudes in biological tissues. Although we found values with magnitudes in excess of 10⁷ εₒ, magnitudes in excess of the order 10⁴ εₒ are infrequently encountered in the range of peak TMS source frequencies in the literature. Thus, in this present study, the tissue permittivity was modeled as shown in Table 5 below. However, models were completed which included higher order permittivity values (10⁷ εₒ) and their impact is discussed in further detail below.

TABLE 5

| | | Relative |
|---|---|---|
| | Mean | Permittivity Scheme |
| Tissue | Conductivity (S/m) | (F/m) |
| Skin-Scalp | 0.465 | 1.2 × 10⁴ εₒ |
| Bone-skull | 0.010 | 0.8 × 10⁴ εₒ |
| Cerebral Spinal Fluid | 1.654 | 0.60 × 10⁴ εₒ |
| Cerebral Tissue | 0.276 | 1.2 × 10⁴ εₒ |

The source was modeled as a figure of eight coil with two 3.5 cm radius windings made of a single turn of 7 mm radius copper wire. The copper was modeled as a perfect conductor (currents constrained to the surface) with the permittivity set to εₒ, conductivity of 5.8×10⁷ S/m, and a permeability of 1.0μₒ. The source current was set at 5 kHz with a 1.8×10³ A peak current (5.65×10⁷ A/s, rate of change of the peak current with time). The 5 kHz frequency component of the source was chosen based on the peak frequency component seen in the power spectrum of the commercially available Magstim Rapid Stimulator. Unless otherwise noted, for each solution obtained, the coil was positioned with its facial plane tangential to the scalp, with its plane of symmetry (containing the coil handle) at an approximate 30 degree angle to the normal from the most lateral edge of the scalp surface, and with its facial plane 7 mm above the scalp surface to account for the insulating layer found in commercially available coils.

The FEM solver implemented a modified T-Ω method to solve for the induced current densities. The Ansoft FEM solver was set to follow an adaptive iterative process with convergence limits determined by the energy error in the system, further detailed in the foregoing. The criterion for model convergence was defined as an energy error below 1.0%, as illustrated in Table 2 above.

The magnitude and location of the maximum cortical current density were evaluated for each coil position tested for the healthy head model. Additionally, the surface area on the cortex where the current density was greater than 90% of its maximum value was calculated (i.e., if the maximum magnitude of the cortical current density was 1 A/m² for coil position X, then the area was calculated where the current ranged from 0.9-1.0 A/m²); we will refer to this as the maximum cortical current surface area. Furthermore, the induced current density vector behavior was also analyzed in the tissues. Where the results are reported as current density magnitudes, they indicate the magnitude of the sinusoidal steady state current density in the units of A/m², unless otherwise noted. Additionally the x, y, z coordinate system used in this paper is defined in FIG. 30, plot F and uses mm units.

Eleven different stroke models of various sizes and geometries were implemented to compare to the healthy head model under different conditions. These stroke models were constructed by altering the healthy head model's cortical geometry guided by the brain MRIs of patients with cortical strokes treated at Beth Israel Deaconess Medical Center. Strokes were chosen based on the different infarction volume and dimensions.

To represent the infarction site in the FEM construction, CSF was used to replace the damaged tissue as shown by imaging and histopathology studies in the post acute stage. The edges of the stroke were smoothed in the stroke model construction and the stroke volumes were considered homogeneous, excluding islands of cortical tissue inside the stroke regions. Stroke 1 had an approximate volume of 18.5 cm³ and was located in the right frontal lobe due to an infarction of superior branches of the right middle cerebral artery with a maximum 2.5 cm inferior to superior length along the cortical face, a maximum 3.5 cm anterior to posterior length along the cortical face, and a maximum depth of 2.8 cm measured from the cortical face (see FIG. 30, plots A and B).

Strokes 2A-2C were all located in the right frontal lobe due to infarctions of superior branches of the right middle cerebral artery with approximate volumes of 1.3 cm³, 2.6 cm³, and 5.3 cm³ respectively. Strokes 2A-2C all shared maximum depths of 4 mm measured from the cortical face, 4 cm inferior to superior lengths along the cortical face, and a 1 cm, 2 cm, and 4 cm anterior to posterior length along the cortical face respectively (see FIG. 30, plot C).

Strokes 3A-3C were all located in the right frontal lobe due to infarctions of superior branches of the right middle cerebral artery with approximate volumes of 2.6 cm³, 5.3 cm³, and 10.6 cm³ respectively. Strokes 3A-3C all shared maximum depths of 8 nun measured from the cortical face, 4 cm inferior to superior lengths along the cortical face, and a 1 cm, 2 cm, and 4 cm anterior to posterior length along the cortical face respectively. Strokes 2A-2C differed primarily by depth from the corresponding stroke 3A-3C models (i.e., Stroke 2A and Stroke 3A shared the same shape along the cortical face but differed by the maximum depth, see FIG. 30, plot C).

Strokes 4A-4C were all located in the right frontal lobe due to infarctions of superior branches of the right middle cerebral artery with approximate volumes of 5.3 cm³, 10.6 cm³, and 21.2 cm³ respectively. Strokes 4A-4C all shared maximum depths of 12 mm measured from the cortical face, 4 cm inferior to superior lengths along the cortical face, and a 1 cm, 2 cm, and 4 cm anterior to posterior length along the cortical face respectively. Strokes 4A-4C differed primarily by depth from the corresponding stroke 2A-2C and 3A-3C models. Additionally, the infarction sites for stroke models 2-4 (Strokes 2A-2C, Stroke 3A-3C, Strokes 4A-4C) were normalized in location to share the same center on the model's cortical face.

Finally, Stroke 5 represented a large stroke due to left MCA occlusion; its size was approximately 350 cm³ and was designed by removing the left cortical hemisphere from the model. The stroke models differed from the healthy head model only at the infarction location; where the electrical properties corresponded to that of CSF at the infarction site as opposed to gray matter, and were identical to the healthy head model outside the stroke region (both geometrically and electrically). The stroke dimensions are tabulated in Table 6 below.

TABLE 6

| | Maximum | Maximum | |
|---|---|---|---|
| | Anterior to | Inferior to | Maximum |
| Stroke | Posterior Width | Superior Length | Depth |
| Model | (mm) | (mm) | (mm) |
| Stroke 1 | 35 | 25 | 28 |
| Stroke 2A | 10 | 40 | 4 |
| Stroke 2B | 20 | 40 | 4 |
| Stroke 2C | 40 | 40 | 4 |
| Stroke 3A | 10 | 40 | 8 |
| Stroke 3B | 20 | 40 | 8 |
| Stroke 3C | 40 | 40 | 8 |
| Stroke 4A | 10 | 40 | 12 |
| Stroke 4B | 20 | 40 | 12 |
| Stroke 4C | 40 | 40 | 12 |

| | |
|---|---|
| Stroke 5 | Left hemisphere removed |

The magnitude of the maximum cortical current density, the location of the maximum cortical current density, the maximum cortical current surface area, and the induced current density vector behavior for each of the coil positions in the stroke models were analyzed.

The stroke solutions were then compared to the analogous healthy head models. The number of cortical areas in the stroke models where current density magnitudes were found to be in excess of 100%, 120%, and 150% of the maximum cortical current density in the corresponding healthy head model were calculated. For each of these areas the location, the maximum current density magnitude in the area, and cortical surface area were also calculated.

Specific tests were conducted to explore the effects of the relative coil to stroke location, the infarction size and shape, and large contralateral strokes can have on the induced stimulating currents. In order to investigate the effects of the coil location on the induced stimulating current, solutions were obtained for both the healthy head model and stroke 1 model with the coil center placed at locations along a grid on the scalp surface (see FIG. 30, plot D). As the exact location of the maximum current density on the cortex could not be predicted prior to solving the problem, the coil locations were chosen based on the site of the normal projection from the figure-of-eight coil's center on the scalp surface to the cortical surface (much like the prediction method used in many commercially available frameless stereotactic systems), with enough locations to broadly encompass the stroke border and allow for the testing of locations both inside (12 locations) and outside (38 locations) the infarction region.

Points A1-A5 are located along a horizontal line that transverses the scalp surface such that their projections are approximately 1 cm superior to the superior stroke border. Similarly, the projection of points B1-B5 and C1-C5 are approximately 0.5 cm superior and 0.5 cm inferior to the superior stroke border respectively. The projection of D1-D5 and E1-E5 are 0.5 cm superior and 0.5 cm inferior to the horizontal midline of the stroke outline. Finally, the projection of points F1-F5, G1-G5 and H1-H5 are 0.5 cm superior, 0.5 cm inferior and 1 cm inferior to the inferior stroke border. A similar arrangement exists for the projections of each of the vertical lines in the grid (A1-H1 through A5-H5) with reference to the anterior stroke border, vertical midline and posterior stroke border.

Additional locations were evaluated outside the grid border; three locations approximately 1.0 cm, 2.0 cm, and 3.0 cm posterior to the horizontal center of the posterior stroke border, and anterior to the horizontal center of the anterior stroke border, and two locations approximately 2.0 cm and 3.0 cm superior to the vertical center of the superior stroke border, and inferior to the vertical center of the inferior stroke border. We shall refer to these locations using the abbreviated position (Ant, Pos, Sup, Inf) and the distance so that Ant1.0 refers to the anterior point 1 cm from the anterior stroke border. One hundred total locations were evaluated, fifty for each of the stroke 1 and the healthy head models (see FIG. 30, plots D-E for the scalp surface grid and the cortical projections, Table 4.2 Supplementary in the Appendix of the '847 application tabulates all of the coil center positions on the scalp, and FIG. 30, plot F defines the coordinate system used for all of the models).

In order to investigate the effects of the infarction size and shape on the induced stimulating current, solutions to the FEM model were obtained for strokes 2A, 2B, 2C, 3A, 3B, 3C, 4A, 4B, and 4C and compared to the healthy head model with the normal projection from coil's center located over the center of infarction's cortical face and located approximately 0.5 cm posterior to the posterior border of each infarction. The coil position referred to as CPCenter corresponded to the center of the strokes' cortical faces (figure of eight coil centered at (−9,−3,−14) on the skin surface). CPA was 0.5 cm posterior from the stroke 2A, 3A, and 4A's infarction border (figure of eight coil centered at (−9,−15,−12) on the skin surface). CPB was 0.5 cm posterior from stroke 2B, 3B, and 4B's infarction border (figure of eight coil centered at (−6,−20,−13) on the skin surface). And CPC was 0.5 cm posterior from the stroke 2C, 3C, and 4C's infarction border (figure of eight coil centered at (−5,−25,−14) on the skin surface).

In order to investigate the effects a stroke can have on contralateral cortical stimulation, solutions were obtained for the stroke model 5 and the healthy head model where the coil was placed at locations of increasing distance from the removed hemisphere on the contralateral scalp surface, at points Sup3.0, Sup2.0, A3, B3, C3, D3, E3, F3, G3, and H3.

Every model converged below the 1.0% energy error stopping criteria. The average number of tetrahedron for the 50 healthy head model solutions was 168,471; and 179,311 for the stroke 01 solutions, 148,001 for the stroke 2A-stroke 4C solutions, and 142,403 for the stroke 05 solutions. Multiple machines, of varied computing resources, were used in the solution process; however, typical convergence times for a dual 3 GHz Xeon processor machine with 4 Gigs of Ram were as follows: 2:51:44 for the healthy head model with the coil in the C3 position, 2:57:38 for the stroke 01 model with the coil in the C3 position, and 2:42:39 for the stroke 05 model with the coil in the C3 position.

For the healthy head model, the maximum cortical current densities ranged from 1.45-4.35 A/m². The maximum cortical current surface areas, defined as the cortical surface area where the current ranged from 90% to 100% of its maximum, ranged in area from a focal 37 mm² to wide spotty areas equal to or in excess of 200 mm². For all of the solutions, the location of the maximum cortical current density did not correspond directly to the location of the normal projection from figure-of-eight coil's center, but the projection intersected the cortex within the maximum cortical current surface area. The induced current density variation and vector behavior seen in the tissues were consistent with those of previous studies where the vector orientation followed a figure-eight path with the greatest irregularity at the tissue boundaries [36, 40, 108]. In Table 4.2 Supplementary in the Appendix of the '847 application, the magnitude, location, and area of the maximum cortical current densities are tabulated for the 50 coil positions tested in the healthy head model.

When compared to the healthy head model, the current density distributions in the region of the maximum current density were similar in the healthy head model and stroke 01 model for only the Sup3.0, Sup2.0, Ant3.0, and Ant2.0 coil positions. For these solutions, the locations of the maximum cortical current density were found within 1.5 mm to 2.6 mm of the locations of the maximum cortical current density in the healthy head model and differed in magnitude from −6.1% to 5.1% of the maximum magnitude in the healthy head model.

However, the current density distributions of the stroke 01 model were considerably altered for the remaining 46 out of the 50 coil positions tested as compared with the healthy head model; whereby, the maximum magnitude, maximum location, and current vector orientations of the induced current density were altered in the stroke model. For these solutions, the maximum cortical current density was systematically found along the stroke borders within +/−1 millimeter of one of eight discrete locations; the superior anterior stroke border (SASB), superior midline stroke border (SMSB), superior posterior stroke border (SPSB), inferior anterior stroke border (IASB), inferior anterior to midline stroke border (IAMSB), inferior midline stroke border (IMSB), inferior posterior to midline stroke border (IPMSB), and the inferior posterior stroke border (IASB).

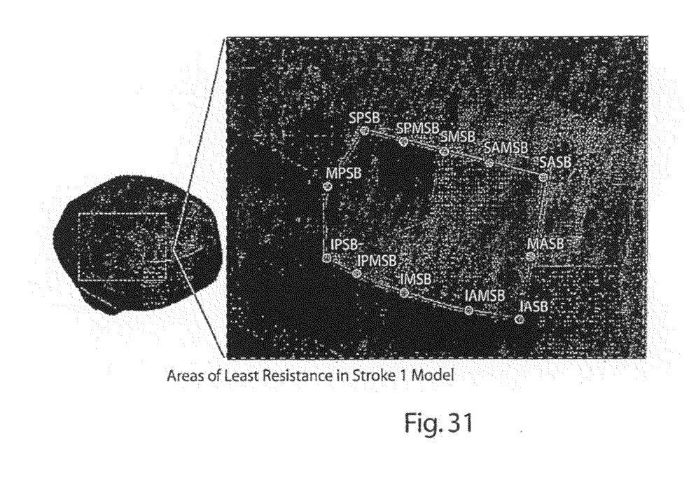

FIG. 31 illustrates all of these locations. In particular, FIG. 31 illustrates that the areas of least resistance in the stroke 01 model were found along the edge of the infarction region. The maximum cortical current density and center points of the areas of perturbation were found within +/−1 millimeters of one of these eight locations for 46 of the 50 coil positions tested. They included SASB at (−24.7,9.0,−16.6), SMSB at (−18.7,−4.2,−16.0), SPSB at (−15.1,−20.5,−17.0), IASB at (−11,16.4,−35.5), IAMSB at (−9,11,−35.3), IMSB at (−4,−1.5,−35.3), IPMSB at (−3,−6,−35.4), IASB at (−11,16.4,−35.5), MASB at (−15.6,16.5,−22.7), the SPMSB at (−18.2,−9,−16.2), and the SAMSB at (−22.2,31,−16.2).

These maximum cortical current density locations ranged from 3.8 to 28.5 mm distant from the corresponding location in the healthy head model and were generally located at the corner location along the stroke border that was most proximal to the figure-of-eight coil's center. Exceptions were found in cases where the maximum was located along the edge of the most proximal stroke border location instead of at a stroke corner location. Additionally, the maximums for the three most posterior coil positions evaluated along the E line (E1, E2, and E3) were located at the SPSB even though the inferior border was more proximal to the coil center (see Table 4.2-S). For these 46 coil positions, the cortical current density magnitudes were consistently larger than those found in the healthy head model, ranging from 1.85-7.42 A/m² while the differences between the healthy head and stroke 1 models ranged from 12.8% to 187.8%.

The maximum current density in the stroke cases for these 46 coil positions was localized to discrete focal sites along the infarction border. The maximum cortical current surface areas for these solutions were more focal than those of the healthy head model, ranging in area from less than 10 mm² to 77 mm² (i.e., the maximum cortical surface area was defined as the surface area on the cortex where the current density was greater than 90% of its maximum). Despite this result, the areas in the stroke models, where the current density magnitude was in excess of the corresponding healthy head maximum, were much larger in area than these maximum cortical current density areas.

Moreover, the areas in the stroke models where the current density magnitude was in excess of the corresponding healthy head maximum extended well beyond the discrete locations identified as the maximum point locations. For each of these 46 coil positions, there were at least two areas in the stroke 01 model where the cortical current density was greater than the maximum cortical current density of the corresponding healthy head solution, ranging in surface area from less than 10 mm² to as high as 284 mm².

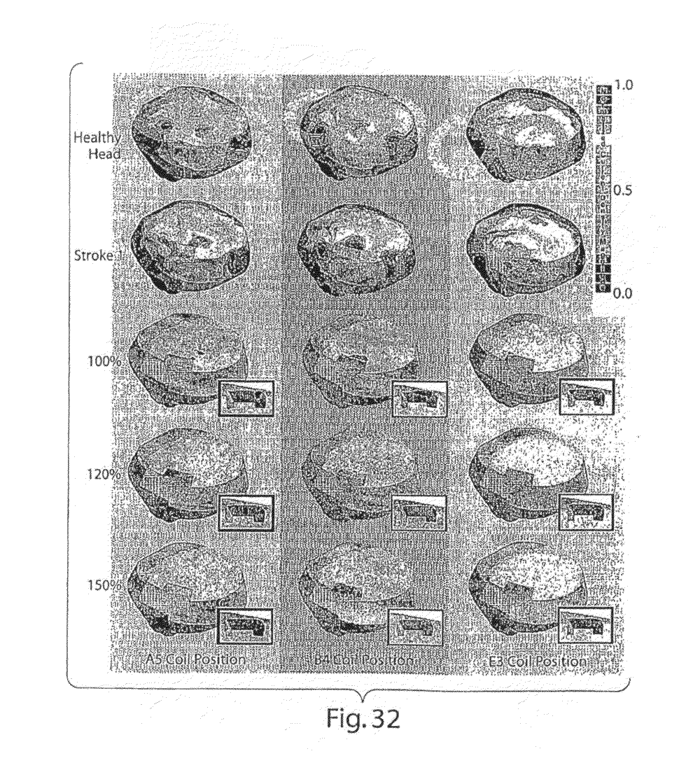

FIG. 32 illustrates current density magnitudes and Perturbations for the A5, B4, and E3 coil positions. The top plot of each column is that of the current density magnitude for the corresponding healthy head model, note that the scale is normalized to the corresponding healthy head model maximum current density magnitude. The second plot is that of the current density magnitude for the stroke 01 model, note that the scale is normalized to the maximum of the current density magnitude of the healthy head model such that everything above the maximum in the corresponding healthy head model is shown in bright red to highlight the perturbation effect. The third plot is that of the current density magnitude in the stroke 01 model which is greater than the maximum current density magnitude of the healthy head model. The fourth plot is that of the current density magnitude in the stroke 01 model which is greater than 120% of the maximum current density magnitude of the healthy head model. The fifth plot is that of the current density magnitude in the stroke 01 model which is greater than 150% of the maximum current density magnitude of the healthy head model.

For example in FIG. 32 for the B4 coil position, there were 3 locations where the current density magnitude in the stroke 1 model was greater than the maximum current density in the healthy head model. The first one was located along the upper stroke border from the SASB to the SMSB (both inside and outside the cortical cut) with an area of 57 mm² and a maximum current density magnitude of 3.48 A/m. The second one was located at the SPSB with an area of 9 mm² and maximum current density magnitude of 2.77 A/m², and the third was located at the MASB with a 5 mm² area and a 2.36 A/m² magnitude.

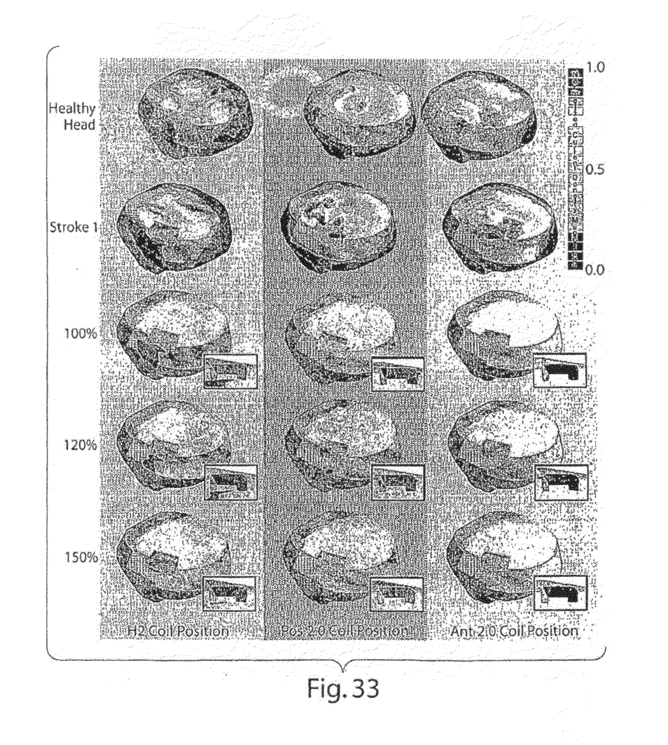

FIG. 33 illustrates current density magnitudes and perturbations for the H2, Pos 2.0, and Ant 2.0 coil positions. The top plot of each column is that of the current density magnitude for the corresponding healthy head model, note that the scale is normalized to the corresponding healthy head model maximum current density magnitude. The second plot is that of the current density magnitude for the stroke 01 model, note that the scale is normalized to the maximum of the current density magnitude of the healthy head model such that everything above the maximum in the corresponding healthy head model is shown in bright red to highlight the perturbation effect. The third plot is that of the current density magnitude in the stroke 01 model which is greater than the maximum current density magnitude of the healthy head model. The fourth plot is that of the current density magnitude in the stroke 01 model which is greater than 120% of the maximum current density magnitude of the healthy head model. The fifth plot is that of the current density magnitude in the stroke 01 model which is greater than 150% of the maximum current density magnitude of the healthy head model.

The five additional graphical examples provided in FIGS. 32 and 33, including the analysis for areas in the stroke 01 model where the current density was greater than 120% and 150% of maximum in the corresponding healthy head model. Table 4.3 Supplementary in the Appendix of the '847 application tabulates the averaged area information.

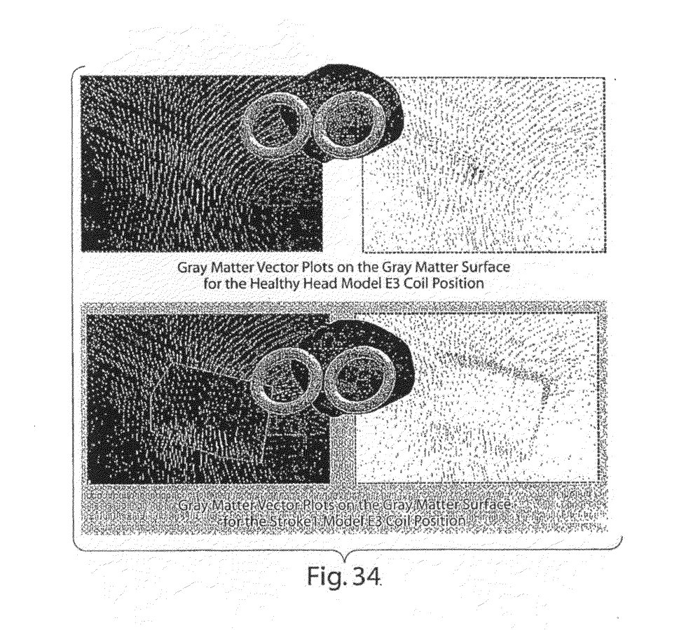

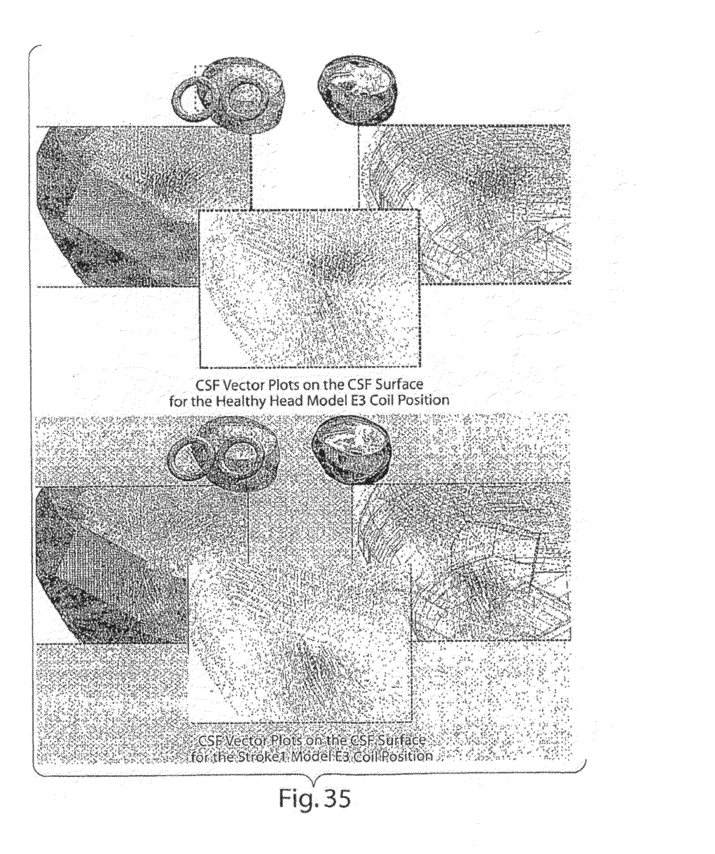

The induced current density distributions showed the greatest variation along the tissue boundaries, as had been reported earlier in a similar model and in a saline phantom model. In the stroke model, the current density vector distributions deviated from predictable figure-of-eight distributions that were seen in the healthy head model to conform to the infarction boundaries such that the current vectors became more perpendicular to the stroke boundary along its border and particularly focused at the corners where the areas of maximum cortical current density were found. For example, in FIGS. 34 and 35 the vector behavior is graphically displayed for the healthy head and stroke 01 model for the E3 coil position.

The altered directions of current vectors along the stroke borders are evident where vectors deviate in direction perpendicular to the stroke borders. Greater deviations are found along the anterior border, and into the corner points along the stroke border, most noticeably as the SPSB and SASB. Additionally, the current vector behavior in the CSF showed a similar type of perturbation. Although more subtle in the CSF, the current was still directed from its predictable course in the healthy head models towards the underlying stroke borders. In FIG. 35, the current density vector distribution in the CSF is plotted for the healthy head model and the stroke 01 model with the coil in the E3 position.

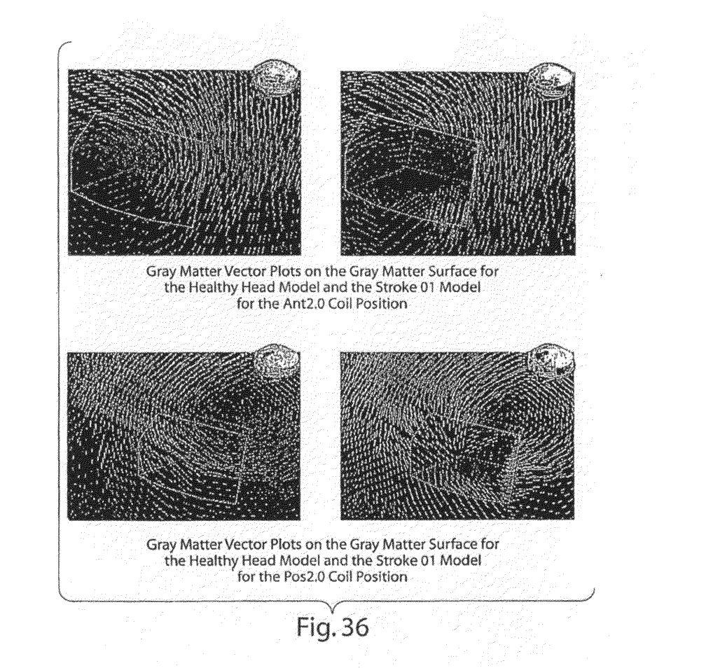

In the solutions with little to no perturbation (the Sup3.0, Sup2.0, Ant3.0, and Ant2.0 coil positions), the current density was attenuated and the current vector distribution was oriented tangent to the infarction border at the impending infarction region in the corresponding healthy head model. This could most easily be seen when one compares the Ant2.0 to the Pos2.0 solutions, see FIG. 36. The relative coil to head geometry was such that Ant 2.0 current distribution was localized to a region removed from the stroke border and the current density distribution in the healthy head model was sufficiently attenuated at the location of the infarction site in the stroke 01 model. The current density magnitude decayed to 44.0% of its maximum at a point 0.5 cm posterior to the anterior stroke border and 0.5 cm inferior to the location of the superior stroke border in the corresponding healthy head solution.

However, for the Pos 2.0 solution, the current density distribution was not sufficiently attenuated at the location of the infarction site in the stroke 01 model, decaying to 73.6% of its maximum at a point 0.5 cm anterior to the posterior stroke border and 0.5 cm inferior to the superior stroke border in the corresponding healthy head solution. Additionally, when comparing the vector orientations of these two coil positions for both the healthy head model and the stroke 01 model, the vector current orientation of the healthy head model was approximately normal to the location where the nearest infarction border was found in the stroke 01 model for the Pos 2.0 solutions and the vector current orientation of the healthy head model was approximately parallel to the location where the nearest infarction border was found in the stroke 01 model for the Ant2.0 solutions.

The degree of perturbation, both in area and magnitude, for the stroke 01 model was clearly dependent on the coil location. The relative head geometry/curvature, relative coil to infarction position, and which infarction border/discrete maximum current density location was closest to the coil center (or the parallel edge of the coil border when the center was distant) were the most obvious factors influencing the degree of perturbation. The eight discrete locations along the infarction border where the current maximums were found appear to correspond to areas of least resistance in the stroke 01 model. On average, the largest areas of perturbation and % differences between the healthy head model and the stroke 01 model were found along the inferior stroke border, these were found for the coil positions located along the flatter temporal face of the head where less focal distributions were seen in the corresponding healthy head model. As the source position and model (stroke 01 vs healthy head model) changed, the distributions obviously changed, but the largest degrees of perturbation were generally seen when the distribution in the healthy head model was such that the current density vector distribution was oriented normal to the location where the stroke border was found in the corresponding stroke 1 model.

Herein, we focus on the differences between the stroke 2A-stroke 4C models based on their geometries. Although similar trends were seen in the analysis of the stroke 2A-stroke 4 C models as were seen in the stroke 1 models regarding the degree of perturbation, the current vector behavior, areas of least resistance, and boundary effects, we limit their reporting here to highlight the major differences seen between the stroke 2A-stroke 4 C models.

The difference between the maximum current density magnitudes in the healthy head model and the corresponding stroke model got larger as the stroke depth got larger. Conversely, as the stroke shapes increased in anterior to posterior width, the magnitude differences between the healthy head model and the corresponding stroke got smaller. In general, as the stroke border got larger in width and/or depth, the areas of perturbation got larger. The results are tabulated in Table 4.4 Supplementary in the Appendix of the '847 application.

The % difference between the maximum current density in the healthy head and the stroke model decreased with increasing coil distance from the infarcted hemisphere. For all of the stroke 5 coil positions, except for that of the Sup3.0 position, there was only one area of perturbation for each position where the current density magnitude exceeded the maximum current density in the corresponding healthy head model (corresponding to the location of the new maximum); this area of perturbation decreased with increasing distance from the infarcted hemisphere. Additionally, for every coil position, except for the Sup3.0 coil position, the location of the coil density maximum was within +/−2 mm of the maximum location in the corresponding healthy head model. For the Sup3.0 position there were 2 areas of perturbation which exceeded 100% of the maximum in the corresponding healthy head model; at the location of the maximum cortical current density and where the maximum cortical current density was located in the corresponding healthy head model.

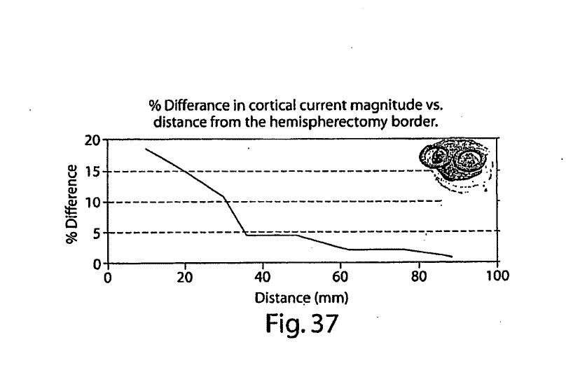

Similar vector behavior was seen in both the healthy head and stroke model for all of the solutions along the cortical surface except at the hemisphere border. These variations became more pronounced as the % difference between the models increased (i.e., the distances between the coil and the hemispherectomy diminished). The current vectors were oriented in a more perpendicular direction along the hemisphere border than they were oriented in the corresponding locations in the healthy head model. Table 4.5 Supplementary in the Appendix of the provisional application tabulates the results from these models. FIG. 37 plots the % difference between the healthy head model and the stroke 5 model vs. increasing coil distance from the stroke boundary.

The models discussed in the foregoing are based on a finite element electromagnetic solver integrated with MRI derived head models. The foregoing illustrates that the disruption due to a stroke can drastically modify the effect of TMS in several ways: (1) it alters the location of the maximum cortical current density, (2) it alters the magnitude and distribution of the induced currents, and (3) it modifies the focus of stimulation, all of which will alter the population of neural elements stimulated and ultimately lead to clinical implications, which are discussed below.

Various studies performed by Applicant and discussed in the foregoing demonstrate that in the presence of a chronic, cortical stroke, the predicted location of the maximum current density induced by TMS in the cortex based on conventional models would be inaccurate when stimulation is aimed to a cortical region proximal to the lesion. For the healthy head models, the perpendicular projection from the coil's face center was always within the area of the maximum cortical current. For the stroke models this was rarely the case; for all of the stroke models, where the source coil (figure of eight coil with two 3.5 cm radius windings) was centered within 1 cm of the infarction border, we found sizeable distances between the location of the maximum current density in the stroke and the corresponding healthy head solutions (distances between the corresponding maximums as great as 24.1 mm were observed).

However, this is not to say that the maximum location would be unperturbed when the coil center is greater than 1 cm away from the infarction border. For example in the stroke 1 model, there were coil positions where the coil center was 2 and 3 cm away from the stroke border in which the maximum location was still significantly altered. Thus, the exact distance will always depend on the coil size, relative coil to scalp angle, and head and infarction geometry.

Nevertheless, Applicant's present findings suggest that the current density distribution will not be appreciably altered in cases where the induced distribution is noticeably attenuated at the corresponding location in the healthy head model (in our studies the induced current density magnitude was found to be less than 50% of the maximum in the corresponding healthy head model at the location of the impending infarction site for cases that did not show a noticeable perturbation). This result was confirmed in all the solutions where the projection of the coil (both its center and border regions) onto the cortex did not overlap with the underlying infarction border (see the stroke 5 results).

The analysis also indicates that the magnitude of the induced current density is altered following a stroke and that the magnitude calculated in a healthy cortex is an inadequate predictor of the levels expected in pathological cases. Our modeling results suggest that the influx of CSF into the infarction region following a stroke essentially creates a new shunting route for the currents to follow. The CSF is far more conductive than the surrounding brain tissue and, thus, areas of least resistance appear to attract the current along the border of the infarction. Depending on the stroke geometry and coil location, multiple areas of least resistance may appear. These tend to be at corner points of the stroke boundary, a result often seen in the analysis of eddy currents at 90 degree corner points.

The current density difference between the healthy head model and the stroke models is large in especially focal regions (as high as 188% in the stroke 01 model for the G5 coil position). However, for some of these very focal areas of maximum cortical current density, the extreme perturbations would most likely have clinically insignificant effects. The rationale underlying this lack of effect is that these areas were confined to either corners or edges along the stroke border and generally spanned areas of less than 10 mm² where scar tissue and nonfunctional neural tissue would be found.

Nevertheless, as illustrated by multiple stroke models, these areas can also extend past the infarction border, at locations of presumed viable healthy cortex, such that these perturbations would have clinically significant effects. Additionally, for those models where the areas of maximum cortical current density were small, there were still large regions where the current density distributions were greater than those seen in the corresponding healthy head model—just not as great as the extremes found in the corner points (for instance in FIG. 33 for the Pos 2.0 coil position there are three locations where the current density magnitude was greater than the maximum found in the healthy head model ranging in area from 23 to 64 mm², yet the area of the maximum cortical current density was just 5 mm²).

Finally, there were numerous stroke solutions where the cortical current density was greater than that seen in the corresponding healthy head model in corresponding regions, even though it did not exceed the absolute maximum. These changes in the current density magnitude make the use of the MEP threshold unreliable as a TMS safety standard in the affected cortex and ultimately make the case for the pursuit of improved TMS safety standards in the altered cortical tissue. MEP thresholds developed in the region of altered cortical cortex should not be transposed as a reference to other cortical sites (and vice versa).

In addition to the potential location and magnitude inaccuracies, the analysis provides evidence that the focus of induced current from TMS will be appreciably diminished following a stroke. In the healthy head model the maximum cortical current density was always confined to a single discrete cortical location. However in the stroke models, there were many solutions with multiple disjoint areas, around the infarction border, where the current density was near its maximum or greater than the maximum seen in the corresponding healthy head models. For example in FIG. 33 for the B4 coil position, there are two areas separated by approximately 1 cm located at the SASB and the SMSB of 3.48 and 3.23 A/m² magnitudes.

Experimental and modeling data suggests that the site of activation is predicted by the peak electric field, and thus the cortical current density, in the cortical neurons. Applicant's data suggests that the site of activation will be altered in stroke patients (and in all patients who suffer from pathologies that alter the electrical and anatomical tissue properties proximal to the simulation site). In addition to the stimulation changes expected due to the new maximum cortical current density magnitude and location, different neural elements would also be activated in stroke patients due to the alteration of the current vector orientations. A modification of the current density vector orientation along the infarction border is predicted from the boundary condition that the normal current density components must be continuous across boundaries, when displacement current is negligible.

Thus, when one goes from the highly conductive CSF to the less conductive cerebral tissue the jump in normal components is expected, when compared to healthy tissue where there has not been a CSF influx. This will alter the current density direction along the stroke border and although there are different theories concerning the direction of the current vectors and which cortical neurons are stimulated, it is clear that the directionality of the induced currents plays a clear role in which neurons are stimulated. Thus, different neural elements could be activated in stroke patients than would be predicted in a healthy head.

The altered location of the maximum cortical current density location is important when TMS is used in the investigation and treatment of patients with stroke. When TMS is applied over the undamaged hemisphere or the damaged hemisphere (but where the coil is not proximal to the lesion location), the maximum cortical current density location can still be roughly predicted based on the expected results in a healthy head model. However when targeting regions of the cortex proximal to the lesion site, one would need to account for the perturbation of the maximum current density location. One solution to this problem would be the use of a stereotaxic system integrated with a field solver to predict the location of the maximum cortical current density during stimulation. Otherwise, the inaccuracies in the predicted location of the maximum cortical current density could prove to be dangerous or at the very least provide unpredictable results in behavioral or clinical applications of TMS.

TMS safety standards based on expected current density distributions for healthy cortices should not be applied in the altered cortical tissue. For example, it has been noted that stroke and other pathologies could lower the TMS seizure threshold in patients; and, in cases where the pathological tissue leads to amplification of the induced current density magnitudes the dangers are obviously magnified and the likelihood of seizure is increased. Additionally as discussed above, the changes in the current density magnitude make the use of the MEP threshold unreliable as a TMS safety standard in the effected cortex, as has been recommended in studies where current perturbations are not expected.

Overall, the demonstrated cortical current perturbations should be considered when interpreting clinical TMS studies of stroke. For example, in the area of brain plasticity, TMS has been one of the tools used to demonstrate changes in motor map size, location, and excitability after brain lesions such as stroke. For instance, Delvaux et al (2003), studying 31 patients who experienced an ischemic stroke in the middle cerebral artery territory, showed that persistence of MEP on the affected side just after the onset of the stroke was a strong predictor of good recovery. Furthermore, this author observed a significant displacement of center of gravity of motor maps towards more frontal regions on the affected side while no change was noted on the unaffected side. Our results suggest that TMS based studies of cortical changes following a stroke can be influenced by the disturbance of the induced electric current caused by physiological changes to the lesion area. Therefore, these patients may not have presented MEPs not because of the lesion characteristics, but because the induced stimulating current disturbance caused by the lesion. This is not to say that MEP measurements cannot provide a gauge of the remaining viable corticospinal projections, but ultimately a clinical prognosis based on the MEP response should take into account the effect of the lesion on the induced electric current by TMS.

Another area where the current perturbations need to be considered is in the field of TMS induced neuro-rehabilitation. TMS stimulation has been proposed as a tool to affect plasticity in stroke patients. The neuro-rehabilitative capacities of TMS have been demonstrated in the undamaged hemisphere in stroke patients. However, there have not been any studies that offer clear results with stimulation focused in the lesion region. This is quite possibly due to limitations of the current density perturbations. In future studies focused on stimulation of the infarcted cortex, the perturbations will need to be accounted for preferably in a stroke by stroke basis, due to the unique geometry of each infarction. The fact that the degree and location of perturbation will be uniquely determined by the relative lesion shape to coil position means that the perturbations should be accounted for with a stereotactic system integrated with a MRI based field solver which accounts for the tissue changes. Systems which predict the location of stimulation based on the perpendicular projection from the coil face will not be accurate. Additionally, systems that transpose perfect sphere models or homogenous geometries onto MRI based trackers will suffer from the same unreliability.

If the stereotactic field solver technology is not available, there are no simple rules of thumb which will predict the exact perturbations expected. However from this model, one could expect the currents to be amplified on the stroke border proximal to the coil boundary, especially at locations where there are sharp demarcations in the tissue type. For instance, in our model sharp edges on the stroke border seemed to serve as areas of least resistance. Oftentimes we found that the areas of elevated current magnitude extended from the expected stimulation location to these areas of least resistance on the lesion border with the largest perturbations proximal to the figure-of-eight coil's center. However, cases were found where the outer edge of the coil was proximal to the largest perturbations, especially when the coil was positioned such that the currents were oriented normal to the stroke interface. As such, one would prefer to place the coil such that no part of it was overlying the stroke border in order to minimize the perturbations.

In addition to these generalizations, there are a number of precautions that should be taken. While the MEP measurements can still provide a measure of cortical viability following a stroke, comparisons between the two hemispheres should be avoided (i.e., data from the non-affected hemisphere tells one nothing as to the current distributions in the peri-lesional region). Predictions of the precise targeting of focal cortical regions based on the surface coil position should not be made if the coil is placed proximal to the infarction site. Finally, comparisons between cortical and sub-cortical strokes should be viewed skeptically if the region of stimulation is proximal to the cortical or sub-cortical lesion (even though sub-cortical strokes have not been presented in this study, this author believes that alterations of the current density distributions will occur if the infarctions are not too distal from the cortical surface).

The stimulating induced current density distributions are clearly altered following a cerebrovascular event that damages the cortex in such a way that the tissue geometry and conductive properties are modified. Even though we focused on the perturbations caused by strokes in this paper, similar changes will be seen for any pathology that alters the cortical anatomy or the electric tissue properties. For example, CSF fills the void caused by the cerebral atrophy, much like is seen in stroke, and thus TMS induced stimulating currents could be altered in atrophic cortices as predicted by these stroke models (dependent upon the degree and location of atrophy). It is also possible that large sulci could cause similar current perturbations in healthy individuals; however, further study needs to be conducted in this area. The influx of CSF is not the only type of disturbance that will cause such current perturbations. For example, the conductivity of tissue is altered in the presence of a tumor and, thus, one could see current density perturbations if the tumor were found in the region of stimulation. In actuality, in the presence of any pathology, one cannot accurately predict the site, magnitude, and effects of TMS based on healthy head models.

In the case where displacement currents were included (i.e., the relative permittivity scheme was adjusted to 10⁷εₒ), the location of the current density maximum was still altered to essentially the same location in the models tested as predicted without the inclusion of displacement currents. The degree of perturbation was of the same order of magnitude as that seen without displacement currents, thus the overall percentage change was lower. However, the same trends and effects discussed without the inclusion of displacement currents still appear with their inclusion. Thus as the same perturbations and trends occur with the inclusion of displacement currents (and as such the inclusion of displacement currents will not change the predicted site or degree of stimulation because the process is still primarily conductive), their inclusion ultimately will need to be discussed on how displacement currents influence cellular dynamics.

As discussed in the foregoing, TMS effects may not be satisfactorily predicted in stroke patients by conventional methods based on healthy head models, when TMS is applied to damaged areas. The kind of perturbations observed in the stroke models will occur in other pathological cases in which the geometry or electrical characteristics of brain tissue are altered. These cortical current density perturbations could prove to be dangerous or at the very least lead to unreliable, erroneous results if guided by models that do not account for the electromagnetic tissue interactions. However, we have shown that the currents in the contralateral hemisphere are not affected (except when the coil is placed at the hemisphere boundary for a hemispherectomy), and as such the contralateral hemisphere makes for an ideal candidate for stimulation.

Recent studies suggest that invasive cortical brain stimulation is a useful therapy for stroke recovery. The development of transcranial magnetic stimulation (TMS) offers a non-invasive and painless alternative to stimulate the human cerebral cortex in conscious subjects. Therefore, repetitive TMS may be useful to modulate brain activity after stroke and enhance stroke recovery non-invasively. Due to interhemispheric interaction, we hypothesize that a possible target for rTMS is the contralateral undamaged motor cortex, the suppression of which by slow rTMS may release inhibition of the damaged hemisphere and promote recovery. After stroke, the nonlesioned hemisphere is disinhibited, perhaps due to the reduction in the transcallosal inhibition from the stroke-damaged hemisphere. This in turn may increase inhibition of the lesioned hemisphere by the disinhibited, unaffected hemisphere and could impair functional recovery. We report the results of a cross-over, sham stimulation-controlled, double-blinded study assessing the effects of modulation of the unaffected motor cortex by slow rTMS in patients within 12 months of a stroke.

The study included 10 stroke patients (3 men and 7 women) aged 37 to 73 years (mean 53.3 years) within 12 months of a stroke and six healthy controls (3 men and 3 women) aged 28 to 52 years (mean of 43.6 years). Written informed consent was obtained from all participants prior to inclusion in the study, which was approved by the local ethics committee.

Participants received three sessions of rTMS (1 Hz, 100% of motor threshold, 600 pulses) to the unaffected hemisphere over the primary motor (real or sham rTMS) and over the premotor cortex (real rTMS). The order of these different rTMS sessions was randomized and counter-balanced across participants. The different rTMS sessions were separated by one hour to minimize carry-over effects. Stimulation was delivered using a Magstim Super Rapid Stimulator equipped with a commercially available 8-shaped coil. For the sham stimulation, we implemented the same stimulation parameters used for the motor cortex stimulation (location and rTMS pulse train properties), but used a sham coil (Magstim Inc.).

All participants underwent a battery of the following tests to evaluate the motor function of the affected hand: 1) Simple Reaction Time; 2) 4-Choice Reaction Time; 3) Purdue Pegboard Test; 4) Finger Tapping. Each patient was tested at baseline and after sham, motor, and premotor rTMS. At baseline testing, participants were carefully evaluated regarding their ability to perform the required tasks, and they were allowed to practice until performance was stable. At this stage two patients were excluded because they showed very prominent co-contractions and proximal movements. The healthy controls were also tested at three time points each separated by one hour, but they did not receive TMS. The effects of rTMS to motor or premotor cortex on ipsilateral motor function have been previously investigated at our facility and were not the focus of the present study.

Our analysis was primarily focused on changes in sRT, cRT, Purdue Pegboard Test, and Finger Tapping performance. We used repeated measures of analysis of variance to test whether there was an overall effect of rTMS type (condition). When appropriate, post-hoc comparisons were carried out using Fisher LSD correction for multiple comparisons. Significance was set at p-value <0.05. Table 7 summarizes patients' demographics and stroke characteristics.

| | Age | | Classification of ischemic | | |
|---|---|---|---|---|---|
| Pt | (y) | Past medical history | stroke (Toast criteria)** | Localization of stroke | Neurological deficits |
| Pt 1 | 51 | HTN, NIDDM | Cardioembolic | Right frontal | Mild left hemiparesis |
| Pt 2 | 61 | HTN, NIDDM | Small vessel | Right internal capsule | Mild left hemiparesis |
| Pt 3 | 35 | HTN | Small vessel | Left corona radiata | Mild right hemiparesis |
| Pt 4 | 63 | HTN, NIDDM | Small vessel | Left internal capsule | Moderate right hemiparesis |
| Pt 5 | 55 | HTN, Tob | Small vessel | Right corona radiata | Mild left hemiparesis |
| Pt 6 | 57 | NIDDM, HLP, Tob | Small vessel | Right internal capsule | Moderate left hemiparesis |
| Pt 7 | 43 | HTN, Tob | Small vessel | Left internal capsule | Subtle right hemiparesis |
| Pt 8 | 58 | HTN, NIDDM, HLP | Large art/atherosclerosis | Left frontal | Moderate right hemiparesis |
| Pt 9* | 37 | HTN, HLP, Tob | Cardioembolic | Right frontal | Left hemiparesis and spasticity |
| Pt 10* | 73 | HTN, NIDDM | Small vessel | Right internal capsule | Severe left hemiparesis |

In Table 7, abbreviations are as follows: HPN—Hypertension; HLP—Hyperlipoproteinemia; Tob—Tobacco use; NIDDM—Non-insulin dependent diabetes mellitus. *Patients 9 and 10 were excluded from this study as they could not perform the motor tasks adequately. Repeated measures ANOVA showed that there was a main effect of condition on sRT (p=0.043) and cRT (p=0.045). Post-hoc comparisons demonstrated a significant decrease in sRT (p=0.014) and cRT (p=0.013) after real motor rTMS when compared to sham rTMS. Subjects also tended to be faster after real premotor compared to sham rTMS, however this result did not reach significance.

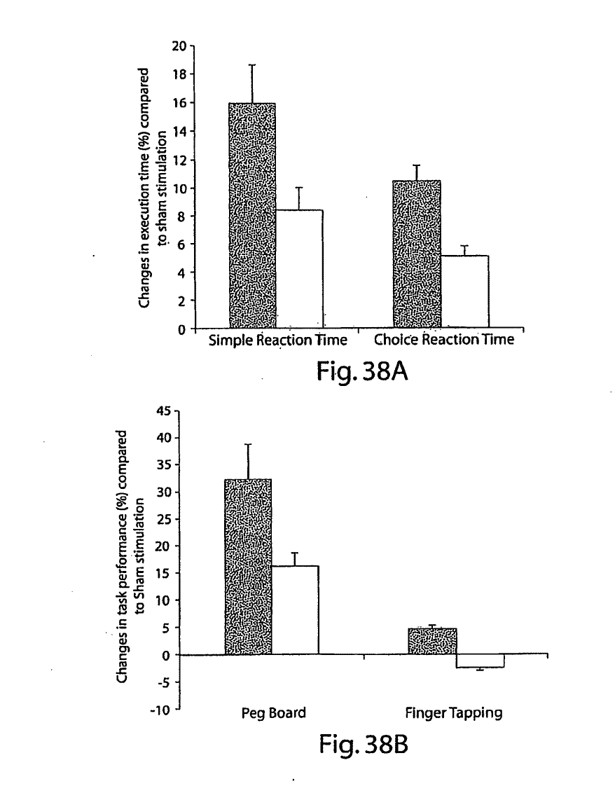

FIGS. 38A and 38B illustrate the effects of repetitive transcranial magnetic stimulation of the ipsilateral motor primary cortex and premotor cortex on motor tasks performance (sRT, cRT, Pegboard and finger tapping) compared to sham stimulation. A—Execution time after repetitive transcranial magnetic stimulation (rTMS). Change (%) in sRT and cRT after rTMS of motor (black column) and premotor cortex (white column) compared to sham stimulation. The execution times (sRT and cRT) were significantly shorter only after rTMS of primary motor area. B—Pegboard and finger tapping performance after repetitive transcranial magnetic stimulation (rTMS). Change (%) in Pegboard and finger tapping performance after rTMS of motor (black column) and premotor cortex (white column) compared to sham stimulation. There was significant increase in the number of correctly placed pegs after real motor rTMS compared to sham stimulation. There was no significant effect of TMS in the finger-tapping performance after stimulation of either motor or premotor cortex. Each column represents mean performance on the task SEM.

The Pegboard test results behaved in a similar manner as the reaction time tests, although three patients couldn't perform this task due to proximal arm weakness. Repeated measures ANOVA demonstrated a main effect of rTMS condition (p=0.006). Post-hoc comparisons revealed a significant increase in the number of correctly placed pegs after real motor rTMS (6.2±2.9) as compared to sham stimulation (4.2±2.4; p=0.002). The effects of rTMS onto premotor cortex did not reach significance (FIG. 38B). Because of the small number of patients (5 patients), a non-parametric approach was tested to validate our results. Wilcoxon Signed Rank demonstrated a significant increase in the number of correctly placed pegs after real motor rTMS as compared to sham stimulation (p=0:043).

However for the finger tapping test, repeated measures of ANOVA showed that there was no main effect of the rTMS condition on the finger-tapping test (F=0.27, DF=7.2 p=0.76). Although, performance tended to be better after real motor rTMS when compared to sham stimulation, this effect was small (less than 5%) and variable across patients (FIG. 38B).

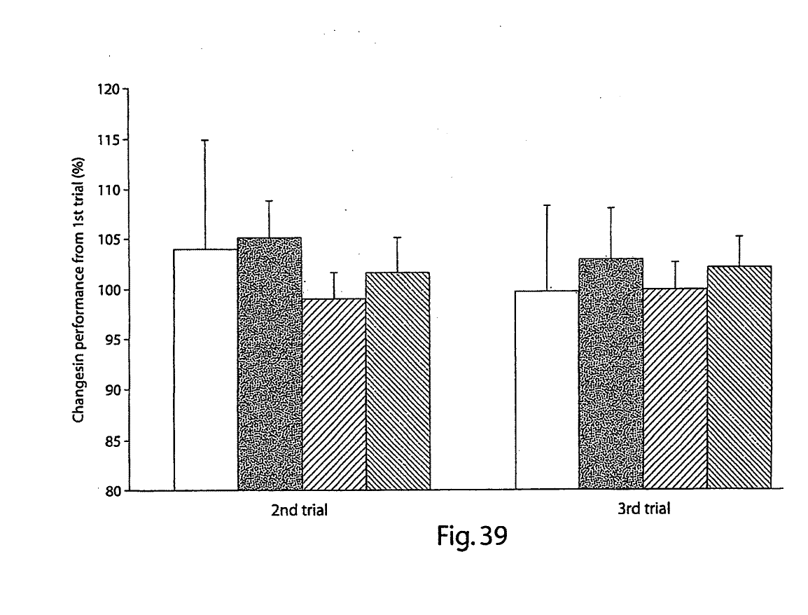

In the control experiment, healthy participants did not show changes in any of the four tests across repeated testing, as illustrated in FIG. 39 which shows that healthy participants did not show significant changes in any of the four tests (sRT—white column-, cRT—black column-, Purdue Pegboard—dark gray column-, finger tapping—clear gray column) across repeated testing. Each column represents mean performance on the task SEM. Repeated measures of ANOVA showed that there was no main effect of the testing condition for any of our tests.

Applicant's results are consistent with similar studies in healthy subjects and patients with non-motor strokes. Repetitive TMS (1 Hz, 90% MT) applied for 10 minutes to the motor cortex of 16 healthy subjects resulted in a shortening of execution time of an overlearned motor task with the ipsilateral hand. Furthermore, improvement of behavior by disruption of activity in the undamaged hemisphere with 1 Hz rTMS has also been demonstrated in patients with non-motor strokes, such as patients with neglect after parietal lesions and patients with non-fluent aphasia after a left hemispheric frontal stroke.

However, a similar study of 5 stroke patients showed no improvement in the motor function of the paretic hand after 1 Hz rTMS of the undamaged hemisphere. The different results may be due to patient selection—patients with chronic stroke (older than 1 year of stroke)—and task evaluation—only finger tapping. In our study, our patients were studied within the first year after their stroke and we also failed to find significant changes in finger tapping test. Indeed, a lack of significant effects observed after ipsilateral stimulation on finger tapping test has been previously described and might be the result of task specific variables, small sample size, or a ceiling effect. As has been demonstrated in this study, rTMS could make a potentially powerful tool for neurorehabilitation.

Since its inception, researchers have proposed the use of TMS and rTMS to study and treat neuropsychiatric diseases, such as major depression, schizophrenia, Parkinson's disease, dystonia, epilepsy and stroke. However, a fundamental question that needs to be addressed before wide-spread use of TMS in clinical practice is whether the modification of brain anatomy and tissue properties caused by certain neuropsychiatric diseases can alter the effects of TMS.

With TMS, changes in the tissue anatomy and electromagnetic properties have been shown to alter the TMS induced stimulating currents in both phantom and modeling studies. We have shown previously that the damaged areas in patients with stroke can perturb the location and magnitude of the stimulating cortical currents. The main reason for this perturbation is that the altered distribution of cerebral spinal fluid (CSF) to brain tissue modifies the conductive tissue properties in the infarction region and effectively provides a path of lowered resistance for the stimulating currents to flow along. Several diseases explored with TMS, such as depression, Alzheimer's disease, Huntington's disease, corticobasal degeneration and Creutzfeldt-Jakob's disease, as well as normal aging, show related anatomical changes that could have an impact on the TMS induced electric currents. All of these populations are characterized by varying degrees of cortical atrophy, where brain tissue is replaced by CSF, the CSF to cortical tissue volume ratios increase, and oftentimes the cerebral sulci widen. However to date, there has been no systematic study of the effects that cortical atrophy and the altered CSF to cortical tissue ratios can have on the currents induced by TMS in neural tissue.

Herein we explore how the electrical and anatomical changes caused by cortical atrophy can perturb the TMS induced stimulating currents in the cortex through multiple MRI derived finite element TMS head models. We also discuss the possible clinical implications of the perturbations to the current induced under TMS in patients with cortical atrophy.

Multiple MRI based finite element head models were constructed and evaluated for various stimulation orientations to address the effects that cortical atrophy can have on perturbing the TMS induced stimulating current. An initial sinusoidal steady state finite element model (FEM) was developed using the Ansoft 3D Field Simulator software package with the eddy current solver. An MRI guided three-dimensional CAD rendering of the human head was used to solve for the currents induced in the cortex during magnetic stimulation, referred to as the healthy head model.

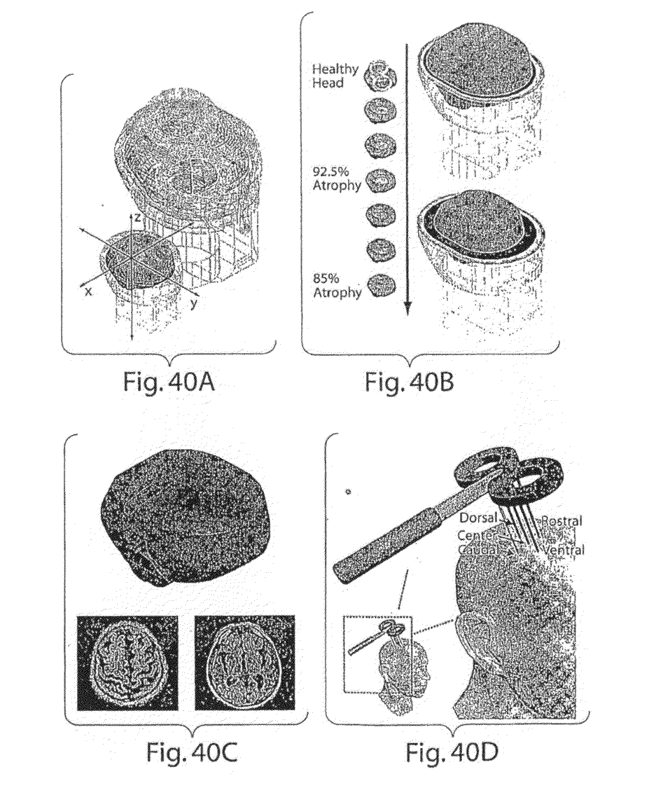

FIG. 40 illustrates the healthy head model and the model coordinate system (here shown in the healthy head model inset). The same coordinate system was used for all the models, note that the current density magnitude solution is shown for reference where the solution is that of the healthy head model in the DLPC coil position. FIG. 40, plot B illustrates increasing symmetric atrophy models: The models are displayed from the healthy head model to the 85% atrophy model, on the left side highlighting the size of the brains. On the right side the healthy head model (upper) and the 85% atrophy model (lower) are shown to highlight the increasing thickness of the CSF and the decreasing cortical thickness. Notice the increasing CSF thickness and the decreased cortical size between the two models.

FIG. 40, plot C illustrates the widened sulci model: The base sulci model is shown with the widened sulcal regions highlighted and sample MRI slices of the model are included. FIG. 40, plot D illustrates evaluation line locations: The current density magnitudes were calculated along 5 different evaluation lines which were inserted through the head model. The lines were located relative to the coil; with the center line directly located at the figure-of-eight coil center and normal to the coil face and the other lines 1 cm ventral, dorsal, anterior, and posterior to the center line (note that the figure is not drawn to scale but with the lines and the coil drawn to highlight their placement).

This model was generated to include the skin, skull, CSF, gray matter, and white matter. The tissue conductivities in the model were assigned the mean value from multiple references; skin at 0.465 S/m, bone at 0.010 S/m, CSF at 1.654 S/m, gray matter at 0.276 S/m, and white matter at 0.126 S/m; thus, each individual tetrahedra of the model was assigned the conductivity corresponding to its tissue type for the FEM calculations. The tissue permittivities were considered to have a negligible effect on the primary focus of this study and assigned the 10⁴εₒ scheme for the bulk of the studies.

The source was modeled as a figure-of-eight coil with two 3.5 cm radius windings made of a single turn of 7 mm radius copper wire. The copper was modeled as a perfect conductor (currents constrained to the surface) with the permittivity set to (8.854×10⁻¹² F/m), conductivity of 5.8×10⁷ S/m, and a permeability of 1.0μₒ (4π×10⁻⁷ H/m). The source current was set at 5 kHz with a 1.8×10³ A peak current (5.65×10⁷ A/s, rate of change of the peak current with time). For each solution obtained, the coil was positioned with its facial plane tangential to the scalp and 7 mm above the surface to account for the insulating layer found in commercially available coils. The x, y, z coordinate system used is defined in FIG. 40, plot A and uses mm units.

Ten different atrophy models of increasing degrees of cortical atrophy and varied cortical modifications were used to compare to the healthy head model under different stimulation conditions. Six of the models were constructed by decreasing the overall volume of the gray and white matter, while increasing the overall CSF volume in the healthy head model, as is seen in in-vivo imaging and postmortem histology studies analyzing anatomical atrophic changes across the human lifespan. This was done by decreasing the volume of the healthy head model's brain tissue symmetrically by 2.5% steps, from 100% to 85%, and filling the area with CSF. These models will be referred to by their percent of atrophy (i.e., the 97.5% atrophy model will refer to the model with a brain volume of 97.5% of that of the healthy head model). See FIG. 40, plot B for a graphical representation of the models.

Four additional models were constructed to explore the effects of expanded cerebral sulci. These models were constructed by altering the healthy head model's cortical geometry based on the MRIs of patients treated at Beth Israel Deaconess Medical Center (but with the atrophic regions cut with straight edges along their borders and with tissue regions clearly demarcated for use in the FEM solver). One model was constructed by removing the cortical tissue in the areas of the central sulcus and the sylvian fissure from the healthy head model and replacing the gray matter with CSF. This model is referred to as the base sulci model. Three further models were constructed based on this base sulci model, but with symmetrically decreased cortical volumes, referred to as the 95%, 90%, and 85% sulci models (with 95%, 90%, and 85% of the brain volume of the base sulci model). See FIG. 40, plot C for the base sulci model and its MRI basis.

In order to investigate the effects that increasing atrophy can have on the induced stimulating currents, solutions were obtained and contrasted for both the healthy head model and the six symmetric atrophy models with the coil placed over the right dorsal lateral prefrontal cortex and the right motor cortex (with the coil center located at (43.8,51.0,48.0) and (43.1,7.5,51.0) respectively).

For each model and coil position, the magnitude and location of the maximum cortical current density were evaluated. Secondarily, the surface area on the cortex where the current density was greater than 90% of its maximum value was calculated. We will refer to this as the maximum cortical current surface area. Then, to examine the current behavior in the region of the expected current density maximum, the current density magnitude was determined along evaluation lines perpendicular to the coil at its center and at locations 10 mm anterior, posterior, rostral, and caudal to the center line (as defined in FIG. 40, plot D). The distance from the skin surface to the cortical surface along each of these lines was determined for each model. Finally, the induced current density vector behavior was also analyzed in all the tissues.

Once the magnitude of the current density at the cortical interface and the distance from the scalp to the interface were determined for each model, plots were generated of the maximum magnitude on the surface of the cortex of each model, relative to the healthy head model, as a function of distance into the brain model along the aforementioned evaluation lines. We constructed a set of stochastic models of the relative magnitude of the cortical current density as a function of distance from the coil. In one embodiment, the current magnitude was modeled using a single decaying exponential function with Gaussian noise drawn from a single distribution, irrespective of coil location (i.e., this model accounted for scalp to cortex distance alone and was represented by a single exponential function).

An alternative model postulated that the current magnitude was described by separate distributions for each coil location, each with distinct exponential decay functions and noise variance parameters (i.e., this model accounted for both scalp-to-cortex distance and the coil position and was represented by two separate exponential functions, one for the DLPC and one for the motor strip coil locations—note that the coil position relative to the underlying electrical/anatomical distribution were constant for each line in this model embodiment). The exponential and variance parameters for the above models were fit with maximum likelihood methods. Using these models, we constructed a likelihood ratio test for the null hypothesis that the data from both coil positions was captured by the single exponential function versus the alternative hypothesis that the data was better explained by a distinct exponential function for each position.

Solutions were obtained with the coil placed above the dorsal lateral prefrontal cortex for the four widened sulci models and compared to the heterogeneous healthy head model and the analogous atrophy models with the coil in the same position. The analysis of the magnitude and location of the maximum cortical current density, the maximum cortical current surface area, the current density magnitude behavior along the evaluation lines, and the induced current density vector behavior detailed in the section above was completed for these models.

Every model converged below the 1.0% energy error stopping criteria defined in the foregoing. The average number of tetrahedra for the atrophy model solutions was 131,258 and on average 1,260,551 kbytes of memory were used by the solver for each model solution. Multiple machines, of varied computing resources, were used in the solution process; however, typical convergence times for a dual 3 GHz Xeon processor machine with 4 Gigs of Ram were as follows: 3:31:06 for the healthy head model with the coil in the DLPC position (140,509 tetrahedra and 1304804 Kbytes of memory used) and 3 hours 18 minutes and 3 seconds for the 85% atrophy model with the coil in the motor strip location (132,383 tetrahedra and 1,195,452 Kbytes).

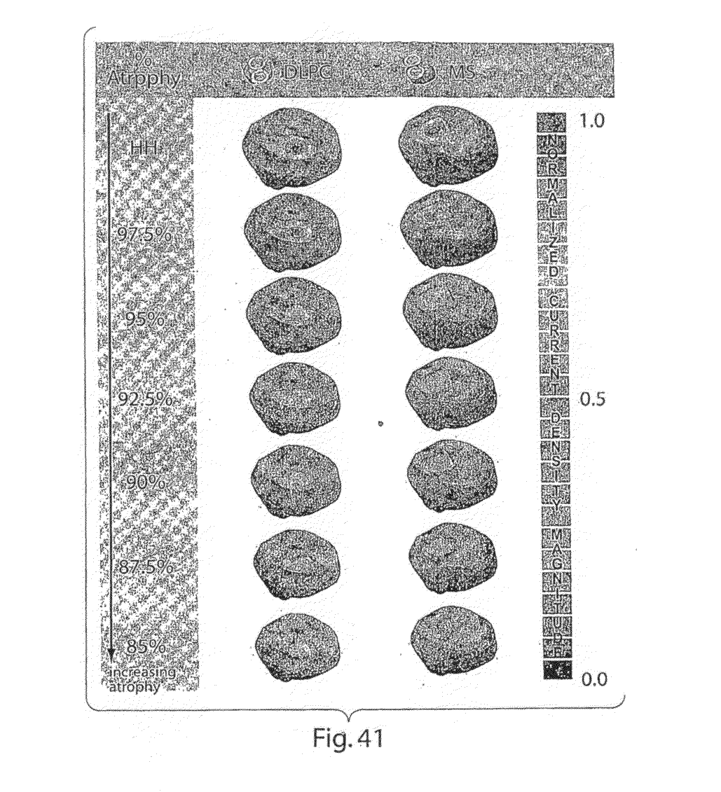

FIG. 41 illustrates current density magnitudes: the current density magnitudes are plotted for the dorsal lateral prefrontal cortex (DLPC) and the motor strip (MS) coil position (the coil position is shown as a reference on the top text row in the healthy head model for each placement). The plots are included from the healthy head model (HH) to the 85% atrophy model (from top to bottom). Note that the normalized current density magnitude is relative to the maximum current density magnitude for the healthy head model for each coil position (i.e., the DLPC scale ranges from 0 to 2.82 A/m² and the MS scale ranges from 0 to 3.57 A/m².

With the coil placed over the motor strip, the maximum cortical current densities ranged from 3.57 to 2.36 A/m² from the healthy head to the 85% atrophy model respectively, decaying by approximately 33.9% at a vertical distance of 12.4 mm from the scalp (See FIG. 41). The location of the maximum cortical current density was found at increasingly anterior and medial positions relative to the coil with increasingly severe atrophy, i.e., models with higher atrophy showed the larger shift in location of the maximum cortical current density. The maximum cortical current surface areas ranged from 191 to 238 mm² and had no consistent trends in area relative to the degree of atrophy.

With the coil placed over the dorsal lateral prefrontal cortex, the maximum cortical current densities ranged from 2.82 to 1.88 A/m² from the healthy head to the 85% atrophy model respectively, decaying by approximately 33.3% at a vertical distance of 14.5 mm from the scalp (See FIG. 41). The location of the maximum cortical current density was found at the CSF-gray matter interface with little lateral variation relative to the coil as the degree of atrophy was increased. The maximum cortical current surface areas ranged from 143 to 224 mm², again with no consistent trends in area relative to the degree of atrophy. The magnitude, location (and vertical distance from the scalp), and area of the maximum cortical current densities are tabulated for the various models in Table 8 below.

TABLE 8

| Model& | | % HH | | VerD | |
|---|---|---|---|---|---|
| Position | MCDCS | CDMax | MCDCS Location | scalp | Area |
| HHms | 3.57 | 100.0 | (29.8, 7.2, 35.3) | 5.7 | 218.0 |
| 975ms | 3.20 | 89.6 | (30.5, 9.9, 33.9) | 6.9 | 228.0 |
| 95ms | 2.98 | 83.5 | (29.7, 7.6, 33) | 8.3 | 221.2 |
| 925ms | 2.78 | 77.9 | (31.6, 7.4, 31) | 9.3 | 238.0 |
| 90ms | 2.59 | 72.5 | (28.5, 7.9, 31.1) | 10.4 | 217.1 |
| 875ms | 2.45 | 68.6 | (25.9, 12.2, 31) | 11.4 | 191.2 |
| 85ms | 2.36 | 66.1 | (22.4, 12.9, 31.1) | 12.4 | 209.0 |
| HHdlpc | 2.82 | 100.0 | (34.5, 45, 29.6) | 6.5 | 224.0 |
| 975dlpc | 2.58 | 91.5 | (34.5, 42.3, 28.86) | 7.7 | 176.0 |
| 95dlpc | 2.37 | 84.0 | (33.7, 41.1, 28.1) | 8.9 | 218.0 |
| 925dlpc | 2.32 | 82.3 | (33, 43.7, 26.5) | 10.3 | 170.3 |
| 90dlpc | 2.27 | 80.5 | (32, 42.7, 25.8) | 11.5 | 143.0 |
| 875dlpc | 2.08 | 73.8 | (31.1, 41.8, 24.9) | 12.7 | 149.0 |
| 85dlpc | 1.88 | 66.7 | (32.4, 42.9, 22.7) | 14.5 | 192.4 |

Table 8 tabulates data for the Maximum Current Density on the cortical surface. The first column indicates the model and coil position; where HH stands for healthy head model, ms for the motor strip coil position, dlpc for the dorsal lateral prefrontal cortex coil position, and the number corresponds to the degree of atrophy (i.e., 925 ms indicates the 92.5 atrophy model with the coil in motor strip position. The second column indicates the maximum current density on the cortical surface (MCDCS), in A/m². The third column indicates the magnitude of the current density on the cortical surface relative to the healthy head model for the coil in the same position. The fourth column provides the MCDCS location, while the fifth column indicates the vertical distance from the scalp to the MCDCS location. And the final column reports the maximum cortical current surface area in mm².

For both the motor and prefrontal coil positions, the induced current density vector behavior in the tissues was consistent in behavior with those of previous studies where the vector orientation followed a figure-eight path with the greatest irregularity at the tissue boundaries and with the current density vectors aligned in the region of the current density maximum.

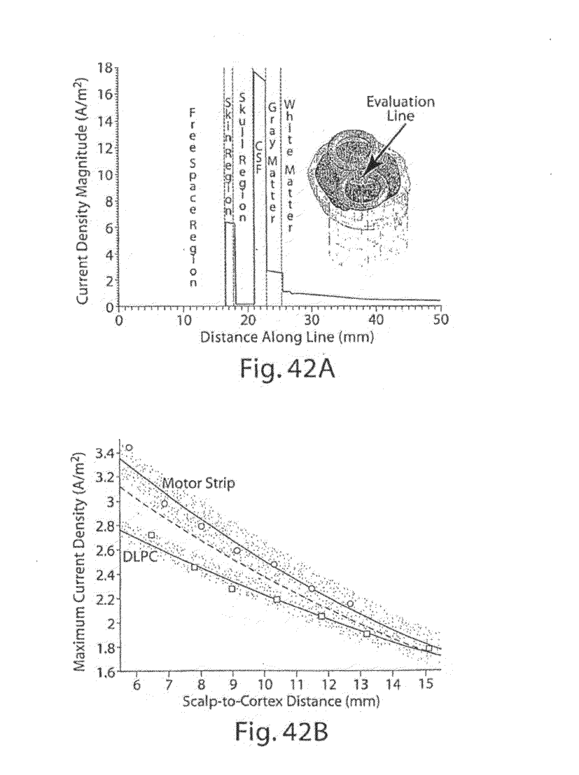

The current density magnitude was calculated along the evaluation lines that were defined in FIG. 40, plot D. The current density showed stair step jumps in magnitude at the tissue boundary interfaces in every solution, which correlated to the conductivity of the tissues (see FIG. 42 for graphical examples of the cortical current density behavior along the center evaluation line for the healthy head model). With the coil placed over the motor strip, the current density magnitude ranged from 3.43 to 2.12 A/m² (for the healthy head to the 85% atrophy model) at the location where the center line intersected the surface of the cortex. This represented a 38.2% decay from the value calculated in the healthy head model along the center line 12.7 mm from the scalp to the point of intersection on the cortical surface in the 85% atrophy model. The location of intersection was located from 6.3 to 16.2 mm distant from the actual location of the maximum cortical current density in each of the individual models, but always within the maximum cortical current surface areas.

These results and those calculated for the coil placed over the dorsal lateral prefrontal cortex are tabulated in Table 9 below. Table 9 includes data for the Maximum Current Density on Center Evaluation Line. The first column indicates the model and coil position. The second column indicates the maximum current density on the cortical surface where the center line intersects the cortex, in A/m². The third column indicates the magnitude of the current density at the cortical surface intersection relative to the healthy head model for the coil in the same position. The fourth column indicates the distance from the scalp to the cortical intersection along the evaluation line.

TABLE 9

| Model& | CLine | % HH | LineDis |
|---|---|---|---|
| Position | CSCD | CDMAx | from scalp |
| HHms | 3.43 | 100.00 | 5.8 |
| 975 ms | 2.96 | 86.30 | 6.9 |
| 95 ms | 2.77 | 80.76 | 8.0 |
| 925 ms | 2.57 | 74.93 | 9.2 |
| 90 ms | 2.45 | 71.43 | 10.4 |
| 875 ms | 2.25 | 65.60 | 11.5 |
| 85 ms | 2.12 | 61.81 | 12.7 |
| HHdlpc | 2.70 | 100.00 | 6.5 |
| 975dlpc | 2.43 | 90.00 | 7.8 |
| 95dlpc | 2.25 | 83.33 | 9.0 |
| 925dlpc | 2.16 | 80.00 | 10.4 |
| 90dlpc | 2.02 | 74.81 | 11.8 |
| 875dlpc | 1.87 | 69.26 | 13.2 |
| 85dlpc | 1.74 | 64.44 | 15.1 |

Similar trends were calculated along the other lines for both the motor strip and dorsal lateral prefrontal cortex coil positions; with a greater decay where the lines ran proximal to the lateral cortical face such that they intersected at deeper points. The results for the Anterior, Posterior, Ventral, and Dorsal lines are tabulated in supplementary Tables 6.1S-6.44S, shown in the Appendix of the '847 patent. Additionally, this analysis was completed for other locations in the model to ascertain whether the effects were confined to the CSF-gray matter interface; in Table 6.5S the results are reported for locations 1 mm below the gray matter-white matter interface along the center line.

Once the magnitude of the current density at the cortical interface and the distance from the scalp to the interface were determined for each model, plots were generated of the maximum magnitude on the surface of the cortex of each model relative to the healthy head model. FIG. 42 illustrates current density behavior with distance. FIG. 42, plot A illustrates current density magnitude evaluated along the center evaluation line in the healthy head model with the coil in the dorsal lateral prefrontal cortex coil location. Note that the current density magnitude varies with the conductivity of the tissues. The inset shows mesh model with the current density magnitude plotted on the surface of the cortex with the center evaluation line shown intersecting the tissues (center line is in black and circled by the blue dashed line.

FIG. 42, plot B illustrates exponential models for maximum current density: exponential models for maximum cortical current density as a function of distance for position dependent and independent models. Circles and squares represent simulated current density values from the motor strip and the dorsal lateral prefrontal cortex coil positions respectively. The dashed line represents the mean exponential trend for the position independent model. The light green and light blue areas represent a 95% confidence region for the position dependent models. Note that in the position dependent models the position was kept constant for each exponential function (and thus the coil location and the coil angle relative to the electrical/anatomical tissue distribution were kept constant).

Exponential models for the magnitude with Gaussian errors were fit to the data series as a function of either scalp to cortex distance alone or for both scalp to cortex distance and coil position using maximum likelihood. The expected values of these models are shown as trend lines in FIG. 6.3B for both models (the model with one line accounted for the scalp to cortex distance alone and the second model with two lines, one for the DLPC and one for the motor strip, accounted for both the coil position and the scalp to cortex distance). If the relationship between distance and current magnitude were independent of coil position we would expect the exponential curves to coincide. However, the parameters of these exponential models were found to be significantly different, suggesting that distance alone does not predict current magnitude as well as distance and coil position considered together.

The null hypothesis that the data from both coil locations came from the model of distance alone against the alternative hypothesis of the expanded model using a log likelihood test was tested, and found that the data strongly rejected the null hypothesis (p<1E-15). The alternative model with separate distributions for the two coil positions explains over 97% of the variance in the cortical current density (i.e., this is to say the model which accounted for both the coil position and scalp to cortex distance with 2 distinct lines, one for each coil position tested, which kept the coil position and tissue distribution unique and constant for each line, accounted for 97% of the variability).

The model which accounted for just scalp to cortex distance alone failed to account for the variables (coil location and angle relative to the underlying tissue distribution) and as such could lead to significant errors. Similar results could be found for analysis along the different lines and at different locations in the model (e.g., 1 mm below the CSF-gray matter interface, at the gray matter-white matter interface, 1 mm below the gray matter-white matter interface, etc.).

In the widened sulci models, there was a less consistent behavior in the location of the current density maxima. The behavior of the current density distribution directly under the coil center was very similar to the analogous atrophy models, but far less predictable along the widened sulci borders, similar to the results seen in stroke studies and heterogeneity studies. The current density maximums found directly below the coil center ranged from 2.95 to 1.97 A/m² (and were consistent with the results of the analogous atrophy models without the widened sulci).

However, for the base, 95%, 90%, and 85% widened sulci models, there were locations along the sulci borders where current density magnitudes were within +/−15% of the maximums found under the coil center (3.59%, −0.85%, 6.61%, and 14.35% respectively) and were well above 150% of the current density magnitude (i.e., increased by over 50%) in the exact position in the analogous atrophy models without the widened sulci. These current density maximums were found directly below the most posterior portion of the figure-of-eight coil on the border of the widened central sulci, with maximum values ranging from 2.05-3.06 A/m².

The maximum cortical current surface areas along the atrophic sulci borders were very focused, all less than 10 mm². In general the current density was increased in regions proximal to the widened sulci (within approximately 1 cm), most particularly in the region of the central sulcus that was proximal to the posterior portion of the figure of eight coil. FIG. 42 illustrates widened sulci current density variations. FIG. 42, plot A illustrates current density magnitudes: the current density magnitudes are shown for the base and 90% widened sulci models with particular focus on the region near the central sulcus, with the same locations highlighted for the analogous models without the widened sulci in the cortex (i.e., healthy head and 90% atrophy models). Note that the current density magnitudes in the region of the figure-of-eight coil center's, indicated by the X's, are consistent for the models with the same % of atrophy.

However, the current density magnitudes increase as one gets closer to the widened central sulcus in each of the models in comparison to the models without the widened sulci; the black, blue, and red circles indicate analogous points in the models with consistent % atrophies at points 10 mm, 5 mm, and 2.5 mm respectively from the widened sulci and the current density magnitudes are indicated in A/m². Note that the current density scale is normalized to the maximum current densities for the widened sulci models (i.e., the models on the left are normalized to the maximum cortical current density in the base widened sulci models, 3.06 A/m², and for the 90% widened sulci model, 2.42 A/m².

FIG. 42, plot B illustrates current density vector distribution: The left most image shows the vector density distribution on the surface of the cortex for the healthy head and base widened sulci model highlighting the behavior in the central sulcus region. The right most image shows the vector behavior on the surface of the CSF for the healthy head and base widened sulci model, note that the current density vectors point into the region of the widened sulci.

The differences in magnitude were generally unchanged at a distance greater than a centimeter away from the borders, but increased with decreasing distances from the border (for example in FIG. 42, plot A, note that at the points evaluated the current density differences increase by 7.6% to 19.1% to 24.7% to 32.9% at distance of 10 to 5 to 2.5 to 1 mm away from the widened central sulcus in the base model). There was less of a change in the current densities along the widened Sylvian fissure, where the differences in current density magnitudes were generally less than +/−10% of the magnitude of the current density at the same location in the analogous models without the widened sulci. In general the degree of change was greater as the degree of atrophy increased. These results are tabulated in Table 10.

TABLE 10

| Model& | MCDCS | MCDCS Location | VerD | Area |
|---|---|---|---|---|
| Position | under coil | under coil | scalp | (mm²) |
| Bws | 2.93 | (32.9, 43.9, 28.1) | 6.9 | 168.0 |
| 95ws | 2.38 | (34, 41.2, 28.0) | 8.9 | 272.0 |
| 90ws | 2.26 | (37.9, 34.6, 25.3) | 11.5 | 111.0 |
| 85ws | 1.97 | (37.8, 35.6, 22.1) | 14.6 | 78.0 |
| | MCDCS | MCDCS Location | VerD | |
| | on sulcal edges | on sulcal edges | scalp | Area |
| Bws | 3.06 | (45.2, 4.1, 28.1) | 6.8 | 5.1 |
| 95ws | 2.44 | (25.3, −2.2, 34.6) | 7.9 | 4.5 |
| 90ws | 2.42 | (24.2, −3.8, 32.6) | 10.2 | 2.2 |

Note:
two other locations were within +/− 15% of the under the coil max: at
(41.4, 1.83, 24.7) with 2.37 magnitude & 3 mm² area and at
(45.2, −0.1, 20.4) with a 2.38 and 4 mm² area

| | | | | |
|---|---|---|---|---|
| 85ws | 2.30 | (36.4, 2.06, 24.17) | 12.5 | 9 |

Note:
three other locations were within +/− 15% of the under the coil max:
At (24.3, −5.3, 30.2) with a 2.05 mag and 4 mm² area, at
(30.4, −7.2, 20.7) with a 2.21 mag and 6 mm² area and at at
(42.5, −0.4, 19.2) with a 2.09 mag and a 4 mm² area.

Table 10 includes data for the maximum current density on the cortical surface for the widened sulci models. The first column indicates the model and coil position; where Bws stands for base widened sulci model, ws for widened sulci, and the number corresponds to the degree of atrophy (i.e., 85ws indicates the 85% widened sulci model. The second column indicates the maximum current density on the cortical surface (MCDCS), in A/m² both under the coil and along the sulcal border. The third column provides the MCDCS location under the coil and along the sulcal border, while the fourth column indicates the vertical distance from the scalp to the MCDCS location. And the final column reports the maximum cortical current surface area. Note that for the 90ws and 85 ws models, there were multiple regions along the border where the current density was with +/−15% of the maximum current density under the coil, these are reported in the adjacent row in the table.

The behavior of the current densities along the evaluation lines, and thus the current density behavior in the area of the coil hot spot in the analogous models without the widened sulci, was largely similar to those in the non-widened sulci models. See Table 6.6 Supplementary in the Appendix of the '847 application for the evaluation line data.

The current density vector behavior was consistent in the area under the figure-eight-center with the other atrophy models, but altered along the sulci's borders and in the surrounding tissues. The current density vector distributions deviated from predictable figure-of-eight distributions that were seen in the cortices of the models without the widened sulci to conform to the sulcal boundaries, such that the current vectors became more perpendicular to the borders. Additionally, in the CSF the current density vectors were directed into the widened sulci in the CSF surrounding the sulci, see FIG. 42, plot B.

When one analyzed the current density behavior within the white matter along the widened sulcal boundaries similar effects were seen. The maximum current density magnitudes in the white matter of widened sulci models ranged from 105% to 130% of the maximums in the models without the widened sulci (from 1.14 to 0.76 A/m² along the gray matter-white matter interface in DLPC models without widened sulci as compared to 1.20 to 0.99 A/m² in the models with the widened sulci) and the widened sulci maximums were found from 5.3 to 4.9 cm from the maxima locations in the models without widened sulci.

Additionally, the surface areas of maximum current density in the white matter were very focal, and confined to areas of less than 10 mm². As within the gray matter, the differences in magnitude were generally unchanged at a distance greater than a centimeter away from the sulcal borders, but increased with decreasing distances from the border. Similar trend line functions were seen along the evaluation lines under the coil location removed from the sulcal boundaries.

Finally, the current density vector alterations that were seen in the gray matter and CSF due to alterations in the boundary of unequal conductivities are similarly seen in the white matter (note that this modular type of view-point is clearly used for descriptive purposes of the atrophy effects, and it should be clearly stated that the final current density distribution is dependent upon the entire inter-relationship of the physical boundary conditions constraining the distribution in an integrated fashion; i.e., ultimately the behavior of the current densities in one tissue can not be ignored in relation to that in another tissue because they are inter-related).

The results of Applicant's studies show that the disruption due to atrophy can modify the effect of TMS in several ways. As expected, the magnitude of the current densities induced on the cortex was dependent upon the degree of cortical atrophy and decreased in magnitude as the distance from the scalp to the cortex increased. However the degree of attenuation in the cortical current densities did not depend on just the distance alone, but also depended on the relative coil position and the anatomical and electrical distribution of the tissues. The location of the maximum cortical current density varied with the degree and type of atrophy, and the current density vector behavior was altered in the widened sulci models, conforming to the altered tissue geometries.

In addition to analyzing the current density results for the individual models, exponential trend lines were fit to the data to predict how the current densities would decay at different locations in the models. Along the individual evaluation lines which accounted for position and distance these models fit very well with the data; but, no single exponential model successfully captured the relation between cortical current density and the distance between the scalp and cortex along different evaluation lines. These concepts are discussed in detail below for each of the modeling schemes individually (Note that although we focus the analysis and discussion along the CSF-gray matter interface, the trends and conclusions analyzed herein are indicative of the current density alterations throughout atrophic brains at depths below the CSF-gray matter interface; i.e., deeper cortical layers and within the white matter). Additionally, the clinical implications and limits to the modeling studies are discussed.

The magnitude of the maximum cortical current density decreased with increasing scalp to cortex distances in each of the models. While maintaining the coil location at a constant position and evaluating the current density as a function of the distance, the degree of attenuation could be well predicted by exponential functions. Along a single evaluation line, these exponential models of distance alone explained over 97% of the variability in the cortical current magnitude, suggesting that the distance between the scalp and cortex is an important predictor of the expected degree of attenuation when TMS is used in the setting of cortical atrophy (i.e., with distinct exponential functions for the DLPC and motor strip, where the coil position and relative anatomical/electrical tissue distribution was kept constant for each function). However, no one single function based on the distance alone could predict the degree of attenuation for the coil placed at different locations on the scalp (or for a single coil position at different locations). Our data strongly indicate that the functional form of the current magnitude attenuation differs between the two coil positions studied, suggesting that other factors such as the relative coil to scalp location and the electrical properties of the tissues significantly affect the final current density distribution.

When we repeated these studies for other locations in the brain volume, such as at points 1 mm below the gray-white matter interface, the results were the same (i.e., no one single function based on the distance alone could predict the degree of attenuation for the coil placed at different locations on the scalp). In light of these findings, we conclude that the value of functions, based solely on static magnetic field measurements or simplified models, that have been proposed to adjust the source magnetic field strength based on the scalp to cortex distance are insufficient and should be reevaluated for the clinical use. Such functions provide an initial estimate of the source field attenuation, but completely ignore the tissue to field interactions, the effects of altered anatomy, and the relative coil position.

The location of the current density maximum was altered as the degree of atrophy was increased. The distance between the calculated current density magnitude and the location as predicted by the center line intersection generally increased as the degree of atrophy increased. This effect was more prominent when the coil was placed over the motor strip than over the dorsal lateral prefrontal cortex. This difference can be explained by the degree of the change in tissue geometry relative to the coil position for each model, whereby the relative distance from the most lateral (and directly caudal to the coil) curvature of the brain face was more pronounced as the atrophy increased with the coil in the motor strip position.

These results suggest that trackers which predict the site of stimulation based on the center line intersection can introduce inaccuracies in predicting the exact location of the current density maximum when changes in the head anatomy and the tissue electric properties in the coil region are ignored. However, for all of the models, the center line was located within the 1 mm of the maximum current density area (i.e., the region where the current density ranged from 90% to 100% of its maximum); and, although one might not be able to determine the exact location of the current density maximum with center line projection based frameless stereotactic tracking systems, these results suggest that the center line prediction is accurate enough to gauge the area where the current density magnitude is within 90-100% of its cortical maximum as long as there are not infarction sites, expanded sulci (see below), or similar pathologies which drastically alter the cortical geometry and conductive matrix of the tissues in the region of stimulation.

For the TMS frequency spectrum and the tissues studied, the current density vector distribution is governed by the boundary condition that the normal current density vector components must be continuous across boundaries of differing conductivities. Thus, when one goes from the more highly conductive CSF to the less conductive cerebral tissue at the widened sulci borders (or from the more conductive gray matter to the white matter), a jump in normal component of the electrical field is expected when compared to the healthy head model, or where the tissue was previously a homogenous medium. This will dictate the final current density direction and magnitude along the sulcal borders. Thus, the new small areas of maximum cortical current density magnitude found along the sulcal borders, in areas of less than 10 mm², are indicative of these changes in the current density distribution and indicative of areas of least resistance. However, the extremes seen in these widened sulci models (where current densities were seen in excess of 150% in the same position of that in the analogous atrophy models without the widened sulci) will most likely be rare in real life situations of atrophy because the linear edges used in the model for the sulcal borders will be more curvilinear in-vivo (for two out of the four widened sulci models the maximum cortical current density was found directly on the linear edge of the sulcal border, see FIG. 42, plot A).

However there were still large areas of perturbation removed from these areas of extreme perturbation where there was a consistent increase in the current density in the regions of the expanded sulci (see FIG. 42, plot A). For example in FIG. 42, plot A, one will notice an increase in the current density magnitudes as one approaches the sulci borders in relation to the current density magnitude in the analogous models without the widened borders (generally increasing in magnitude from 10 to 40% of the values found in the analogous atrophy models within 1 cm of their boundaries). These current amplifications were dependent upon the geometry of the widened sulci and are basically indicative of a region of lowered resistance. In addition to the changes in magnitude, the current density vector orientation was altered along the sulcal borders. As stated above, the normal components of the current density must be continuous across the CSF-gray matter and gray matter-white matter interfaces at the sulcal borders, which results in the alteration of the current density vector orientations along the border (see FIG. 42, plot B for an example). Additionally, a shift in the CSF currents was observed directed into the sulcal regions (see FIG. 42, plot B).

While herein we have completed the analysis with a comparison of models with widened sulci and those models without sulci included, we think that it is important to note many of the effects that we explain can occur in theory in normal sulcal regions to varying degrees. The exact effects will obviously depend on the geometry and tissue electrical properties in the regions of the sulci, but earlier studies have clearly shown the effects of heterogeneities can be significant and these heterogeneous conditions are clearly seen at sulcal borders. We feel that the effects will be most extensive at regions where the border geometries result in corner regions (such as could be seen along irregular edges of scar tissue along an infarction border) as is seen in studies examining corner points in other electromagnetic models.

Within this study, both symmetric atrophy and widened sulci models were examined. In the clinic, the demarcation in conditions will be less distinct and some combination of each will be present. However as the results displayed, with increasing atrophy and cortical modification, the current density distributions were altered in magnitude, orientation, and location; all of which will alter the population of neural elements stimulated and ultimately lead to practical and clinical implications.

Even though the scalp-brain distance alone does not account for the final current density distributions, it is clear that with increasing scalp to cortical distances precautions need to be taken into account with TMS. One should not use motor evoked potential (MEP) reference values between locations of varied scalp to cortex length to assess differences in network stimulation. In-vitro experimental and modeling data suggests that the site of activation is predicted by the peak electric field magnitude, and thus the cortical current density in the cortical neurons.

Additionally, in-vivo TMS experiments in both the motor and visual cortex have provided evidence that stimulation occurs at the location of the peak electric field. Thus, with minimal changes in the neural architecture (i.e., relative neural cell to current density orientations) the current density magnitude attenuation that is seen with increasing atrophy should lead to an expected alteration in the MEP values in atrophic regions compared to non-atrophic areas, and as can be evidenced by FIG. 41, plot B, the change in MEP values can not be represented by distance alone. This effect is non-linear and position dependent; and as such can not be captured by a single function as the current modifications will be patient specific and dependent upon the coil position relative to the specific tissue distribution.

Even though a decay of the current density magnitude versus the scalp-to-coil distance was shown, a simple linear increase in the applied TMS intensity should not be pursued in the clinical setting, even if there is no perturbation in the current location, because oftentimes brain atrophy is also associated with a change in brain activity and therefore the remaining active neurons in the atrophic brain might have an increased excitability and thus respond to smaller amounts of electric current. Indeed it has been demonstrated before that patients with Alzheimer disease and Parkinson's disease have a hyperexcitability of the motor cortex. Although the mechanism of this increased excitability remains uncertain, this phenomenon of an increased excitability is another parameter important in rTMS studies in these patients.

In addition to the magnitude changes, the vector current density orientation in the cortex was altered proximal to the widened sulci. With such changes, different neural elements could also be activated proximal to the sulci. As examined in the stroke study, there are numerous theories concerning the direction of the current vectors and which cortical neurons are stimulated, but it is clear that the directionality of the induced currents plays a clear role in which neurons are stimulated.

This study also provides evidence that the focus of induced current from TMS can be appreciably diminished in the region of widened sulci. In the healthy head and the symmetric atrophy models, the maximum cortical current density was always confined to a single discrete cortical location and the distribution was generally predicted based on the figure-of-eight coil configuration. However in the widened sulci models, there were many solutions with multiple disjoint areas, around the sulcal borders, where the current density was near its maximum or greatly increased as compared to the corresponding atrophy models without the widened sulci. In cases where the coil is placed more proximal or overlying the widened sulci, the current density distributions will be even less predictable than those accounted for with the coil positions that were implemented (dependent upon the individual geometry and electrical makeup of the sulci) as has been seen in the case of stroke. Additionally, such effects will in theory be found along normal sulcal regions to varying degrees dependent on the geometry and electrical tissue distributions in the regions of the sulci. In such cases of perturbation, stereotactic tracking systems which do not account for the field-tissue interactions will misrepresent the expected location and degree of stimulation in multiple cortical areas.

In the case where displacement currents are included (i.e., the relative permittivity scheme was adjusted to 10⁷εₒ), the overall trends are still the same. The magnitude of the induced current density changes proportionally with the magnitude of the complex tissue impedance. However, the same trends and effects discussed without the inclusion of displacement currents still appear with their inclusion. Thus, the same conclusions that the induced currents in the cortex can be modified in magnitude, location, and orientation in situations of brain atrophy are unchanged (and as such the inclusion of displacement currents does not substantially change the predicted site or degree of stimulation because the process is highly conductive), their inclusion ultimately will need to be discussed on how displacement currents influence cellular dynamics.

This foregoing demonstrates that TMS induced currents in the cortex can be modified in magnitude, location, and orientation in situations of brain atrophy. These cortical current density perturbations could prove to be dangerous or at the very least lead to unreliable results if guided by conventional methods based on healthy head models or with simplified models of atrophy which ignore electromagnetic field-tissue interactions. While the effects of cortical pathologies on the induced current densities were examined, many of the effects on the current densities are seen in the case of sub-cortical pathologies. As with the cortical pathologies, the current densities induced in the cortex in the presence of sub-cortical pathologies will need to be evaluated in a case by case basis.

Misconceptions and inconsistencies permeate the TMS literature, but the state of tDCS stimulation is even less defined. There is actually no consensus as to whether currents of sufficient magnitude actually reach the cortex in tDCS. This proposed component of the thesis will attempt to develop a fundamental understanding of the physical basis for tDCS by developing one of the only electromagnetic models of tDCS in a realistic healthy human head model and attempt to explain the underlying electrophysiology. During this modeling experiment, stroke pathology models will also be examined and compared to the healthy head models. And finally, the last experiment proposed for this thesis will mirror the TMS clinical treatment of stroke in stroke patients, but with TMS stimulation replaced with tDCS stimulation.

The investigation of the utility of Transcranial Direct Current Stimulation (tDCS) in clinical practice has been growing, however the knowledge about its effects and mechanisms of action remains limited. This paper presents a realistic magnetic resonance imaging (MRI)-derived finite element model of currents applied to the human brain during tDCS. Current density distributions were analyzed in a healthy human head model with varied electrode montages, which differed in electrode placement and size. For each solution, we analyzed the magnitude and the location of the maximum cortical current density, the maximum cortical current surface area, the current density vector behavior, and the changes in current density throughout the tissues. Analogous studies were completed for three pathological models of cortical infarcts. The current density magnitudes injected in the cortex by 1 mA tDCS ranged from 0.077 to 0.20 A/m². The pathological models revealed that cortical strokes, relative to the non-pathological solutions, can elevate current density maximums and alter their location. These results provide novel information that may guide optimized tDCS for application in normal subjects and patients with focal brain lesions.

Transcranial Direct Current Stimulation (tDCs) is a non-invasive brain stimulation technique that utilizes low amplitude direct currents applied via scalp electrodes to inject currents in the brain and thus modulates the level of excitability. However, little has been done to quantify the current densities injected during stimulation, to compare them to published current density magnitudes necessary for neural stimulation, or to analyze how different stimulation parameters can influence the stimulating currents.

Therefore we analyzed several magnetic resonance imaging (MRI)-derived finite element models (FEM) of electrical current applied to the human cortex during tDCS to: (i) Determine cortical current density distributions (magnitude and orientation) from various electrode configurations and source intensities that were based on electrode montages used in clinical investigations; (ii) Determine the role that human head tissue heterogeneities and anatomical variations play on the final current density distributions and the regions of the brain that are stimulated; (iii) Ascertain the effects of anatomo-pathological alterations that occur in stroke on the stimulating cortical current densities.

Multiple MRI derived finite element head models using different electrodes montages were constructed and the current densities were evaluated. The healthy head model, detailed below, was generated from an MRI of a thirty-eight year-old male with no neurological abnormalities. The MRI was obtained on a Siemens Magneton Vision 1.5 T scanner and the file saved in Analyze format (256×256×160, 1 mm³ voxel size). The stroke models were generated by altering the healthy head model's cortical geometry guided by the MRIs of stroke patients, the details of which will be discussed below.

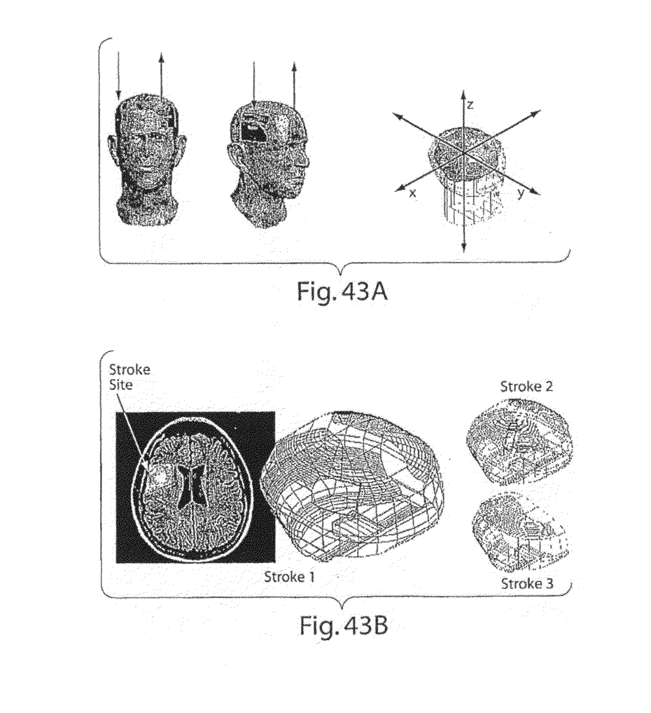

As discussed above, an initial sinusoidal steady state FEM was developed using the Ansoft 3D Field Simulator software package with the conduction solver. The FEM geometrical mesh structure was constructed from an MRI guided three-dimensional CAD rendering of the human head to form the healthy head model. FIG. 43, plot A is an example of the healthy head model with the anode at the right primary motor cortex (M1)-cathode on the left supraorbital electrode montage (Montage 1A). In the right most part of the figure the coordinate system is defined in the image that shows the outline of FEM mesh of the skin and the gray matter surface current density solution for this solution. FIG. 43, plot B illustrates gray matter FEM mesh outline for Stroke 1 and a slice from the MRI used to develop the model are shown in the left part of the figure. The gray matter mesh outlines for Stroke 2 and 3 are shown on the right. This model was generated to include the skin, skull, cerebral spinal fluid (CSF), gray matter, and white matter. The tissue conductivities of the base healthy head model were assigned the mean value from multiple references (skin: 0.465 S/m; bone: 0.010 S/m; CSF: 1.654 S/m; gray matter: 0.276 S/m; white matter: 0.126 S/m; see Table 1).

The Ansoft FEM solver was set to solve for the current densities in terms of the electric potential (φ), by solving the equation: ∇·(σᵢ∇φ)=0, where σᵢ is the conductivity of the tissue. The solution method followed an adaptive iterative process with convergence limits determined by the energy error in the system. The following specific electrode montages and head models were conducted to explore the effects of electrodes, tissues, and pathologies on tDCs stimulating currents.

The effects of varying the area of the surface electrodes on the tDCs current densities were analyzed. Rectangular electrode pairs of 7×7 cm², 7×5 cm², 5×5 cm², and 1×1 cm² were placed on the scalp overlaying the right M1 (anode) and on the forehead over the contralateral orbit (cathode). We chose these electrode sizes as several tDCS studies have been conducted in humans using 5×7 electrodes and 5×5 electrodes. We then chose extreme boundaries (7×7 and 1×1 electrodes) to explore further the effects of contact area.

The electrodes were modeled as planar current boundaries on the scalp surface, where 1 mA total current was applied at the anode location. For the 1×1 cm² electrode condition, currents of 0.0286 mA and 0.286 mA were also tested. The electrode placement schemes and average electrode current density magnitudes are included in Table 11 below, which includes various electrode montages. The Montage name is provided in the left column, anode and cathode distinction in the second column, electrode area in square centimeters in the third column, electrode placement in the fourth column, the total current in the closed loop circuit which drives stimulation (in mA), and the average current density at each contact point in the sixth column (in mA/cm²).

TABLE 11

| | | | | Total | Current |
|---|---|---|---|---|---|
| Scheme | electrode | size | placement | Current | density |
| Montage 1A | anode | 7 × 7 | Right M1 | 1 | 0.020 |
| | cathode | 7 × 7 | Left Orbital | | −0.020 |
| Montage 2A | anode | 5 × 7 | Right M1 | 1 | 0.029 |
| | cathode | 5 × 7 | Left Orbital | | −0.029 |
| Montage 3A | anode | 5 × 5 | Right M1 | 1 | 0.040 |
| | cathode | 5 × 5 | Left Orbital | | −0.040 |
| Montage 4A | anode | 1 × 1 | Right M1 | 1 | 1.000 |
| | cathode | 1 × 1 | Left Orbital | | −1.000 |
| Montage 5A | anode | 1 × 1 | Right M1 | 0.286 | 0.286 |
| | cathode | 1 × 1 | Left Orbital | | −0.286 |
| Montage 6A | anode | 1 × 1 | Right M1 | .029 | 0.029 |
| | cathode | 1 × 1 | Left Orbital | | −0.029 |
| Montage 1B | cathode | 7 × 7 | Right M1 | 1 | −0.020 |
| | anode | 7 × 7 | Left Orbital | | 0.020 |
| Montage 2B | cathode | 5 × 7 | Right M1 | 1 | −0.029 |
| | anode | 5 × 7 | Left Orbital | | 0.029 |
| Montage 3B | cathode | 5 × 5 | Right M1 | 1 | −0.040 |
| | anode | 5 × 5 | Left Orbital | | 0.040 |
| Montage 4B | cathode | 1 × 1 | Right M1 | 1 | −1.000 |
| | anode | 1 × 1 | Left Orbital | | 1.000 |
| Montage 5B | cathode | 1 × 1 | Right M1 | 0.286 | −0.286 |
| | anode | 1 × 1 | Left Orbital | | 0.286 |
| Montage 6B | cathode | 1 × 1 | Right M1 | .029 | −0.029 |
| | anode | 1 × 1 | Left Orbital | | 0.029 |
| Montage 7 | anode | 5 × 7 | Right M1 | 1 | 0.029 |
| | cathode | 5 × 7 | Left M1 | | −0.029 |
| Montage 8 | anode | 5 × 7 | Right DLPFC | 1 | 0.029 |
| | cathode | 5 × 7 | Left Orbital | | −0.029 |
| Montage 9 | anode | 5 × 7 | V1 | 1 | 0.029 |
| | cathode | 5 × 7 | Left Orbital | | −0.029 |
| Montage 10 | anode | 5 × 7 | Right M1 | 1 | 0.029 |
| | cathode | 5 × 7 | Left lower neck | | −0.029 |
| Montage 11 | anode | 5 × 7 | Right DLPFC | 1 | 0.029 |
| | cathode | 5 × 7 | Left DLPFC | | −0.029 |
| Montage 12 | anode | 5 × 7 | V1 | 1 | 0.029 |
| | cathode | 5 × 7 | Vertex | | −0.029 |
| Strokes 1-3 | anode | 5 × 7 | Right M1 | 1 | 0.029 |
| | cathode | 5 × 7 | Left Orbital | | −0.029 |
| Stroke 1B | anode | 5 × 7 | Right M1 | 1 | 0.029 |
| | cathode | 5 × 7 | Left M1 | | −0.029 |

Average electrode current density magnitudes are reported as the total current in the electrode divided by the total electrode area). The magnitude and location of the maximum cortical current density were evaluated for each electrode montage and individually for each electrode (i.e., the maximum cortical current density proximal to the anode was not always equivalent to the maximum near the cathode). Additionally, the surface area on the cortex where the current density was greater than 90% of its maximum value was calculated (i.e., if the maximum magnitude of the cortical current density was 1 A/m² for a given electrode scheme, then the area was calculated where the current ranged from 0.9-1.0 A/m²). We refer to this as the maximum cortical current surface area and report the area proximal to each electrode (i.e., there was a different area for the cathode and the anode).

Furthermore, the current density vector behavior was also analyzed in the tissues. Finally, the variation in the current density across the different tissues was evaluated. To quantify the shunting effect, we divided the average maximum skin current density by the maximum cortical current density, where the average maximum skin current density were defined as the current density magnitude on the skin for which at least 0.5 cm² in area was covered and which was not confined to the electrode boundary edge effects. For these electrode schemes, the polarity of the current was also reversed such that the anode became the cathode and vice-versa. The exact current density analysis was completed for the reversed polarity schemes.

The effects of varying the position of the surface electrodes on the tDCs current densities were analyzed. In these cases we modeled electrodes of an area of 5×7 cm² and an applied current of 1 mA. The following electrode placements were analyzed: (1) anode over the right M1-cathode over the left supraorbital region; (2) anode over the right M1-cathode over the left M1; (3) anode over the primary visual cortex (V1)-cathode over the vertex; (4) anode over V1-cathode over the left supraorbital region; (5) anode over the left dorso-lateral prefrontal cortex (DLPCF)-cathode over the right supraorbital region; and (6) anode over the right DLPFC-cathode over the left DLPFC. Additionally, solutions were obtained for a 5×7 cm² anode above the right M1 and a 2×2 cm cathode electrode in the contralateral lower neck. For each solution, the location and magnitude of the maximum cortical current density, the maximum cortical current surface area, the current density vector orientations, and the behavior of the current in the tissues were analyzed and compared (see FIG. 1A for an example of the anode over the right M1-cathode over the left supraorbital region electrode montage; the full list of montages is tabulated in Table 2).

Three stroke models of various geometries were implemented to compare the effects of different anatomy perturbations on the current injected by tDCS. To represent the infarction site in the FEM geometry, CSF was used to replace the damaged tissue as is shown by both imaging and histopathology studies in the post acute sta. As there have been no studies reporting the conductivity alterations at the gray matter white-matter interface proximal to the infarction region, the tissue was considered continuous cerebral tissue as shown in the conductivity values in Table 12 below (conductivity=0.276 S/m).

TABLE 12

| | Mean |
|---|---|
| Tissue | Conductivity (S/m) |
| Skin-Scalp | 0.465 |
| Bone-skull | 0.010 |
| Cerebral Spinal Fluid | 1.654 |
| Gray Matter | 0.276 |
| White Matter | 0.126 |

Strokes 1 and 2 were located in the right frontal lobe and modeled to represent infarctions of the superior branches of the right middle cerebral artery. Stroke 1 had an approximate volume of 18.5 cm³ with a maximum 2.5 cm inferior to superior length along the cortical face, a maximum anterior to posterior length of 3.5 cm along the cortical face, and a maximum depth of 2.8 cm measured from the cortical face. Stroke 2 had an approximate volume of 5.3 cm³ with a maximum inferior to superior length of 4 mm along the cortical face, a maximum anterior to posterior length of 4 mm along the cortical face, and a maximum depth of 4 mm measured from the cortical face.

Finally, Stroke 3 represented a large stroke due to left MCA occlusion with poor collateral perfusion due to atherosclerotic vascular disease; its size was approximately 350 cm³ and it was designed by removing the cortical mantle of the left hemisphere from the model. The stroke models differed from the healthy head model only at the infarction location, where the electrical properties were set to correspond to those of CSF as opposed to gray matter (see FIG. 43, plot B).

In these stroke models, solutions were obtained for electrode pairs (area 5×7 cm², 1 mA) placed over the right M1 (anode)-contralateral orbital region (cathode). We also studied Stroke 1 with the anode over the right M1-cathode over the left M1 (5×7 cm², 1 mA) electrode montage (we refer to this as Stroke 1B). For all of the cases, we analyzed the magnitude of the maximum cortical current density, the location of the maximum cortical current density, the maximum cortical current surface area, and the injected current density vector behavior. The solutions were then compared to the analogous healthy head model.

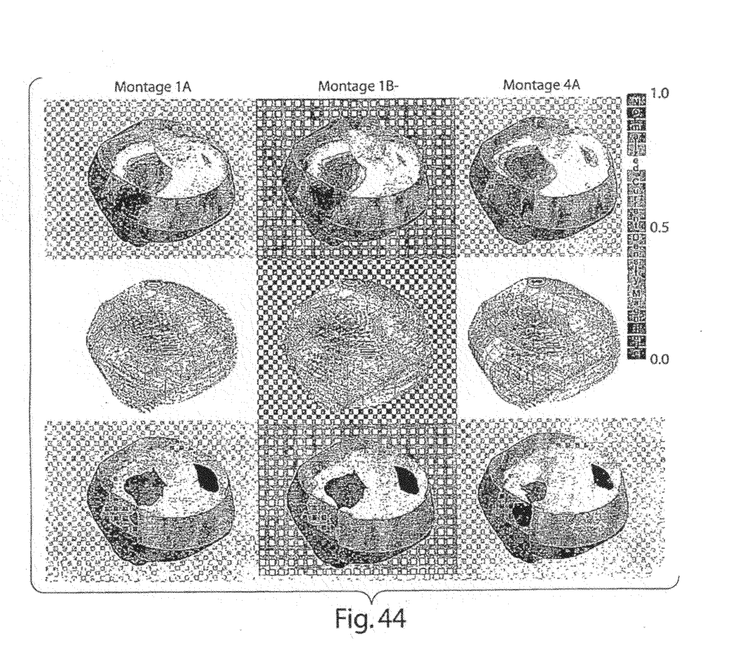

FIG. 44 illustrates cortical current densities for montages 1A, 1B, and 4A in the healthy head model. The top row displays the cortical current density along the surface of the gray matter for each solution. The second row displays the current vector distributions on the cortical surface. Note that the scale for the top two rows is normalized to the maximum cortical current density for each separate solution. The third row displays the maximum cortical current surface areas for the anode and the cathode.

For the varied size electrode montages in the healthy head model with the anode placed above M1 and the cathode above the contralateral orbital region, the maximum cortical current densities ranged from 0.2-0.0032 A/m². Using an injection current of 1 mA, the greatest maximum cortical current density (0.2 A/m²) was found for the 5×7 cm² electrode scheme with the anode over the vertex and the cathode over V1. The lowest maximum cortical current density was found for the 1×1 cm² electrode scheme with the 0.0286 mA total injected current (this current density was equivalent to the average electrode current density of the 5×7 cm² electrode schemes, but this setup implemented the lowest total injected current).

For the 1×1 cm² electrode schemes with decreasing current strengths, the maximum cortical current density magnitudes decreased linearly with the overall total injected current. The locations of cortical maxima were always within the region of the electrode, essentially lying along the superior portion of the motor strip (FIG. 44). For the cases where the anode and cathode were reversed, the magnitudes remained the same for the electrode location, but with the polarity reversed. These results are tabulated in Table 13, which tabulates current density magnitudes: The first column reports the electrode Montage, the second problem the specific electrode, the third reports the Maximum Cortical Current Density (MCCD) in A/m², the fourth and fifth report the location of the MCCD, the sixth reports the area of the maximum cortical current.

| Montage | electrode | MCCD | location | | area | AMSCD | Shunting |
|---|---|---|---|---|---|---|---|
| Montage 1A | anode | 0.091 | (36.2, 17.75, 32.89) | motor strip | 13.7 | 0.79 | 8.68 |
| | cathode | 0.081 | (−16.7, 54.7, 32.1) | frontal lobe | 3.31 | 0.936 | 11.56 |
| Montage 2A | anode | 0.098 | (47.1, 27.5, 26.9) | motor strip | 11.25 | 0.98 | 10.00 |
| | cathode | 0.084 | (−14.5, 50.8, 27.3) | frontal lobe | 3.06 | 1.22 | 14.52 |
| Montage 3A | anode | 0.103 | (38.2, 26.0, 29.7) | motor strip | 7.78 | 1.12 | 10.87 |
| | cathode | 0.088 | (−16, 55, 31.3) | frontal lobe | 2.85 | 1.28 | 14.54 |
| Montage 4A | anode | 0.144 | (53.5, 22.75, 11.3) | motor strip | 3.3 | 5.2 | 36.11 |
| | cathode | 0.112 | (14.4, 51.48, 31.6) | frontal lobe | 1.65 | 9.7 | 86.61 |
| Montage 5A | anode | 0.043 | (54.8, 20.3, 11.6) | motor(inferior) | 3.25 | 1.38 | 32.09 |
| | cathode | 0.033 | (−17.2, 56.2, 32.0) | frontal lobe | 1.63 | 3.33 | 100.91 |
| Montage 6A | anode | 0.0043 | (54.9, 19.9, 11.2) | motor(inferior) | 3.24 | 0.133 | 30.93 |
| | cathode | 0.0032 | (−17.2, 56.2, 32.0) | frontal lobe | 1.52 | 0.342 | 106.88 |
| Montage 1B | cathode | 0.091 | (36.2, 17.75, 32.89) | motor strip | 13.7 | 0.79 | 8.68 |
| | anode | 0.081 | (−16.7, 54.7, 32.1) | frontal lobe | 3.31 | 0.936 | 11.56 |
| Montage 2B | cathode | 0.098 | (47.1, 27.5, 26.9) | motor strip | 11.25 | 0.98 | 10 |
| | anode | 0.084 | (−14.5, 50.8, 27.3) | frontal lobe | 3.06 | 1.22 | 14.52 |
| Montage 3B | cathode | 0.103 | (38.2, 26.0, 29.7) | motor strip | 7.78 | 1.12 | 10.87 |
| | anode | 0.088 | (−16, 55, 31.3) | frontal lobe | 2.85 | 1.28 | 14.55 |
| Montage 4B | cathode | 0.144 | (53.5, 22.75, 11.3) | motor strip | 3.3 | 5.2 | 36.11 |
| | anode | 0.112 | (−14.4, 51.48, 31.6) | frontal lobe | 1.65 | 9.7 | 86.61 |
| Montage 5B | cathode | 0.043 | (54.8, 20.3, 11.6) | motor(inferior) | 3.25 | 1.38 | 32.09 |
| | anode | 0.033 | (−17.2, 56.2, 32.0) | frontal lobe | 1.63 | 3.33 | 100.91 |
| Montage 6B | cathode | 0.0043 | (54.9, 19.9, 11.2) | motor(inferior) | 3.24 | 0.133 | 30.93 |
| | anode | 0.0032 | (−17.2, 56.2, 32.0) | frontal lobe | 1.52 | 0.342 | 106.88 |
| Montage 7 | anode | 0.104 | (38.6, 0.5, 31.1) | motor(superior) | 16.8 | 0.92 | 8.85 |
| | cathode | 0.104 | (−38.5, 0.4, 31.1) | motor(superior) | 16.8 | 0.92 | 8.85 |
| Montage 8 | anode | 0.093 | (26.3, 51.9, 28.9) | frontal lobe | 6.4 | 1.07 | 11.51 |
| | cathode | 0.086 | (−17.1, 56.6, 32.2) | frontal lobe | 5.84 | 1.17 | 13.60 |
| Montage 9 | anode | 0.096 | (−1.52, −63.2, −13.3) | V1 | 18.7 | 0.87 | 9.0625 |
| | cathode | 0.079 | (−30.8, 42.9, 32) | frontal lobe | 16.2 | 1.11 | 14.05 |
| Montage 10 | anode | 0.0877 | (55.8, 16.6, −8.9) | motor(inferior) | 4.78 | 0.92 | 10.49 |
| | cathode | | | None | | 2.87 | |
| Montage 11 | anode | 0.077 | (18.0, 62.7, 27.97) | frontal lobe | merged | 1.33 | 17.27 |
| | cathode | 0.077 | (−18.0, 62.7, 28.0) | frontal lobe | merged | 1.33 | 17.27 |
| Montage 12 | anode | 0.2 | (0, −20.1, 37.8) | merged above v1 | 3.25 | 1.17 | 5.85 |
| | cathode | 0.2 | | | | 1.43 | 7.15 |

The maximum cortical current surface areas, defined as the cortical surface area where the current ranged from 90% to 100% of its maximum, ranged from 3.24 to 13.7 cm² for the electrode over the M1 and ranged from 1.48 to 3.36 cm² for the electrode over the contralateral orbital (FIG. 44). The areas were always greater when the electrode was placed over M1 than over the contralateral orbital region. In general, when the electrode was placed over M1, the maximum cortical current surface areas increased with increasing electrode surface area. However, when the electrode was placed above the contralateral orbital region, the maximum cortical current surface areas did not vary much between the 7×7 cm², 5×7 cm², and 5×5 cm² electrode schemes (slightly decreasing with decreasing surface area), and only decreased by a factor of approximately two for the 1×1 cm² electrodes. For the 1×1 cm² electrode montages with decreasing current strengths, the maximum cortical current surface areas were essentially unchanged with decreasing current strengths, but note that the areas are reported relative to the maximum current density magnitude on the cortex. These results are tabulated in Table 13.

The current density vector distribution followed the same course and orientation for essentially all anode M-1 cathode contralateral orbital region schemes with varied electrode sizes (with differences in the relative magnitudes). The greatest difference was observed between the 7×7 and 1×1 cm² electrode schemes, where the larger electrode surface area corresponded to a less focal distribution. For all electrode schemes, the largest currents were oriented along the superior part of the right motor strip across the hemispheres and through the left superior medial frontal lobe (see FIG. 44 for a graphical representation of the distributions). When the polarity of the sources was reversed (i.e., the anode and the cathode were reversed), the current density orientations were systematically reversed in polarity with the orientations 180 degrees out of phase with the non-reversed polarity solutions (See FIG. 44).

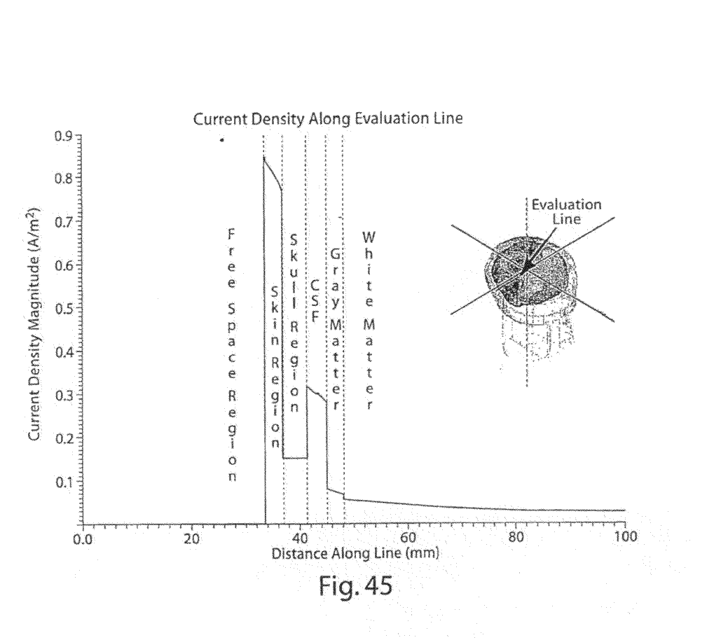

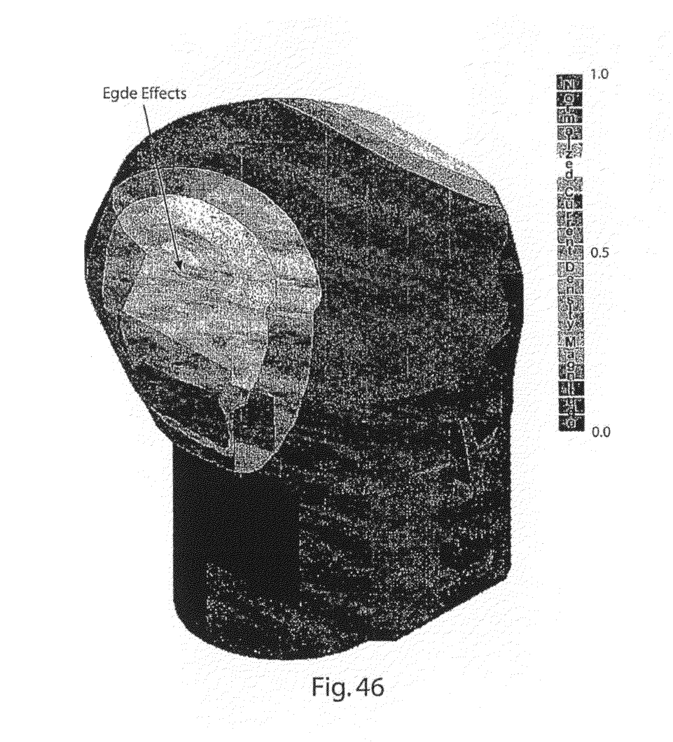

FIG. 45 illustrates current density behavior through tissues, and more particularly, current density magnitude evaluated along an evaluation line in the healthy head model for Montage 1A. The inset shows mesh model with the current density magnitude plotted on the surface of the cortex with the evaluation line shown intersecting the tissues—the current density magnitudes displayed in the primary graph were calculated along this line. Note that the current density magnitude varies with the conductivity of the tissues. FIG. 46 illustrates skin surface current density for electrode montage 7. Note the drastic edge effect at the electrode boundary location, current density of 2.06 A/m², compared to the average maximum skin current density of 0.92 A/m².

The current density magnitudes varied substantially throughout the tissues, and stair step jumps in the current density occurred at each tissue boundary (see FIG. 45). The largest current density magnitude was located near the edge of the electrodes on the skin surface. These largest current density values on the skin were restricted to small areas, were not reflective of the average current densities magnitudes on the skin or of the shunting effects along the skin (see FIG. 46), and have been explored in depth by other researchers. The average maximum skin current densities increased with decreasing electrode surface area. To quantify the shunting effect, we divided the average maximum skin current density by the maximum cortical current density. There were drastically greater levels of shunting for the 1×1 cm² electrodes than for the larger electrode schemes. These shunting levels ranged from 30.2-106.9 for the 1×1 cm² montages to 8.7-14.5 for the larger electrodes. These values are presented in Table 13.

We maintained the electrode size fixed (using 5×7 cm² electrodes as this is the size of electrodes most commonly used in published clinical studies) and varied the position of the electrodes in the healthy head model. The maximum cortical current densities ranged from 0.077-0.20 A/m² at the anode and at the cathode. The locations of cortical maxima were within the tissue underlying the electrodes. These results are tabulated in Table 14, which tabulates current density magnitudes for the stroke models: The first column reports the electrode scheme, the second problem the specific electrode, the third reports the Maximum Cortical Current Density (MCCD) in A/m², the fourth and fifth report the location of the MCCD, the sixth reports the area of the maximum cortical current density, the seventh reports the Average Maximum Skin Current Density, and the final reports the extent of shunting for each electrode scheme.

TABLE 14

| Montage | electrode | MCCD | location | | area | AMSCD | Shunting |
|---|---|---|---|---|---|---|---|
| Stroke 1 | anode | 0.127 | (56, 18.2, 17.5) | stroke | 1.1 | 0.97 | 7.64 |
| | | | | border | | | |
| | cathode | 0.081 | (−29.8, 77.6, 14.5) | frontal | 5.12 | 1.3 | 16.05 |
| | | | | lobe | | | |
| Stroke 2 | anode | 0.116 | (53.4, 25.8, 27.3) | stroke | 1.6 | 1 | 8.62 |
| | | | | border | | | |
| | cathode | 0.08 | (−29.63, 68.9, 11.7) | frontal | 4.3 | 1.26 | 15.75 |
| | | | | lobe | | | |
| Stroke 3 | anode | 0.11 | (56, 27.83, 1.81) | Motor | 3.17 | 1.06 | 9.64 |
| | | | | (lower) | | | |
| | cathode | Cortex removed | | | | 1.23 | |
| | | under cathode | | | | | |
| Stroke 1B | anode | 0.131 | (56.2, 18.0, 16.3) | stroke | 1.2 | 0.97 | 7.40 |
| | | | | border | | | |
| | cathode | 0.106 | | | 16.4 | 0.93 | 8.77 |

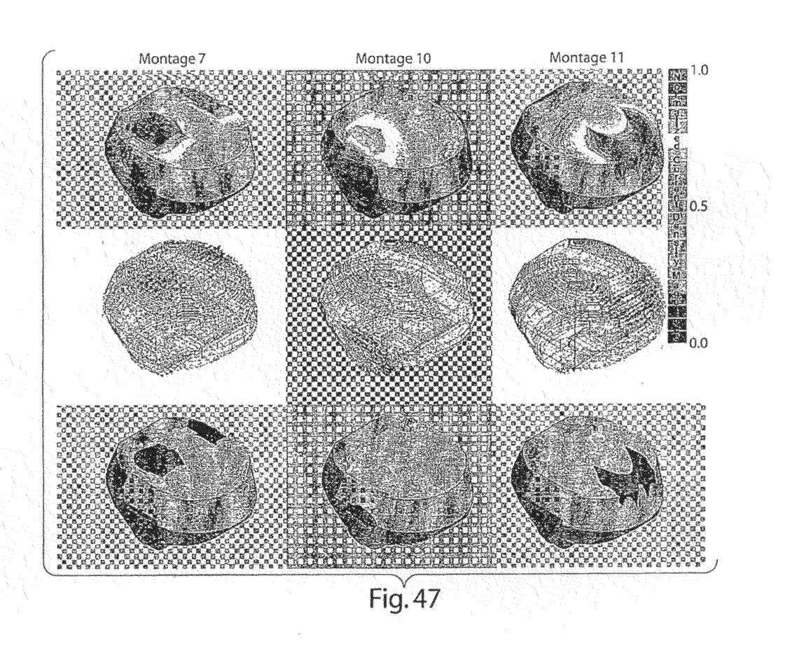

FIG. 47 illustrates cortical current densities for montages 7, 10, and 11 in the healthy head model. The top row displays the cortical current density along the surface of the gray matter for each solution. The second row displays the current vector distributions on the cortical surface. Note that the scale for the top two rows is normalized to the maximum cortical current density for each separate solution. The third row displays the maximum cortical current surface areas for the anode and the cathode.

The maximum cortical current surface areas ranged from 3.25 to 18.7 cm² at the anode and from 3.06 to 16.8 cm² at the cathode. For the ‘anode over the right DLPFC-cathode over the left DLPFC’ and for the ‘anode over V1-cathode over vertex’ electrode montages, the areas of maximal cortical current were merged at the electrode sites in the sense that there was no clear demarcation of the maximum cortical current surface density under the anode and the cathode (see FIG. 47). The current density vector distributions varied considerably between the various electrode schemes and are graphically displayed in FIG. 47. As in the other solutions, the current density magnitudes varied substantially throughout the tissues, with the largest current density magnitude always observed near the edge of the electrodes on the skin surface.

The current density distributions were altered in the three stroke models compared to the healthy head model with the same electrode scheme (‘anode over right M1-cathode over orbital region’, area 5×7 cm², 1 mA). The current density maximum was slightly larger in each of the solutions (see Table 5) for the stroke than the healthy head models. The current density maxima were located at different locations in these solutions compared to the healthy head model; at the boundary of the infarction in the Stroke 1 and Stroke 2 models, and more lateral and inferior in the Stroke 3 model. For the Stroke 1 and Stroke 2 models, where the current density maxima were found at the infarction boundary, the maximum cortical current density areas were more focal than in the non-stroke case.

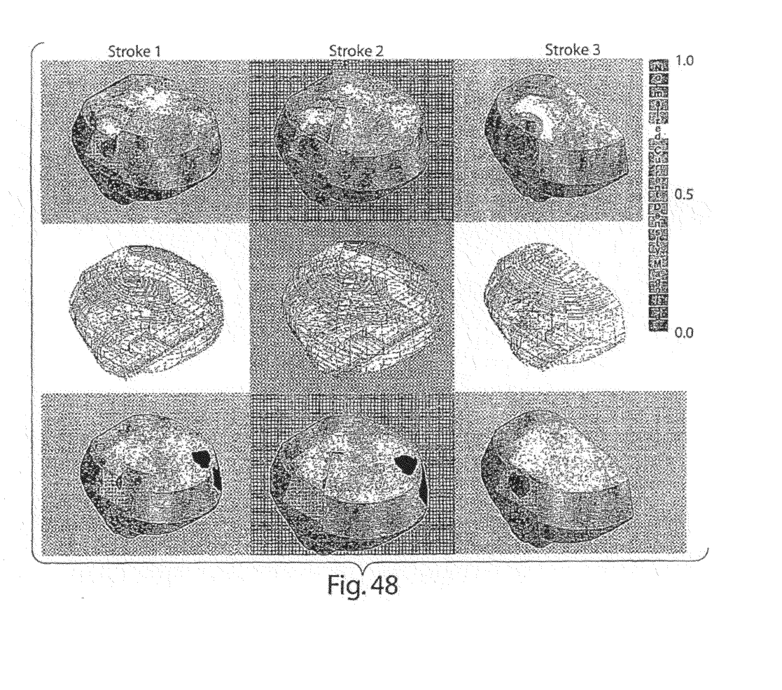

FIG. 48 illustrates cortical current densities for strokes 1-3. The top row displays the cortical current density along the surface of the gray matter for each solution. The second row displays the current vector distributions on the cortical surface. Note that the scale for the top two rows is normalized to the maximum cortical current density for each separate solution. The third row displays the maximum cortical current surface areas for the anode and the cathode. Note the concentration of current density along the infarction border in Strokes 1 and 2.

For Stroke 1B, the current density behavior in the region of the stroke was similar to that of Stroke 1, and was similar in behavior around the cathode to that Montage 7. For all of the solutions, the current density vector orientations were clearly altered at the infarction border (see FIG. 48). Additionally, the current density distributions were nearly unchanged on the surface of the skin, but substantially different in both the CSF and cerebral tissue in the infarctions as compared with the normal condition (see Table 14). Therefore, in applying tDCS to patients with stroke, just as with other forms of brain stimulation, it seems critical to consider the location, geometry, and tissue characteristics of the lesion, as adjustments of the stimulation montage might be needed to affect the desired cortical target.

The foregoing explored the behavior of the currents injected in human brain models by tDCs. Our models were based on a finite element electromagnetic solver integrated with MRI derived head models (including three different stroke cases). The model focused on injected cortical current densities, and explored the effects of varied electrode montages and varied electrode sizes, the shunting effect in the tissues, and the effects of stroke on the stimulating current density distributions.

In the classic sense of the term, stimulation implies the active initiation of an action potential via an outside stimulus. In controlled electrical stimulation of the cortical neurons, thresholds reported for stimulation of cortical cells range from approximately 22 to 275 A/m², values far exceeding those we report in this model. Given such drastic differences it is not surprising that some researchers have questioned the biological effects of tDCS, especially when compared to current magnitudes needed for direct current stimulation of the exposed cortex.

However, it is possible that tDCs does not actively stimulate the cortex in the classic sense of the term, but rather it ‘modulates’ or modifies the cortical excitability. In the early 1960's, DC current amplitudes as low as 0.25 A/m² applied to the exposed pia via surface electrodes (3 μA from 12 mm² saline cup on exposed pia surface) were shown to influence spontaneous activity and the magnitude and characteristics of evoked response of neurons for hours after just minutes of stimulation in rat preparations. Note that in these studies the stimulus was applied through the pia and as such, given the expected current spread, the current density magnitudes at the cortical neurons would be expected to be lower and possibly similar to what has been shown by our models. Thus, our models confirm that tDCS infects an electric current into the brain that has the necessary magnitude to cause biological effects even though it is unlikely to directly depolarize neuronal elements and induce action potentials. This finding is important to guide the interpretation of the behavioral effects induced by tDCS that have been demonstrated by previous investigations.

The change in the magnitude of the cortical current density maxima depended on the location and the area of the stimulating electrodes. In the case where the current strength was altered and the electrodes remained the same in size, the cortical current density maxima decreased linearly with a decrease in the overall injected current. In the case where the electrode sizes were varied with similar scalp locations, the current density maxima decreased with increasing electrode size. Additionally, as the area of the electrodes decreased, the degree of shunting along the skin increased, indicative of an altered resistive matrix of the head system.

The location of the maxima was also dependent on the electrode placement, which is indicative of the paths of current flow relative to the resistive matrix of the head. For example, in the ‘anode over the right M1-cathode over the left orbital region’ electrode scheme, the magnitude of the cortical current density was greater in M1, regardless of the electrode polarity. In the situations where the anode and cathode were placed at homologues locations over both hemispheres (e.g. over both motor cortices) there is clearly no difference in the magnitudes of cortical currents under the anode and cathode; a reversal in the polarity of the electrodes changed the polarity of the induced current by 180 degrees without affecting the magnitude of the cortical current density.

In general the maximum cortical current density was greater when the electrodes were placed along flatter cortical surfaces with less curvature of the head system, and when the curvature was maximized, less current penetrated into the cortex and thus the shunting effect increased. This is to say that the resistive networks of the head in our models were such that the current paths flowed more through the skin and the outer layers proximal to electrode locations along more curvilinear scalp locations. Based of this finding, one has to judge carefully the reliability of spherical models, which have been used as the basis for tDCs modeling in the past. Symmetry conditions can lead to anomalous current density solutions while modeling stimulation situations which do not reflect the anatomy under consideration.

We also show that the cortical currents injected by 5×7 cm² versus 5×5 cm² electrodes are very similar (less than 5% difference), therefore the decision between the two types of electrodes might not have a significant clinical impact. In fact, the two clinical tDCS studies on stroke used different electrodes size (5×5 cm² and 5×7 cm²) and showed similar motor function improvement. And, as we have shown that shunting effects increase with decreasing electrode area (see Montagel A vs Montage 4A), it is possible that some of the early discrepancies seen in the literature were simply related to unreported differences in electrode design. For example, compare the work of Lippold et al. to that of Theano et al, and note that the inconsistent results appear to be obtained with the same experimental design, however neither reports their electrode sizes and as we have shown this could clearly have had an impact on their results.

The various electrode montages analyzed showed similar cortical current density magnitudes for all of the cases except for the 1×1 cm² electrode schemes with lower levels of total injected current. The maximum cortical current density was found for the ‘anode over V1-cathode over the vertex’ electrode montage. In this case the electrodes were over a location on the cortex that was fairly flat and the electrodes were very close (less than 3.5 cm at shortest distance).

However, it appears that the distance between the electrodes is less important than the overall relative location because in the ‘anode over the right DLPFC-cathode over the left DLPFC’ electrode montage where the electrodes were also very close (less than 3.8 cm at shortest distance), we found the lowest cortical current density for 1 mA overall injected current. In the case of the ‘anode over the right M1-cathode over the left M1’, the current density magnitudes in the cortex were actually larger than the case where the second electrode was placed over the contralateral orbital region. This is again indicative of the resulting current density paths and the overall resistive matrix of the head tissue network.

Since the 1960's it has been known that the polarity, thus the orientation, of the current densities is a key determinant in whether the cortex is facilitated or suppressed (i.e., whether the spontaneous activity is increased or decreased and whether the magnitudes of evoked potentials are greater or smaller following stimulation). Previous studies showed that surface positive cortical polariziation (anode placement) excites the cortex and the opposite effect is seen with surface negative polarization. Landau et al. showed that the surface effects could be reversed by placing the stimulating source within the cortex, such that the current density orientations are reversed relative to the stimulated neurons.

In addition, Terzuolo et al, showed a similar effect in neural preparations. They observed that the change in the neural firing frequency with weak DC currents could be modulated based on the relative current to neural orientations. In more recent tDCs studies on motor cortex stimulation, Nitsche and Paulus showed that stimulation with the cathode placed above the motor cortex suppresses subsequent cortico-spinal responses, whereas facilitation of corticospinal responses is observed with the anode placed above the motor cortex.

Thus, with the different electrode schemes studied, one can clearly see that changing the polarity of the electrodes is not the only way to alter the orientation of the injected current densities, however it may be the most efficient. Additionally, it is possible that multipolar electrode schemes could be devised which could more accurately focus the currents and future research based on this methodology should be explored.

Fields were altered in the pathological stroke condition, relative to the healthy head situation, such that the current density maximums were elevated and located directly along the stroke border for Stroke 1 and Stroke 2, and such that the current density maximum was elevated and found more inferior along the cortical surface for Stroke 3. The fields were constrained by a new set of boundary conditions in the region of the infarction. In our model we replaced the cortical cut with CSF, providing a different conductive path for the currents away from the maximum current density location in the healthy head model. The kind of perturbations observed in the stroke models might occur in other pathological cases in which the geometry or electrical characteristics of brain tissue are altered. Therefore, in applying tDCS to patients with stroke, just as with other forms of brain stimulation, it seems critical to consider the location, geometry, and tissue characteristics of the lesion, as adjustments of the stimulation montage might be needed to affect the desired cortical target.

By exploring the current densities injected into the cortex during tDCs, this paper provides a foundation based on physics by which one can guide future clinical studies and explore fundamental aspects of this technique. We have shown that the location of the cathode relative to the anode is important in determining the final current density distribution. In the healthy head model the magnitude of the cortical current density in the region of the cortex under a single electrode is primarily dependent on the scalp position of the electrode, the electrode size, the injected current density, and the relative electrical and anatomical properties of the tissues in the region. However, the current density orientations are dependent on the position of both electrodes.

For example, if one compares Montage 2A (right M1 (anode) and contralateral orbit (cathode)) with Montage 7 (right M1 (anode) and contralateral M1 (cathode), see FIGS. 44 and 47) one will note that magnitude of the cortical current densities are not drastically different between the two montages (0.098 vs. 0.104 A/m² at the anode and 0.084 0.104 A/m² at the cathode respectively) but that the orientation of the current density vectors point in different directions (Montage 2A sees cortical currents directed in a more dorsal to ventral orientation than the lateral to medial currents seen in Montage 7A, see FIGS. 44 and 47). This type of information provided by our modeling approach can guide future clinical studies.

For example, clinical investigators could use the current density magnitude information to establish that induction of relatively selective changes in neural excitability in one M1 can be best induced with Montage 1A, or that Montage 7 is most likely to induce comparable changes in both the right and left M1. Additionally a more pronounced effect in M1 can be achieved with Montage 7 than Montage 1A due to the increased cortical current density. From the general polarity of the currents, one would expect an overall inhibitory response under the cathode and a facilitatory response under the anode for both montages 1A and 7, but one could expect more subtle local changes based on the differences in the current density orientations. M1 is located under the anode in both montages, but the orientation of the currents in M1 depends on the cathode location and is thus different for each montage (see FIGS. 44 and 47).

As previously discussed, the relative current to axonal axis orientation is important in determining the degree of neural excitability changes, and thus with these two facts it is possible to expect that different neural populations would be affected unequally. With the broad electrodes currently used in tDCs it is difficult to say how easy it will be for a clinician to harness these effects, but with future improvements in the technique such focal control based on relative current to neural orientations might be possible. One could postulate the use of multiple electrodes of smaller dimensions and unequal current densities to influence neural populations based on their anatomical orientations relative to the calculated current densities in a more focal manner than is currently implemented.

Clearly, this technique does share the same focality as that seen with more invasive methods such as microstimulation, but tDCs will be superior to those techniques in cost, ease of use, and level of invasiveness. In the future, one could use the electromagnetics approach presented here to further increase the focality of tDCs without relying on complicated surgical procedures and costly studies. Additionally, one could use this approach to guide the clinician in stimulation when dealing with patients who suffer from pathologies that alter their cortical anatomy or conductive properties of the head tissues and as such one could conceive a clinical tracking system that predicts the current density distribution is patients relative to their individual MRIs.

Several studies have shown that another noninvasive technique for brain stimulation (repetitive transcranial magnetic stimulation or rTMS) might be useful in the treatment of depression. The leading hypothesis to account for the antidepressant effects of rTMS is that high-frequency rTMS leads to an increase in excitability in the targeted cortical region and thus, when applied to the left DLPFC in patients with depression it can normalize a pathological state of hypoactivity. Conversely, low-frequency rTMS over the homologous area in the right hemisphere is thought to induce a suppression of the targeted cortical region and thus decrease a relatively hyperactive right prefrontal cortex (as compared with the left side).

In both cases, the aim appears to be to normalize an interhemispheric imbalance in activity thought to be causally related to the mood disturbance. These notions are almost certainly too simplistic, however, the existing results would suggest that anodal tDCS of the left DLPFC or cathodal tDCS of the right DLPFC might both result in depression amelioration. Indeed tDCS treatment using the electrode montage of anodal-left DLPFC and cathodal-contralateral supraorbital (reference electrode) is associated with mood improvement in patients with treatment resistant depression.

The results of our study show that this electrode montage with the supra-orbital reference, will result in adequate current magnitude in the area under the active electrode (i.e. the DLPFC electrode). An alternative approach would be bilateral stimulation in which the excitability-enhancing anode electrode is placed over the left DLPFC and the excitability-diminishing cathode electrode is placed over the right DLPFC. Bilateral stimulation has been investigated before using rTMS, but due to technical limitations first one and then the other hemisphere were targeted.

However, with the technique of tDCS, bilateral stimulation can be performed simultaneously, placing anode and cathode over homologous regions of the right and left hemispheres. Our study shows that such a montage would lead to rather large shunting of currents given the proximity of both electrodes. Nevertheless, we show that the cortical current density in the DLPFC would be comparable for the “bilateral” montage when compared to the “unilateral” montage (anode on the DLPFC and cathode on the contralateral supraorbital).

In stroke, recent research suggests that facilitation of activity in the lesioned hemisphere and suppression of activity in the undamaged hemisphere might both have a desirable therapeutic impact and promote recovery of function in these patients [220]. Given these aims, three tDCS approaches would seem to be reasonable: (1) anodal tDCS of the affected hemisphere with the expectation that activity will be increased; (2) cathodal tDCS of the unaffected hemisphere with the aim of reducing cortical excitability; and (3) bilateral stimulation in which both motor cortices are stimulated simultaneously by applying anodal tDCS to the affected and cathodal tDCS to the unaffected hemisphere.

The results of our study show that the approaches (1) and (2) might induce significant and reliable currents in the cortex if the reference electrode is placed over the contralateral supraorbital area. This finding is in accordance with recent clinical data. Furthermore, our results show that bihemispheric stimulation will also induce currents in the motor cortices of adequate magnitude and thus may in fact, similar to the situation in depression, allow for an even greater desirable modulation of bihemispheric activity and larger behavioral effect size.

An important question when using tDCS in stroke is whether the stroke lesion would disturb the electric current similarly to what is observed in rTMS. However, when one compares tDCS with rTMS in the case of stroke it is apparent that there are large differences in the degree of perturbation of the stimulating currents caused by the infarction depending on the stimulation methodology. We show that, differently from rTMS, the cortical current densities injected by tDCS in head models of stroke remain relatively unchanged. There certainly is a disturbance to the injected currents, with an increased current density at the location of the infarction border, but these remain in the range of magnitudes of stimulation in the healthy head model. This is probably due to the fact that tDCs is inherently less focal than TMS. Note however, that while possibly an advantage in some instances, such relative non-focality might also prevent desirable effects. For example, recent work suggests that rTMS to the pars triangularis of the right frontal operculum in patients with non-fluent aphasia promotes language recovery, while the same rTMS to the neighboring right pars opercularis worsens speech and language. In such an instance, tDCS would likely fail to reveal any beneficial effects given the lack of focality of the currents revealed by our modeling work.

For the different electrode schemes studied, the calculated current density magnitudes are sufficient to conclude that in humans tDCs is indeed capable of altering ongoing cortical neural activity and evoked responses of the cortex during stimulation. Additionally, this study has demonstrated that tDCs effects are altered in the presence of cortical damage, though the effect is relatively small as compared with other brain stimulation techniques. The kind of perturbations observed in the stroke models will occur in other pathological cases in which the geometry or electrical characteristics of brain tissue are altered.

Recent investigation showed that anodal transcranial direct current stimulation (tDCS) of the affected hemisphere enhances motor function in stroke patients. Given that the ipsilateral undamaged hemisphere can sometimes play a critical role in stroke recovery, we hypothesized that suppression of the undamaged hemisphere using cathodal stimulation might promote recovery of motor function similar to anodal stimulation of the affected hemisphere. In patients with chronic stroke, we investigated the use of cathodal stimulation of the unaffected hemisphere (UH) to decrease interhemispheric inhibition to the affected hemisphere (AH) and improve motor function and compared these effects with those of anodal stimulation of the affected hemisphere and sham stimulation. Six stroke patients participated in this study.

Using Jebsen Taylor Hand Function Test, motor performance was evaluated before and after active tDCS (20 minutes, 1 mA) applied to the motor cortex of UH (cathodal stimulation) and motor cortex of AH (anodal stimulation) and sham tDCS. Patients showed a significant improvement in their motor performance with their affected hand after anodal tDCS of AH (mean improvement of 9.9%, 95% C.I., 7.13%, 12.8%), and cathodal tDCS of UH (mean improvement of 11.7%, 95% C.I., 5.8%, 17.5%) as compared to sham tDCS. Although cathodal stimulation of UH resulted in an absolute larger improvement in the motor function compared to anodal stimulation of the AH, this difference did not reach significance. There were no side effects. This study supports the notion that tDCS in chronic stroke patients is safe and useful to enhance functional motor recovery by suppressing motor cortical activity in the unaffected hemisphere or stimulating it in the affected hemisphere.

Despite the decline in mortality from stroke, it remains a major challenge for clinicians due to the limited therapeutic options. However, as we showed in the earlier chapters brain stimulation can be a useful therapy in stroke recovery. Additionally, two case-reports have demonstrated positive effects of high frequency epidural stimulation of motor cortex in a stroke patient and in an animal model of acute stroke. Furthermore, a randomized multicenter study of the effects of cortical stimulation on motor function has shown that patients treated with cortical stimulation and rehabilitation therapy had greater improvement in arm function than controls receiving rehabilitation therapy alone. Although cortical electrical stimulation might be a promising therapy for stroke recovery, implantation of an epidural stimulator in stroke patients is expensive and associated with surgical risks. However, non-invasive brain stimulation techniques, such as transcranial magnetic stimulation (TMS) and transcranial direct current stimulation (tDCS), might provide similar benefits without the associated risks of the surgical technique.

A recent study demonstrated that anodal tDCS of the affected hemisphere (AH) can improve motor function of the paretic hand in patients with chronic stroke. In tDCS, the cerebral cortex is stimulated through a weak constant electric current in a non-invasive and painless manner. This weak current can induce changes of cortical excitability—increase or decrease depending on the electrode polarity—that lasts beyond the period of stimulation. Several studies have shown that this technique might modulate cortical excitability in the human motor cortex and visual cortex.

We conjectured that this technique can be used not only to modulate the activity of the damaged hemisphere as previously demonstrated, but also to modulate the activity of the undamaged hemisphere in order to improve motor function. Indeed as we showed earlier, repetitive TMS of the unaffected hemisphere improves motor function in patients with stroke. Additionally, constraint-induced movement therapy (CI therapy) accelerates motor recovery by decreasing the activity of the undamaged hemisphere. This approach of supressing the excitability of the undamaged hemisphere to improve motor recovery in stroke is based on the notion that, after stroke, the nonlesioned hemisphere is disinhibited, perhaps due to the reduction in the transcalosal inhibition from the stroke-damaged hemisphere, and therefore, the UH might increase inhibition to the AH impairing functional recovery. Thus, suppression of the activity of this hemisphere might be beneficial to motor rehabilitation. Because cathodal tDCS can decrease cortical excitability, we hypothesized that cathodal tDCS might decrease the activity of the undamaged hemisphere and therefore decrease the transcalosal inhibition from the damaged to the undamaged hemisphere and therefore improve motor activity and function of the lesioned motor cortex.

Therefore, the aim of the present investigation was (i) to study the effects of cathodal tDCS of the unaffected hemisphere on the motor function in patients with stroke and (ii) to compare it with anodal tDCS of the affected hemisphere, an approach that was reported to improve motor function, and sham tDCS. We report the results of a cross-over, sham stimulation-controlled, double-blinded study assessing the effects tDCS on affected and unaffected hemisphere in patients with chronic stroke.

We studied 6 stroke patients (2 men and 4 women) with mean age of 53.7±16.6 years (±SD) with chronic stroke. The mean time between the stroke and tCDS application was 27.1 months (range of 12 to 72 months). The diagnosis was made by clinical features and confirmed by neuroimaging studies. Classification of the subtypes of strokes was based on the TOAST criteria: four had small vessel occlusion and two had cardioembolic strokes. All subjects were right handed, 3 had right hemispheric and 3 left hemispheric strokes. None of the patients had a history or evidence of dementia or psychiatric disorders. The study was performed in accordance with the declaration of Helsinki (1964). Written informed consent was obtained from all participants prior to inclusion in the study, which was approved by the local ethics committee.

This experimental design was based on a previous study that investigated the effects of anodal tDCS of the affected hemisphere in six patients with stroke. Each patient underwent three different treatments: sham tDCS, anodal tDCS of the affected hemisphere (AH) and cathodal stimulation of the unaffected hemisphere (UH). The order of these conditions was counterbalanced and randomized across subjects. There was an interval of at least 48-hour between each session of tDCS to minimize carryover effects and contamination of the sham stimulation session by a preceding real tDCS session.

Initially, in order to familiarize patients with the Jebsen-Taylor Hand Function Test, they performed this test 10 times. This number of practice sessions was described to be sufficient to reach stable motor performance in patients with stroke. Patients were then randomized to the double-blind, sham controlled part of the experiment. For each condition of stimulation, patients performed the task 3 times for the baseline evaluation, one time during the stimulation and two times after the stimulation. Furthermore, we tested the patients' attention and fatigue using a visual analogue scale (1 to 7, 1=no attention and 7=highest attention and 1=no fatigue and 7=highest level of fatigue). These evaluations were done at baseline, immediately after stimulation (post-1) and after the last Jebsen-Taylor Test (post-2). At the end of the study, patients were asked to guess the order of the condition that they received to detect if they were adequately blinded during this experiment.

Direct current was transferred by a saline-soaked pair of surface sponge electrodes (35 cm2) and delivered by a specially developed, battery-driven, constant current stimulator (Schneider Electronic, Gleichen, Germany) with a maximum output of 10 mA. To stimulate the primary motor cortex (M1), one electrode was placed over the optimal position for induction of motor evoked potentials in the contralateral first dorsal interosseous determined by using transcranial magnetic stimulation. The other electrode was placed was placed over the contralateral orbit. For anodal stimulation, the anode electrode was placed over M1 of the affected hemisphere and cathode electrode was placed over contralateral orbit, whereas for cathodal stimulation, the electrodes were reversed: the cathode was placed over M1 of the unaffected hemisphere and the anode over the contralateral orbit.

These two electrode montages allowed us to test the effects of (i) an increase in cortical excitability in the affected hemisphere (anodal stimulation of AH) and (ii) an decrease in cortical excitability in the unaffected hemisphere (by anodal stimulation of UH) on the motor function. A constant current of 1 mA intensity was applied for 20 min. Subjects felt the current as an itching sensation at both electrodes in the beginning of the stimulation. For the sham stimulation, the electrodes were placed in the same position, however, the stimulator was turned off after 5 seconds as previously described. Therefore the subjects felt the initial itching sensation in the beginning, but received no current for the rest of the stimulation period. This procedure allowed to blind subjects for the respective stimulation condition.

The Jebsen Taylor (JB) Hand Function Test was designed as a broad measure of hand function and is widely used by physical and occupational therapists in clinical practice and clinical trials. This test measures the time taken to perform seven hand tasks, including: 1) writing a sentence, 2) turning over cards, 3) picking up small objects (e.g., pennies, paper clips) and placing them in a container, 4) stacking checkers, 5) simulating eating, 6) moving large empty cans, and 7) moving large weighted cans. Because some of the subjects were using their (pre-stroke) non-dominant hand, the handwriting was excluded as suggested by Kimberley et al (2003) [309] and Hummel et al (2005). The tests were timed with a stop-watch. The patients were instructed to perform as fast as possible with their affected hand while maintaining accuracy. A blinded neuropsychologist—instructed not to communicate with the patient during the task—evaluated patients' performance.

The primary outcome for analysis was change in time of JT test performance. Analyses were done with SAS statistical software (version 8.0, Cary, North Caroline, USA). The distribution of these data were assessed using Wilk-Shapiro test, as this test showed that these data were normally distributed, tests with the assumptions of normal distribution were used. Considering that each patient was evaluated following three different interventions (sham, anodal stimulation of AH, and cathodal stimulation of UH), we used analysis of variance (ANOVA) to test whether there was an overall effect of rTMS type on the primary outcome.

Initially, the difference in the performance was calculated for each patient (difference between post- and pre-stimulation). For the pre-stimulation performance, we averaged the three baseline tests; and for the post-stimulation performance, we averaged the test performed during treatment and the 2 tests performed post-treatment. When appropriate, post-hoc comparisons were carried out using Fisher LSD correction for multiple comparisons.

In a secondary, exploratory analysis (therefore, without P correction), we tested if there was a difference in the task performance across the three post-stimulation evaluations for the three types of stimulation, i.e., a difference across the evaluations performed during and after the stimulation. In order to test for carryover effects, the order effect was assessed by a one-way ANOVA in which the order of stimulation (first, second, and third evaluation collapsed by the type of intervention) was tested. Finally, we tested if there was a correlation between motor function improvement and baseline characteristics, such as time of poststroke and degree of motor deficits using a Pearson correlation coefficient. Data are reported as mean and standard deviation. Statistical significance refers to a two-tailed p value <0.05.

Table 15 summarizes patients' demographics and stroke characteristics. All patients were right handed and all had their strokes after, at least, 12 months. All patients tolerated tDCS without problems and there were no adverse effects related to application of this therapy. F=female; M=male; MMSE=mini-mental state examination; EDS=Edinburgh Handedness Scale; MS=motor strength; ASS=Ashworth Spasticity Score.

TABLE 15

| | | | Time | | | | | |
|---|---|---|---|---|---|---|---|---|
| | Age | | poststroke | | Handedness | | | |
| | (years) | Gender | (months) | MMSE | (EDS) | MS | ASS | Stroke location |
| Patient 1 | 58 | M | 14 | 28 | Right | 4.1 | 1 | Left |
| | | | | | | | | subcortical |
| | | | | | | | | frontal lobe |
| Patient 2 | 75 | M | 39 | 23 | Right | 4.5 | 0 | Left internal |
| | | | | | | | | capsule |
| Patient 3 | 66 | M | 72 | * | Right | 4.3 | 1 | Left frontal |
| | | | | | | | | operculum, |
| | | | | | | | | corona radiata |
| | | | | | | | | and insula |
| Patient 4 | 51 | F | 12.5 | 25 | Right | 4.2 | 1 | Right frontal |
| | | | | | | | | (motor cortex) |
| Patient 5 | 44 | M | 13 | 30 | Right | 4.5 | 0 | Right internal |
| | | | | | | | | capsule |
| Patient 6 | 28 | F | 12 | 24 | Right | 3.5 | 2 | Right corona |
| | | | | | | | | radiata and |
| | | | | | | | | insula |
| Mean | 53.67 | | 27.08 | 26 | | 4.18 | 0.83 | |
| SD | 16.64 | | 24.37 | 2.91 | | 0.37 | 0.75 | |

In order to test whether the motor performance change (difference between post- and pre-treatment) across the different treatments (anodal, cathodal and sham tDCS) were significantly different we performed a one-way ANOVA. This analysis showed that there was a significant main effect (type of stimulation) on JT Test performance (F=10.4, DF=2.10 p=0.0035). Post-hoc comparisons demonstrated that, compared to sham tDCS, there was a significant decrease in performance time after anodal stimulation of AH (p=0.004) and cathodal stimulation of UH (p=0.002). Although cathodal stimulation of the UH hemisphere had a larger absolute improvement (mean, 11.7%, 95% C.I., 5.8%, 17.5%) compared to anodal stimulation of AH (mean, 9.9%, 95% C.I., 7.13%, 12.8%), this difference did not reach statistical significance (p=0.65). After sham stimulation, the mean change in the performance time (from baseline) was not significant −3.6% (95% C.I., −7.8, 0.5).

TABLE 16

| | Anodal tDCS | | | Cathodal tDCS | | | Sham tDCS | | |
|---|---|---|---|---|---|---|---|---|---|
| | | Post- | Change | | Post- | Change | | Post- | Change |
| | Baseline | tDCS | (%) | Baseline | tDCS | (%) | Baseline | tDCS | (%) |
| Patient 1 | 83.33 | 77.65 | 6.81 | 82.56 | 77.06 | 6.66 | 81.15 | 85.42 | −5.27 |
| Patient 2 | 52.68 | 49.75 | 5.55 | 53.10 | 50.47 | 4.95 | 51.54 | 48.35 | 6.19 |
| Patient 3 | 88.99 | 81.13 | 8.83 | 86.91 | 71.86 | 17.31 | 88.99 | 93.98 | −5.60 |
| Patient 4 | 53.99 | 55.99 | −3.70 | 52.62 | 41.95 | 20.27 | 53.99 | 55.55 | −2.89 |
| Patient 5 | 43.51 | 39.27 | 9.75 | 44.67 | 41.91 | 6.19 | 43.51 | 45.84 | −5.37 |
| Patient 6 | 60.33 | 52.17 | 13.54 | 61.91 | 52.77 | 14.77 | 59.20 | 64.26 | −8.55 |
| Mean | 63.80 | 59.33 | 6.80 | 63.63 | 56.00 | 11.69 | 63.06 | 65.57 | −3.58 |
| SD | 18.22 | 16.54 | 5.83 | 17.29 | 15.05 | 6.57 | 17.95 | 19.94 | 5.11 |

Table 16 summarizes Jebsen Taylor Hand Function Test total time results in seconds. Negative change (%) indicates motor performance worsening. In an exploratory analysis, we tested the effect of time of evaluation on the performance task in order to check if the motor performance change would be different during the stimulation compared to the post-stimulation period. A 2-way mixed ANOVA (factor time: baseline, during stimulation, post-1 and post-2, and factor treatment: sham, anodal and cathodal tDCS) with repeated measures on time disclosed a significant interaction time treatment effect (F=3.20, DF=6.45 p=0.011), suggesting a differential effect of treatment on time of evaluation. Indeed, an analysis of each time point showed that the effects of anodal stimulation were more pronounced after, but not during, the stimulation as there was no significant difference between anodal and sham tDCS motor performance in the evaluation during stimulation (p=0.93), but there was a significant difference between these two treatments in the evaluation post-1 (p=0.002) and post-2 (p=0.001). Whereas, for cathodal stimulation, the motor performance improvement compared to sham stimulation was significantly different from the evaluation during stimulation (p=0.02) to the evaluation post-1 (0.02) and post-2 (0.02) (FIG. 2).

Finally, we tested if our design was associated with an order effect. The motor performance was evaluated considering the order of stimulation (first, second, and third condition). The result of the one-way ANOVA showed that there was no main effect of order of stimulation (first, second, and third) on motor performance (F=0.07, DF=2.15, p=0.93).

To test if the repeated testing of the patients could be associated with a decrease in the attention or increase in the fatigue that could have confounded our results, we asked the patients to score their attention and fatigue in a visual analogue scale before the test (baseline), immediately after stimulation (post-1), and after the last test (post-2). The results showed that patients consistently kept a good attention across repeated testing and different treatments (mean and SD, 5.56±1.29—7 is the maximal attention) and did not complain of fatigue (mean and SD, 2.09±1.36—1 indicates no fatigue and 7 maximal fatigue). A two-way ANOVA (factor stimulation—sham, anodal, cathodal tDCS, and factor time—first, second, and third evaluation) showed that there was no main effect of stimulation nor time on attention (F=1.04, DF=2.14, p=0.38 for time effect, and F=0.05, DF=2.15, p=0.95 for stimulation effect) and fatigue (F=1.62, DF=2.14, p=0.23 for time effect, and F=0.12, DF=2.15, p=0.88 for condition effect), suggesting that attention and fatigue were not confounders of results in this study.

We tested whether there was a correlation between the motor improvement after anodal stimulation of AH and cathodal stimulation of UH with poststroke duration and the degree of motor deficit. The results showed that there was not a correlation between anodal stimulation of AH and either the poststroke duration (r=0.02, p=0.95) or the degree of motor deficit (r=0.34, p=0.49). Additionally, there was no correlation between cathodal stimulation of UH and either the poststroke duration (r=0.25, p=0.62) or the degree of the motor deficit (r=0.52, p=0.28). In addition, we assessed whether the improvement of the tasks from the JT tests that demanded more proximal function, such as turning cards, picking up pennies, and stacking checkers could be correlated to the degree of motor deficit at the baseline. These analyses showed that there was not a correlation between the motor performance improvement in the proximal tasks and the degree of motor deficit after either anodal stimulation (r=0.21, p=0.74) or cathodal stimulation (r=0.66, p=0.22). Although we might have been underpowered to perform these tests, the results are not even close to statistical significance. These findings suggest that motor function improvement induced by tDCS was independent from stroke's time course or influence on motor performance.

This study on patients with well established deficits at least 12 months after a stroke demonstrates that cathodal tDCS of the unaffected motor cortex and anodal stimulation of the affected motor cortex can significantly improve hand motor function as compared with sham stimulation. Importantly, there were no adverse effects and tDCS of either the affected or unaffected hemisphere was well tolerated by all the stroke patients.

This study replicates and extends the findings of the study by [283]. In this study, stroke patients underwent anodal tDCS of the affected hemisphere and there was a significant improvement of the motor function compared to sham tDCS as indexed by Jebsen Taylor Hand Function Test. Our study investigated a different population of stroke patient than the study of Hummel et al., as our patients were more severely impaired (the mean motor score was 4.2 in our study vs. 4.8 in Hummel's study) and shorter duration of stroke, on average (mean time after stroke was 27.1 months in our study vs. 44.3 in Hummel's study). Therefore, the similar results between these two studies speaks against that these findings are due by chance. Furthermore, we expanded the findings of Hummel et al. as we demonstrated that cathodal stimulation of the contralateral hemisphere can yield similar motor improvement as compared to anodal stimulation of the AH.

This finding is important as the anatomy changes following stroke in the affected hemisphere could possibly disturb the electric fields generated by tDCS and therefore the results from this stimulation might be less predictable. Indeed it was recently demonstrated that electric current induced by TMS is modified in magnitude, location, and orientation in the lesioned hemisphere [164]. Although TMS and tDCS are two vastly different stimulation techniques with completely different sources of stimulation, the modifications to the conductive matrix of the tissues that altered the TMS induced currents will have an analogous altering effect on the tDCS generated electric fields. Given this electric field alteration in the areas near stroke, one could speculate that cathodal stimulation of the UN could be more predictable than anodal stimulation of the AH. Indeed, although cathodal stimulation of the UH had a larger effect, this was not significant; however, a sample size consideration and, thus error type II, should be accounted when analyzing the lack of the difference between these two conditions.

It is interesting to note the differential effects of anodal stimulation of AH and cathodal stimulation of UH on time of motor improvement. Whereas anodal stimulation of AH had the most pronounced motor effect after stimulation, cathodal stimulation of UH had an apposite effect: more pronounced effect during the stimulation compared to after stimulation. Some reasons might explain this finding. First, as we showed in the previous chapter, anatomical changes in the affected hemisphere could perturb the electric current and delay the tDCS effects on the neural elements of the damaged hemisphere. Second, this divergence might be explained by fundamental differences in the mechanisms of action of cathodal and anodal stimulation—for instance, Liebetanz et al. (2002) showed that carbamazepine blocks the effects of anodal stimulation selectively, therefore, suggesting that anodal, not cathodal, stimulation induces a depolarization of membrane potentials. Finally, this finding should be interpreted in a context of an exploratory analysis in which the P value was not corrected, therefore, this result has to be replicated by further studies.

The use of cathodal stimulation on the unaffected hemisphere was based on the hypothesis that this stimulation would suppress activity locally and release the damaged hemisphere from possible excessive transcallosal inhibition, causing some functional improvement. This notion is in agreement with several recent neuroimaging studies that show that strong activation of the ipsilateral motor cortex after stroke during motor tasks is associated with poor motor recovery; rTMS study in normal subjects that showed a shortening of execution time of an overlearned motor task after slow rTMS of the ipsilateral motor cortex; and an rTMS study in which stroke patients showed an improvement of the motor function after low-frequency rTMS of the unaffected hemisphere. Furthermore, converging evidence that the modulation of the healthy hemisphere can be beneficial to motor function recovery comes from Constraint-Induced therapy (CI therapy). This therapy has been shown to be efficacious in stroke patients. In CI therapy, motor improvement is thought to result from the forced use of the paretic limb and the underuse of the non-paretic limb, and is associated with a modulation of inter-hemispheric excitability. Immobilization of a body part may result in a reduction of the excitability of the contralateral motor cortex due to the decreased efferent demand and afferent input.

Brain stimulation techniques might be a promising intervention in stroke recovery. Transcranial direct current stimulation has an advantage as this technique is inexpensive, easy to administer and painless. In the next and concluding chapter we compare and contrast the different forms of brain stimulation as treatment for neuropathologies.

Applicant presented an analysis of a realistic head model based on an MRI derived geometry integrated with variable tissue electric properties. The model provided evidence for the existence of currents normal to the cortical interface, demonstrated the effect of the tissue boundaries on the induced current, and allowed one to test the predictions of alpha dispersion theory. This model showed the importance of ascertaining the true in-vivo tissue values and showed the importance of the surrounding tissues on the final current density during stimulation.

Many of the results found in the study were contrary to common theory in the TMS world and thus the clinical impacts of the electromagnetic effects were reviewed. For instance, clinically it was believed that radial currents are minimized during stimulation and as such misconceptions precipitated about I and D wave theories of stimulation. However our results show that I and D wave differences are not caused by the absence of radial currents during stimulation.

Applicant also presented a preliminary experiment focused on ascertaining the dispersive properties of head tissues in-vivo. A study was presented where impedance measurements were made of the brain tissue, skull, and skin in a living animal during surgery. The values that were recorded differed by 1-2 orders of magnitude from the Brooks Air Force Database. More importantly, the values were shown to drastically change following an ischemic event by approximately an order of magnitude over a few hours of time. While the values that we recorded suggest the possibility of displacement currents during TMS we based our primary analysis on tissue values which excluded their possibility.

Applicant explored the effect that electrical and anatomical changes caused by stroke have on the TMS induced electrical currents in the brain. Our models were based on a finite element electromagnetic solver integrated with MRI derived head models. We focused on the difference between the healthy head model and the stroke models. We showed that the disruption due to a stroke can drastically modify the effect of TMS in several ways: (1) it alters the location of the maximum cortical current density, (2) it alters the magnitude and distribution of the induced currents, and (3) it modifies the focus of stimulation, all of which will alter the population of neural elements stimulated and ultimately lead to clinical implications. We also showed that stimulation contralateral to the infarction was unaffected and that served for the basis of the next chapter.

Applicant presented a study where we investigated the use of 1 Hz rTMS to the unaffected hemisphere to decrease interhemispheric inhibition of the lesioned hemisphere and improve motor function in patients within 12 months after a stroke. The patients showed a true benefit in motor performance following stimulation and rTMS could thus make a potentially powerful tool for neurorehabilitation in the future. We are currently completing further studies in this area.

Applicant explored the effect that electrical and anatomical changes caused by atrophy have on the TMS induced electrical currents in the brain. Our models were based on a finite element electromagnetic solver integrated with MRI derived head models. The results demonstrate that TMS induced currents in the cortex can be modified in magnitude, location, and orientation in situations of brain atrophy. These cortical current density perturbations could prove to be dangerous or at the very least lead to unreliable results if guided by conventional methods based on healthy head models or with simplified models of atrophy which ignore electromagnetic field-tissue interactions.

Applicant explored the behavior of the tDCS cortical current densities. Our models were based on a finite element electromagnetic solver integrated with MRI derived head models (including three different stroke cases and an atrophy case). The models explored numerous aspects of the current densities including the effects of varied electrode schemes used before in human studies and also hypothetical montages that might be used in future studies, the effects of varied electrode sizes, the shunting effect in the tissues, and the effects of stroke on the stimulating current density distributions. Overall the technique was found to show potentially promising uses, one of which we explored in the following chapter.

Applicant also investigated the use of cathodal stimulation of the unaffected hemisphere (UH) to decrease interhemispheric inhibition to the affected hemisphere (AH) and improve motor function and compared these effects with those of anodal stimulation of the affected hemisphere and sham stimulation in patients with chronic stroke. Patients showed a significant improvement in their motor performance with their affected hand after anodal tDCS of AH (mean improvement of 9.9%), and cathodal tDCS of UH (mean improvement of 11.7%) as compared to sham tDCS. Quite conceivably, the difference in improvements seen when the different hemispheres were treated could have been caused by the disruption in current densities seen in the infarcted hemisphere as examined in the previous chapter. The patients showed a true benefit in motor performance following stimulation and tDCS could also make a potentially powerful tool for neurorehabilitation in the future.

As discussed above, the studies described in the foregoing present findings related to various embodiments according to the present invention. Requirements recited in the above examples and applications of the various aspects of the invention pertain only to the particular embodiment being described and do not express limitations on the invention as a whole. The data, features, components and implementations in the above studies are mentioned merely to provide examples of the various aspects of the present invention. However, the aspects of the invention are not limited to the reports in the above studies.

It should be appreciated from the foregoing, there are numerous aspects of the present invention described herein that can be used independently of one another or in any combination. In particular, any of the above described operations or components may be employed in any of numerous combinations and procedures. For example, according to one embodiment, a portable headpiece or helmet unit, customized for an individual, is adapted for self-delivery of TMS treatment by the patient. The helmet is custom fit to the patient's head and encapsulates a TMS coil positioned to target a desired brain region with precision and consistency. The inner portion of the helmet may be tailored to an individual's anatomy without having to adjust an outer portion of the helmet (which may be reused). The helmet includes communications means to facilitate remote interactivity and control.

The TMS coil is positioned using an electromagnetic field model that incorporates dielectric properties of the subject's brain and/or conductivity anisotropy to facilitate positioning the TMS coil to stimulate the desired portion of the brain. The helmet also includes built-in electrodes for obtaining EEG measurements that allow a clinician to monitor the EEG and control the stimulation process based on the EEG, and remotely adjust the protocol based on patient response. However, it should be appreciated that other embodiments may not include one or more of the above components, or utilize one or more of the above operations. A headpiece may include any one or combination of the above features, as the aspects of the invention are not limited in this respect. It should also be appreciated that in some embodiments, all of the above-described components, features and operations can be used together in any sequence, or any combination or subset of the components, features and operations described above can be employed together in a particular implementation, as the aspects of the present invention are not limited in this respect.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

It should be appreciated that the various methods outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code. In this respect, it should be appreciated that one embodiment of the invention is directed to a computer-readable medium or multiple computer-readable media (e.g., a computer memory, one or more floppy disks, compact disks, optical disks, magnetic tapes, etc.) encoded with one or more programs that, when executed, on one or more computers or other processors, perform methods that implement the various embodiments of the invention discussed above. The computer-readable medium or media can be transportable, such that the program or programs stored thereon can be loaded onto one or more different computers or other processors to implement various aspects of the present invention as discussed above.

It should be understood that the term “program” is used herein in a generic sense to refer to any type of computer code or set of instructions that can be employed to program a computer or other processor to implement various aspects of the present invention as discussed above. Additionally, it should be appreciated that according to one aspect of this embodiment, one or more computer programs that, when executed, perform methods of the present invention need not reside on a single computer or processor, but may be distributed in a modular fashion amongst a number of different computers or processors to implement various aspects of the present invention.

Various aspects of the present invention may be used alone, in combination, or in a variety of arrangements not specifically discussed in the embodiments described in the foregoing, and the aspects of the present invention described herein are not limited in their application to the details and arrangements of components set forth in the foregoing description or illustrated in the drawings. The aspects of the invention are capable of other embodiments and of being practiced or of being carried out in various ways.

The phraseology and terminology used herein is for the purpose of description and should not be regarded as limiting. The use of “including,” “comprising,” or “having,” “containing,” “involving,” and variations thereof herein, is meant to encompass the items listed thereafter and equivalent thereof as well as additional items.

What is claimed is:

1. A portable transcranial magnetic stimulation (TMS) device for delivering a TMS procedure, the portable TMS device comprising:
at least one coil that, when energized, generates electromagnetic energy;
a helmet adapted to fit a user's head and configured to hold the at least one coil in a predetermined position with respect to the user's head; and
a port having at least one power connection coupled to the at least one coil, the at least one power connection adapted to connect the at least one coil to a power source capable of energizing the at least one coil, the port further comprising at least one data connection adapted to exchange data with at least one external component, the port being located on the helmet.

2. The portable TMS device of claim 1, wherein the helmet comprises an inner portion customizable to the user's head, and an outer portion adapted to hold the at least one coil in the predetermined position.

3. The portable TMS device of claim 2, wherein the outer portion includes moldable plastic that conforms to the at least one coil.

4. The portable TMS device of claim 2, wherein the outer portion includes a hollow region to contain the at least one coil, the hollow region capable of being filled by a foam to hold the at least one coil in the predetermined position.

5. The portable TMS device of claim 2, wherein the outer portion includes a hollow region to contain the at least one coil, the hollow region capable of being removed of air to form a vacuum to hold the at least one coil in the predetermined position.

6. The portable TMS device of claim 1, further comprising a controller configured to control the application of power from the power source to energize the at least one coil according to a desired TMS procedure.

7. The portable TMS device of claim 6, wherein the controller is located on the helmet.

8. The portable TMS device of claim 7, wherein the controller includes at least one processor configured to execute at least one program that, when executed, provides instructions to the controller that operates the at least one coil according to the desired TMS treatment.

9. The portable TMS device of claim 8, further comprising at least one treatment program that, when executed, performs a respective TMS procedure.

10. The portable TMS device of claim 9, wherein the treatment program is stored on the external component and information to perform the respective TMS procedure is exchanged between the external component and the controller via the data connection.

11. The portable TMS device of claim 9, wherein the controller includes a memory to store the at least one treatment program.

12. The portable TMS device of claim 11, wherein the data connection includes a network connection and the at least one treatment program is transferred to the memory over a network from at least one network device.

13. The portable TMS device of claim 6, further comprising at least one electrode positioned within the helmet so as to make contact with the user's head, the at least one electrode capable of generating EEG signals of a portion of the user's brain.

14. The portable TMS device of claim 13, wherein the controller is configured to receive the EEG signals and to control the operation of the at least one coil based, at least in part, on the EEG signals.

15. A method of positioning a coil with respect to a person's head to target a desired region of the person's brain with transcranial magnetic stimulation (TMS), the method comprising:
obtaining a dielectric property map of a portion of the brain, the dielectric map indicating a spatial distribution of at least one dielectric property over the portion of the brain;
determining a location for the coil based, at least in part, on the dielectric property map, the location being such that when the coil is positioned at the location, magnetic energy generated by the coil is focused on the desired region; and
positioning the coil at the determined location.

16. The method of claim 15, wherein the at least one dielectric property includes at least one of conductivity, permittivity and permeability.

17. (canceled)

18. The method of claim 16, wherein obtaining the dielectric property map includes obtaining the dielectric property map, at least in part, from information in a magnetic resonance image (MRI) of the portion of the brain.

19. The method of claim 16, wherein obtaining the dielectric property map includes obtaining the dielectric property map, at least in part, from performing electrical measurements on the portion of the brain.

20. The method of claim 19, wherein performing electrical measurements includes applying electrical signals to the brain and performing at least one impedance measurement resulting from the applied electrical signals.

21. The method of claim 16, further comprising obtaining at least one conductivity anisotropy measurement, and wherein determining the location includes determining the location based, at least in part, on the at least one conductivity anisotropy measurement.

22. (canceled)
